SEQUENCE LISTING (16 sequences)

SEQ ID NO: 1 - DNA, 21 bp, Artificial Sequence
  forward primer
aggagcgatttgctggtgtgg

SEQ ID NO: 2 - DNA, 20 bp, Artificial Sequence
  reverse primer
ctaccagggcctttgagatg

SEQ ID NO: 3 - DNA, 20 bp, Artificial Sequence
  forward primer
aacagctcatgaggctacgg

SEQ ID NO: 4 - DNA, 20 bp, Artificial Sequence
  reverse primer
tgggtcttgaggacctctgt

SEQ ID NO: 5 - DNA, 21 bp, Artificial Sequence
  forward primer
ctactcccaggttctcttcaa

SEQ ID NO: 6 - DNA, 21 bp, Artificial Sequence
  reverse primer
gcagagaggaggttgactttc

SEQ ID NO: 7 - DNA, 20 bp, Artificial Sequence
  forward primer
cctgctgcactttggagtga

SEQ ID NO: 8 - DNA, 21 bp, Artificial Sequence
  reverse primer
gagggtttgctacaacatggg

SEQ ID NO: 9 - DNA, 22 bp, Artificial Sequence
  forward primer
taggctggagagctacaagagg

SEQ ID NO: 10 - DNA, 20 bp, Artificial Sequence
  reverse primer
agtgcttgaggtggttgtgg

SEQ ID NO: 11 - DNA, 23 bp, Artificial Sequence
  forward primer
agaatcaccagcagcaagtgtcc

SEQ ID NO: 12 - DNA, 24 bp, Artificial Sequence
  reverse primer
atggaatcctgaacccacttctgc

SEQ ID NO: 13 - DNA, 21 bp, Artificial Sequence
  forward primer
gatggatgctaccaaactgga

SEQ ID NO: 14 - DNA, 20 bp, Artificial Sequence
  reverse primer
ggaaattggggtaggaagga

SEQ ID NO: 15 - DNA, 20 bp, Artificial Sequence
  forward primer
gacagccactcacctcttca

SEQ ID NO: 16 - DNA, 20 bp, Artificial Sequence
  reverse primer
cctctttgctgctttcacac

(19) United States
(12) Patent Application Publication
(10) Pub. No.: US 2024/0325389 A1
(43) Pub. Date: Oct. 3, 2024

(54) NEW THERAPEUTIC TARGETS WITH AN ANTI-INFLAMMATORY AND ANTI-INTERFERON EFFECT

(71) Applicants: UNIVERSITÉ PARIS CITÉ, PARIS (FR); CENTRE NATIONAL DE LA RECHERCHE SCIENTIFIQUE, PARIS (FR); INSTITUT NATIONAL DE LA SANTE ET DE LA RECHERCHE MEDICALE, PARIS (FR)

(72) Inventors: Jean-Philippe HERBEUVAL, PARIS (FR); Benoit SCHNEIDER, ANTONY (FR); Nassima BEKKADOUR, MARMANDE (FR); Vincent BAUDOUIN, LE KREMLIN BICETRE (FR)

(21) Appl. No.: 17/907,324

(22) PCT Filed: Mar. 26, 2021
(86) PCT No.: PCT/EP2021/058039

§ 371 Date: Oct. 3, 2022

(30) Foreign Application Priority Data
Mar. 27, 2020 (FR) FR2003087

(51) Int. Cl. A61K 31/506; A61K 31/404; A61K 31/4409; A61K 31/4439; A61K 45/06; A61P 19/02; A61P 19/06; A61P 29/00; A61P 31/18; A61P 37/06

(52) U.S. Cl. CPC A61K 31/506; A61K 31/404; A61K 31/4409; A61K 31/4439; A61K 45/06; A61P 19/02; A61P 19/06; A61P 29/00; A61P 31/18; A61P 37/06

(57) ABSTRACT

The present invention relates to an inhibitor of the ROCK-PDK1 protein kinase complex for use thereof as an anti-inflammatory and anti-interferon agent. The present invention also relates to a pharmaceutical composition comprising an inhibitor of the ROCK-PDK1 protein kinase complex as active principle and at least one pharmaceutically acceptable excipient and/or carrier and/or a diluent and/or a pharmaceutically acceptable vehicle. The present invention also relates to the use of said one pharmaceutical composition in the prevention and/or the treatment of inflammatory diseases, viral and/or bacterial infections and autoimmune diseases.

### REFERENCE TO SEQUENCE LISTING SUBMITTED ELECTRONICALLY

The instant application contains a Sequence Listing which has been submitted electronically in text format and is hereby incorporated by reference in its entirety. Said text copy, created on February 14, is named 17907324_SL.txt and is 3341 bytes in size.

### TECHNICAL FIELD

The present invention relates to an inhibitor of the ROCK-PDK1 protein kinase complex and use thereof as an anti-inflammatory and anti-interferon agent. The present invention also relates to a pharmaceutical composition comprising an inhibitor of the ROCK-PDK1 protein kinase complex and use thereof in the prevention and/or the treatment of inflammatory diseases, viral and/or bacterial infections and autoimmune diseases.

### PRIOR ART

Inflammation is a beneficial physiological process for the organism to the extent that it is transient. It makes it possible for example to combat bacterial or viral infections. However, many pathologies affecting several organs such as the intestine, the central nervous system, the osteoarticular system, the skin, the lungs, etc. are associated with chronic inflammation of the tissues. Chronic inflammation affects the homeostasis of the tissues, leading to functional abnormalities such as digestive disorders, aggravation of neurological or neurodegenerative diseases, a motor disability, skin lesions, respiratory insufficiency, etc. Chronic inflammation of the tissues results from the continuous production of inflammatory cytokines (TNFα, interleukins, interferons) by the immune cells or similar cells (microglial cells in the central nervous system), which leads to deterioration of the cells that are sensitive to the inflammatory factors. Very few medicaments (steroid and non-steroid) currently make it possible to contain the inflammatory response (aspirin, ibuprofen, paracetamol, corticosteroids), some of which give rise to non-negligible side effects in humans.

Consequently, it appears necessary to have available new compositions capable of containing the inflammatory response, in particular by preventing or blocking the synthesis of inflammatory cytokines, so as to prevent and/or treat inflammatory diseases, viral and/or bacterial infections and autoimmune diseases, while reducing the risks of side effects when they are administered to the patient.

Quite surprisingly, the inventors have shown that the inhibitors of the ROCK-PDK1 protein kinase complex are capable of preventing and/or inhibiting and/or blocking the synthesis of the inflammatory cytokines, such as tumour necrosis factor alpha (TNFα), interleukin 1β (IL-1β) and interleukin 6 (IL-6) but also the different sub-types of interferons (IFN), such as interferon alpha (type I), interferon gamma (type II) and interferon lambda (type Ill).

Thus the subject of the present invention is an inhibitor of the ROCK-PDK1 protein kinase complex for use thereof as an anti-inflammatory and anti-interferon agent.

Thus the subject of the present invention is an inhibitor of the ROCK-PDK1 protein kinase complex for use thereof as an anti-inflammatory and anti-interferon agent, characterized in that said inhibitor of the ROCK-PDK1 protein kinase complex is an inhibitor of the PDK1 protein kinase, and in that the inhibitor of the PDK1 protein kinase inhibits the production and/or the secretion of inflammatory cytokines and of interferons by an immune cell, when it is placed in contact with said immune cell.

The ROCK and PDK1 kinases are known to have a deleterious role in the prion neurodegenerative diseases and Alzheimer's. Overactivation of the ROCK and PDK1 kinases makes the diseased neurones very vulnerable to the inflammatory cytokine TNFα as a result of TNFα (TNFR) receptors clustering at the cell surface membrane. Pharmacological inhibition of the ROCK protein kinase or the PDK1 protein kinase has the effect of desensitizing the diseased neurones from the toxic effects of TNFα while reducing the amount of TNFR at the plasma membrane of the diseased neurones and thus increasing the survival of the neurones.

Within the meaning of the present invention, by “inhibitor” is meant a compound capable of inhibiting or blocking or neutralizing the expression of the targeted metabolic activity; the metabolic activity being weaker, or even zero in the presence of the inhibitor. Advantageously, the inhibitor of the ROCK-PDK1 protein kinase complex induces a reduction in the expression of the ROCK and PDK1 kinase protein activity while stimulating dissociation of the ROCK-PDK1 complex. Advantageously, the inhibitor of the ROCK-PDK1 protein kinase complex is neutralizing. In particular, it inhibits or neutralizes the biological function of the ROCK-PDK1 protein kinase complex, which has the consequence of protecting the cells from the TNFα inflammatory stress. The neutralization capacity of the inhibitor can be tested by a standard test, in particular by measuring the capacity of the inhibitor to dissociate the ROCK-PDK1 protein kinase complex and to reduce the enzyme activity of the ROCK and PDK1 kinases (IC50). Advantageously, the inhibitor presents an IC50 comprised between 11 nM and 30 nM.

Advantageously, the inventors have shown that the selective inhibition of the interaction between the ROCK and PDK1 protein kinases inhibits the secretion of the inflammatory cytokines (TNFα, interleukin 1β (IL-1β), interleukin 6 (IL6), MCP1 (CCL2)) and of the interferons (IFN) by the activated immune cells, showing a beneficial effect of the ROCK or PDK1 inhibitors according to the invention with respect to the inflammatory stress without impact on the viability of said immune cells. Advantageously, the inhibitor of the ROCK-PDK1 protein kinase complex inhibits the secretion of inflammatory cytokines and of the interferons by an activated immune cell in vitro and ex vivo, in particular when the inhibitor of the ROCK-PDK1 protein kinase complex is placed in contact with the immune cell.

Advantageously, the inventors have shown that placing immune cells in contact with an inhibitor of the ROCK-PDK1 protein kinase complex according to the invention inhibits the secretion of the inflammatory cytokines (TNFα, interleukin 1β (IL-1β), interleukin 6 (IL6), MCP1 (CCL2)) and of the interferons (IFN) by said immune cells, in comparison with immune cells which have not been placed in contact with said inhibitor of the ROCK-PDK1 protein kinase complex according to the invention.

Advantageously, the inventors have shown that placing immune cells in contact with an inhibitor of the PDK1 protein kinase according to the invention inhibits the secretion of the inflammatory cytokines (TNFα, interleukin 1β (IL-1β), interleukin 6 (IL6), MCP1 (CCL2)) and of the interferons (IFN) by said immune cells, in comparison with immune cells which have not been placed in contact with said inhibitor of the PDK1 protein kinase according to the invention.

Advantageously, the inventors have shown that placing immune cells in contact with an inhibitor of the PDK1 protein kinase of formula (IV) (also called BX912) inhibits the secretion of the inflammatory cytokines (TNFα, interleukin 1β (IL-1β), interleukin 6 (IL6), MCP1 (CCL2)) and of the interferons (IFN) by said immune cells, in comparison with immune cells which have not been placed in contact with said inhibitor of the PDK1 protein kinase according to the invention.

In a particularly advantageous embodiment of the invention, the inhibitors of the ROCK-PDK1 protein kinase complex allow selective inhibition of the interaction between the ROCK and PDK1 protein kinases. Advantageously, the inventors have shown that the selective inhibition of the interaction between the ROCK and PDK1 protein kinases also prevents the synthesis of the inflammatory cytokines (TNFα, interleukins 1β and 6 (IL-1β and IL6), MCP1 (CCL2)) and of the interferons, showing a beneficial effect of the ROCK or PDK1 inhibitors according to the invention with respect to the inflammatory stress without toxic impact.

Within the meaning of the present invention, by “anti-inflammatory agent” is meant an agent capable of preventing and/or blocking and/or inhibiting the synthesis of the inflammatory cytokines, and in particular an agent capable of preventing and/or blocking and/or inhibiting the synthesis of the tumour necrosis factor alpha (TNFα), of interleukin 1β (IL-1β), of MCP1 (CCL2) and of interleukin 6 (IL-6).

Advantageously, by “anti-inflammatory agent” is meant an agent capable of preventing and/or blocking and/or inhibiting the secretion of the inflammatory cytokines, and in particular an agent capable of preventing and/or blocking and/or inhibiting the secretion of the tumour necrosis factor alpha (TNFα), of interleukin 1β (IL-1β), of interleukin 6 (IL-6), of MCP1 (CCL2), by an immune cell, when said agent is placed in contact with an immune cell.

Within the meaning of the present invention, by “anti-interferon agent” is meant an agent capable of preventing and/or blocking and/or inhibiting the synthesis of the interferons (IFN), and in particular an agent capable of preventing and/or blocking and/or inhibiting the synthesis of interferon alpha (type I), of interferon gamma (type II) and of interferon lambda (type Ill). Advantageously, by “anti-interferon agent” is meant an agent capable of preventing and/or blocking and/or inhibiting the secretion of the interferons, and in particular an agent capable of preventing and/or blocking and/or inhibiting the secretion of interferon alpha (type I), of interferon gamma (type II) and of interferon lambda (type Ill) by an immune cell, when said agent is placed in contact with an immune cell.

Within the meaning of the present invention, by “anti-inflammatory agent and anti-interferon agent” is meant an agent capable of preventing and/or blocking and/or inhibiting the synthesis of the inflammatory cytokines and interferons, and in particular an agent capable of preventing and/or blocking and/or inhibiting the synthesis of the tumour necrosis factor alpha (TNFα), of interleukin 1β (IL-1β), of interleukin 6 (IL-6), of MCP1 (CCL2), of interferon alpha (type I), of interferon gamma (type II) and of interferon lambda (type Ill). Advantageously, by “anti-inflammatory agent and anti-interferon agent” is meant an agent capable of preventing and/or blocking and/or inhibiting the secretion of the inflammatory cytokines, and in particular an agent capable of preventing and/or blocking and/or inhibiting the secretion of the tumour necrosis factor alpha (TNFα), of interleukin 1β (IL-1β), of interleukin 6 (IL-6), of MCP1 (CCL2), of interferon alpha (type I), of interferon gamma (type II) and of interferon lambda (type Ill) by an immune cell, when said agent is placed in contact with an immune cell.

Within the meaning of the present invention, by “immune cell” or “cell of the immune system” or “immunity cell” is meant any cell of the immune system that originates from a hematopoietic stem cell in the bone marrow.

Advantageously, the immune cells according to the invention are peripheral blood mononuclear cells (PBMCs). The term “peripheral blood mononuclear cells” (PBMCs) refers to peripheral blood cells having a round nucleus. These mononuclear blood cells recirculate between the tissues and the blood and are an essential element of the immune system for combatting infections and adapting to intruders. There are two main types of mononuclear cells: the lymphocytic cells and the myeloid cells.

The lymphocytic population of the PBMCs is generally composed of T cells, B cells and NK cells. The myeloid population of the PBMCs is composed of dendritic cells (myeloid and plasmacytoid) and monocytes/macrophages. The PBMCs can be isolated from total blood samples by methods that are well known in the art (for example, Ficoll density gradient). Advantageously, the immune cell is a T lymphocyte. Advantageously, the immune cell is a B lymphocyte.

Within the meaning of the present invention, the terms “T lymphocyte” and “T cell” are used interchangeably and refer to a main type of white blood cells, which reach maturation in the thymus and which have various roles in the immune system, including the identification of specific foreign antigens in the body and the activation and/or deactivation of other immune cells. The T lymphocyte can be any T cell whatsoever selected from: a cultured T cell, for example, a primary T cell, or a T cell from a cultured T cell line, for example Jurkat, SupTI, etc. or a T cell obtained from a mammal. The T cells can be CD3+ cells. The T cell can be any type of T cell whatsoever and can be at any stage of development whatsoever, including, but without being limited to, CD4+/CD8+ double positive T cells, CD4+ helper T cells (for example Th1 and Th2 cells), CD8+ T cells (for example cytotoxic T cells), a T cell present among the peripheral blood mononuclear cells (PBMCs), the peripheral blood leukocytes (PBL), a T cell forming part of the tumour infiltrating lymphocytes (TIL), the memory T cells, the naive T cells, the regulatory T cells, the gamma delta T cells (Tyδ cells). Additional types of helper T cells comprise cells such as the Th3, Th17, Th9 or Tfh cells. Additional types of memory T cells comprise cells such as the central memory T cells (Tcm cells), effector memory T cells (Tem and TEMRA cells). “T cell” can also denote a genetically modified T cell, such as a T cell modified to express a T cell receptor (TCR) or a chimeric antigen receptor (CAR). The T cell can also be differentiated from a stem cell or from a progenitor cell.

Within the meaning of the present invention, by “B cell” or “B lymphocytic cell” or “B lymphocyte” is meant a lymphocytic cell that is produced in the bone marrow, product of the immunoglobulins and is involved in the production of antibodies during the humoral immune response. The B lymphocyte can be any B cell whatsoever, selected from a stem B cell, a pro-B cell, a pre-B cell, a naive B cell, an activated B cell, a GC memory B cell, a late plasmablastic cell and a plasma cell.

Within the meaning of the present invention, the term “placed in contact”, when it is used with reference to an action carried out on an immune cell, is used interchangeably to denote the culturing, incubation or exposure of an immune cell with one or more of the inhibitors of the ROCK-PDK1 protein kinase complex according to the invention. Advantageously, the term “placed in contact”, when it is used with reference to an action carried out on an immune cell, is used interchangeably to denote the culturing, incubation or exposure of an immune cell with one or more of the inhibitors of the PDK1 protein kinase according to the invention.

Within the meaning of the present invention, a cell “not placed in contact” or “not treated” is a cell that has not been treated, for example, cultured, placed in contact or incubated with one or more of the inhibitors of the ROCK-PDK1 protein kinase complex according to the invention. The cells placed in contact with a stimulation agent, such as R848, CpG-A, or placed in contact with another vehicle are examples of cells placed in contact.

Within the meaning of the present invention, by “synthesis of inflammatory cytokines” or “production of inflammatory cytokines” or “secretion of inflammatory cytokines” is meant the release in the external environment of inflammatory cytokines by a cell, advantageously an immune cell.

Within the meaning of the present invention, by “synthesis of interferon” or “production of interferon” or “secretion of interferon” is meant the release in the external environment of interferon by a cell, advantageously an immune cell.

In an advantageous embodiment of the invention, the inhibitor of the ROCK-PDK1 protein kinase complex is selected from an inhibitor of the ROCK protein kinase, an inhibitor of the PDK1 protein kinase, an agent dissociating the ROCK-PDK1 protein kinase complex and a combination thereof.

Within the meaning of the present invention, by “inhibitor of the ROCK protein kinase” is meant a compound capable of inhibiting or blocking or neutralizing the expression of the metabolic activity of the ROCK protein kinase. Advantageously, the capacity of the inhibitor to neutralize the ROCK protein kinase (IC50) is comprised between 11 nM and 30 nM. Advantageously, the inhibitor of the ROCK protein kinase is a compound capable of inhibiting or blocking or neutralizing the expression of the metabolic activity of the ROCK protein kinase of an immune cell. Advantageously, the capacity of the inhibitor to neutralize the ROCK protein kinase of an immune cell (IC50) is comprised between 11 nM and 30 nM.

Within the meaning of the present invention, by “inhibitor of the PDK1 protein kinase” is meant a compound capable of inhibiting or blocking or neutralizing the expression of the metabolic activity of the PDK1 protein kinase. Advantageously, the capacity of the inhibitor to neutralize the PDK1 protein kinase (IC50) is comprised between 11 nM and 30 nM. Advantageously, the inhibitor of the PDK1 protein kinase is a compound capable of inhibiting or blocking or neutralizing the expression of the metabolic activity of the PDK1 protein kinase of an immune cell. Advantageously, the capacity of the inhibitor to neutralize the PDK1 protein kinase of an immune cell (IC50) is comprised between 11 nM and 30 nM.

Within the meaning of the present invention, by “agent dissociating the ROCK-PDK1 protein kinase complex” is meant an intercalating agent capable of dissociating the ROCK-PDK1 protein kinase complex, fixing itself in the zone of interaction between the ROCK-PDK1 protein kinases, leading to a steric clash and separating the ROCK protein kinase from the PDK1 protein kinase. Advantageously, the agent dissociating the ROCK-PDK1 protein kinase complex is an intercalating agent capable of dissociating the ROCK-PDK1 protein kinase complex of an immune cell, fixing itself in the zone of interaction between the ROCK-PDK1 protein kinases, leading to a steric clash and separating the ROCK protein kinase from the PDK1 protein kinase.

Within the meaning of the present invention, by “combination” is meant a mixture of at least two inhibitors of the ROCK-PDK1 protein kinase complex, said inhibitors of the ROCK-PDK1 protein kinase complex being capable of being different inhibitors but belonging to the same class of inhibitors (for example: inhibitors of the ROCK protein kinase, inhibitors of the PDK1 protein kinase, or agents dissociating the ROCK-PDK1 protein kinase complex) or inhibitors belonging to different classes of inhibitors.

In a particular embodiment, the inhibitor of the ROCK-PDK1 protein kinase complex according to the invention can be a combination of at least two different inhibitors of the ROCK protein kinase, advantageously at least three different inhibitors of the ROCK protein kinase, advantageously at least four different inhibitors of the ROCK protein kinase, advantageously at least five different inhibitors of the ROCK protein kinase, advantageously at least six different inhibitors of the ROCK protein kinase, or more.

In another particular embodiment, the inhibitor of the ROCK-PDK1 protein kinase complex according to the invention can be a combination of at least two different inhibitors of the PDK1 protein kinase, advantageously at least three different inhibitors of the PDK1 protein kinase, advantageously at least four different inhibitors of the PDK1 protein kinase, advantageously at least five different inhibitors of the PDK1 protein kinase, advantageously at least six different inhibitors of the PDK1 protein kinase, or more.

In another particular embodiment, the inhibitor of the ROCK-PDK1 protein kinase complex according to the invention can be a combination of at least two different agents dissociating the ROCK-PDK1 protein kinase complex, advantageously at least three different agents dissociating the ROCK-PDK1 protein kinase complex, advantageously at least four different agents dissociating the ROCK-PDK1 protein kinase complex, advantageously at least five different agents dissociating the ROCK-PDK1 protein kinase complex, advantageously at least six different agents dissociating the ROCK-PDK1 protein kinase complex, or more.

In another particular embodiment, the inhibitor of the ROCK-PDK1 protein kinase complex according to the invention can be a combination of at least one inhibitor of the ROCK protein kinase and at least one inhibitor of the PDK1 protein kinase.

In another particular embodiment, the inhibitor of the ROCK-PDK1 protein kinase complex according to the invention can be a combination of at least one inhibitor of the ROCK protein kinase and at least one agent dissociating the ROCK-PDK1 protein kinase complex.

In another particular embodiment, the inhibitor of the ROCK-PDK1 protein kinase complex according to the invention can be a combination of at least one inhibitor of the PDK1 protein kinase and at least one agent dissociating the ROCK-PDK1 protein kinase complex.

In another particular embodiment, the inhibitor of the ROCK-PDK1 protein kinase complex according to the invention can be a combination of at least one inhibitor of the ROCK protein kinase, at least one inhibitor of the PDK1 protein kinase and at least one agent dissociating the ROCK-PDK1 protein kinase complex.

In an advantageous embodiment of the invention, the inhibitor of the ROCK-PDK1 protein kinase complex is selected from the compounds of formula (I) to (XIV):

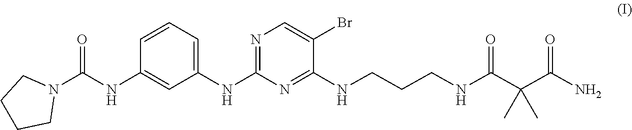 [SMILES: CC(C)(C(N)=O)C(=O)NCCCNc1nc(Nc2cccc(NC(=O)N3CCCC3)c2)ncc1Br]

or one of the pharmaceutically acceptable salts thereof

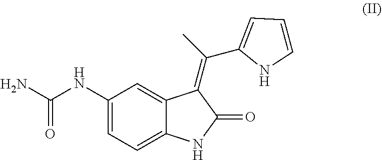 [SMILES: C/C(=C1/C(=O)Nc2ccc(NC(N)=O)cc21)c1ccc[nH]1]

or one of the pharmaceutically acceptable salts thereof.

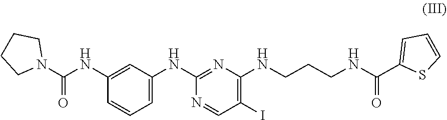 [SMILES: O=C(NCCCNc1nc(Nc2cccc(NC(=O)N3CCCC3)c2)ncc1I)c1cccs1]

or one of the pharmaceutically acceptable salts thereof

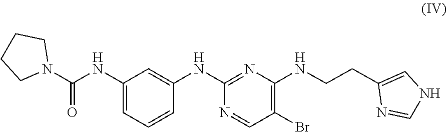 [SMILES: O=C(Nc1cccc(Nc2ncc(Br)c(NCCc3c[nH]cn3)n2)c1)N1CCCC1]

or one of the pharmaceutically acceptable salts thereof

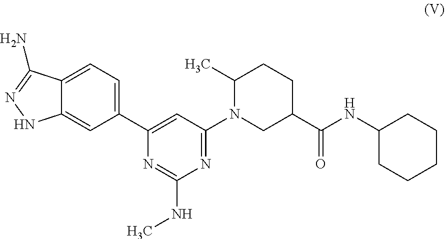 [SMILES: CNc1nc(-c2ccc3c(N)n[nH]c3c2)cc(N2CC(C(=O)NC3CCCCC3)CCC2C)n1]

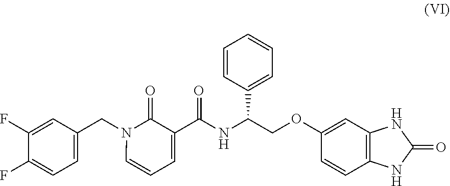 [SMILES: O=C(N[C@@H](COc1ccc2[nH]c(=O)[nH]c2c1)c1ccccc1)c1cccn(Cc2ccc(F)c(F)c2)c1=O]

or one of the pharmaceutically acceptable salts thereof.

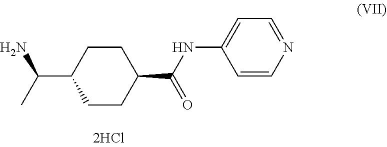 [SMILES: C[C@@H](N)[C@H]1CC[C@H](C(=O)Nc2ccncc2)CC1]

or one of the pharmaceutically acceptable salts thereof

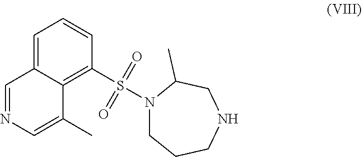 [SMILES: Cc1cncc2cccc(S(=O)(=O)N3CCCNCC3C)c12]

or one of the pharmaceutically acceptable salts thereof,

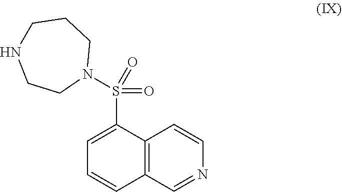 [SMILES: O=S(=O)(c1cccc2cnccc12)N1CCCNCC1]

or one of the pharmaceutically acceptable salts thereof,

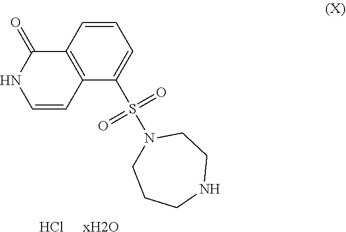 [SMILES: O=c1[nH]ccc2c(S(=O)(=O)N3CCCNCC3)cccc12 ; Cl ; "xH2O"]

or one of the pharmaceutically acceptable salts thereof,

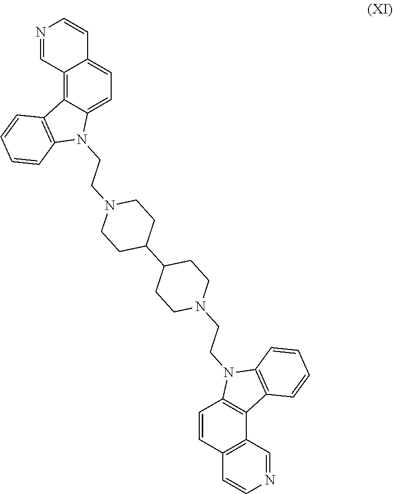 [SMILES: c1ccc2c(c1)c1c3cnccc3ccc1n2CCN1CCC(C2CCN(CCn3c4ccccc4c4c5cnccc5ccc43)CC2)CC1]

or one of the pharmaceutically acceptable salts thereof,

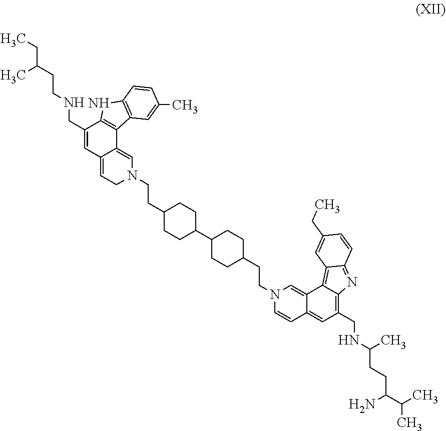 [SMILES: CCc1ccc2nc3c(CNC(C)CCC(N)C(C)C)cc4ccn(CCC5CCC(C6CCC(CCN7C=c8c(cc(CNCCC(C)CC)c9[nH]c%10ccc(C)cc%10c89)=CC7)CC6)CC5)cc4c3c2c1]

or one of the pharmaceutically acceptable salts thereof,

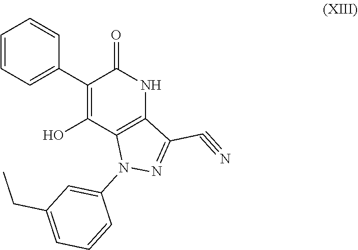 [SMILES: CCc1cccc(-n2nc(C#N)c3[nH]c(=O)c(-c4ccccc4)c(O)c32)c1]

or one of the pharmaceutically acceptable salts thereof,

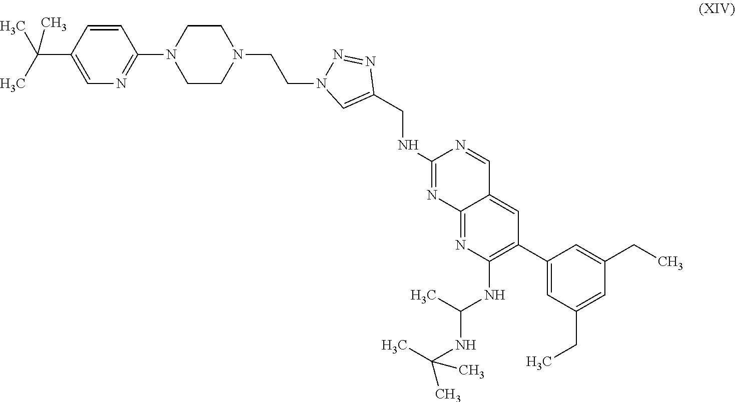 [SMILES: CCc1cc(CC)cc(-c2cc3cnc(NCc4cn(CCN5CCN(c6ccc(C(C)(C)C)cn6)CC5)nn4)nc3nc2NC(C)NC(C)(C)C)c1]

or one of the pharmaceutically acceptable salts thereof.

Advantageously, the compounds of formulae (I) to (VI) are inhibitors of the PDK1 protein kinase.

Advantageously, the compound of formula (I) is the compound BX320. Advantageously, the compound of formula (II) is the compound BX517. Advantageously, the compound of formula (III) is the compound BX795. Advantageously, the compound of formula (IV) is the compound BX912. Advantageously, the compound of formula (V) is the compound GSK2334470. Advantageously, the compound of formula (VI) is the compound MP7.

Advantageously, the compounds of formulae (VII) to (X) are inhibitors of the ROCK protein kinase.

Advantageously, the compound of formula (VII) is the compound Y-27632 (or Y27632).

Advantageously, the compound of formula (VIII) is dimethylfasudil. Advantageously, the compound of formula (IX) is fasudil. Advantageously, the compound of formula (X) is hydroxyfasudil.

Advantageously, the compounds of formulae (XI) to (XIV) are agents dissociating the ROCK-PDK1 protein kinase complex.

Advantageously, the inventors have shown that inhibition of the ROCK protein kinase by the compound of formula (VII) or the compound of formula (VIII) re-establishes a “normal” level of TNFα in the culture medium of the neurones infected by the prions. These results show that inhibition of the ROCK kinase opposes the production/secretion of a pro-inflammatory factor, TNFα.

Advantageously, the inventors have also shown that incubating human peripheral blood mononuclear cells with an inhibitor of the ROCK protein kinase selected from the compound of formula (VII) or the compound of formula (VIII), before being stimulated with an activator (R848) of the Toll-like receptors (TLR) ⅞ to mimic an infection with a single-stranded RNA virus, inhibits the production of TNFα induced by the TLR7/8 stimulation, but also the production and secretion of pro-inflammatory cytokines, such as IL-6, IL-1p and the interferons induced by the TLR7/8 stimulation.

Advantageously, the inventors have also shown that the inhibitor of the PDK1 protein kinase of formula (IV) inhibits the expression of the CD69 marker induced by the CD3/CD28 beads at the surface of the T lymphocytes, revealing an anti-inflammatory effect of the compound of formula (IV) on the activation of the T lymphocytes without affecting the viability thereof.

Advantageously, the inventors have also shown that the inhibitor of the PDK1 protein kinase of formula (IV) inhibits the expression of the CD69 marker induced by the CpG-A, TLR9 ligand at the surface of the B lymphocytes, revealing an anti-inflammatory effect of the PDK1 inhibitor of formula (IV) on the activation of the B lymphocytes without affecting the viability thereof.

Advantageously, the inventors have also shown that incubating purified human monocytes with an inhibitor of the PDK1 protein kinase (compound of formula (IV)), before being stimulated with an activator of the Toll-like receptors (TLR) 2/4 to mimic a bacterial infection, inhibits the production of TNFα induced by the TLR 2/4 stimulation, but also the secretion of pro-inflammatory cytokines on the human peripheral blood mononuclear cells, such as IL-6, IL-1β, GM-CSF and the interferons induced by stimulation of the TLR4 by lipopolysaccharide (LPS) or the TLR2 by lipoteichoic acids, two bacterial agents inducing an inflammatory response.

Thus, the inventors have shown, quite surprisingly, that the use of an inhibitor of the ROCK-PDK1 protein kinase complex, in particular an inhibitor of the ROCK protein kinase or an inhibitor of the PDK1 protein kinase has a preventive effect on the increase in production of inflammatory cytokines induced by the stimulation of TLR 2/4 or TLR7/8.

Thus, the inventors have shown, quite surprisingly, that the use of an inhibitor of the ROCK-PDK1 protein kinase complex, in particular an inhibitor of the ROCK protein kinase or an inhibitor of the PDK1 protein kinase has a curative effect on the production of interferon induced by the stimulation of TLR 2/4 or TLR7/8.

The inhibitors of the ROCK-PDK1 protein kinase complex according to the invention can be used in the form of a pharmaceutically acceptable salt and can comprise salts, hydrates and solvates prepared according to the conventional methods, well known to a person skilled in the art. By way of examples, there may be mentioned in particular the salts derived from carboxylic acids, such as the carboxylates (COO—), but also the sulfonates (—SO3−), the phosphonates (PO32−), the quaternary ammoniums (NR4+) or the sulfonamines (SO2—NH3+). Advantageously, the suitable salts comprise pharmaceutically or physiologically acceptable acid addition salts. Advantageously, the suitable salts comprise acid addition salts formed with various pharmaceutically or physiologically acceptable free acids. Examples of acids can comprise, without being limited thereto, hydrochloric acid, bromic acid, sulfuric acid, phosphoric acid, citric acid, acetic acid, lactic acid, tartaric acid, fumaric acid, formic acid, propionic acid, oxalic acid, trifluoroacetic acid, methanesulfonic acid, benzenesulfonic acid, maleic acid, benzoic acid, gluconic acid, glycolic acid, succinic acid, 4-morpholineethanesulfonic acid, camphorsulfonic acid, nitrobenzenesulfonic acid, hydroxy-O-sulfonic acid, 4-toluenesulfonic acid, knife ruktu acid, embonic acid, glutamic acid, aspartic acid, etc.

In addition, pharmaceutically acceptable metal salts can be prepared by using bases. For example, alkali metal salts or alkaline earth metal salts can be obtained by dissolving the compound in an excess of alkali metal hydroxide or alkaline earth metal hydroxide solution, by filtering the non-dissolved salts of the compound and evaporating and drying the filtrate. Here, the sodium, potassium, lithium, caesium, magnesium or calcium salts are pharmaceutically suitable metal salts, but are not limited thereto. In addition, silver salts corresponding to the metal salts can be obtained by reacting alkali metals or alkaline earth metals with suitable silver salts (for example nitrates). There may also be mentioned the ammoniums (NH4+) or the amines in the form of ammonium such as diethylamine (EDTA), pyrrolidine, piperidine and pyridine.

In a particularly advantageous embodiment of the invention, the inhibitor of the ROCK-PDK1 protein kinase complex is the compound of formula (IV)

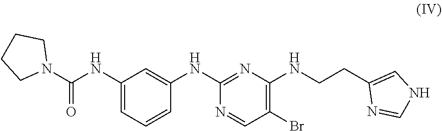 [SMILES: O=C(Nc1cccc(Nc2ncc(Br)c(NCCc3c[nH]cn3)n2)c1)N1CCCC1]

or a pharmaceutically acceptable salt thereof.

Another aspect of the present invention relates to a pharmaceutical composition comprising at least one inhibitor of the ROCK-PDK1 protein kinase complex as described above or a pharmaceutically acceptable salt thereof, as active principle and at least one pharmaceutically acceptable excipient and/or a carrier and/or a diluent and/or a pharmaceutically acceptable vehicle. Advantageously, at least one inhibitor of the ROCK-PDK1 protein kinase complex is selected from an inhibitor of the ROCK protein kinase, an inhibitor of the PDK1 protein kinase, an agent dissociating the ROCK-PDK1 protein kinase complex or a combination thereof.

In a particular embodiment, the pharmaceutical composition comprises at least one inhibitor of ROCK protein kinases as described above or a pharmaceutically acceptable salt thereof, as active principle and at least one pharmaceutically acceptable excipient and/or a carrier and/or a diluent and/or a pharmaceutically acceptable vehicle. Advantageously, the pharmaceutical composition according to the invention can comprise a combination of at least two different inhibitors of the ROCK protein kinase, advantageously at least three different inhibitors of the ROCK protein kinase, advantageously at least four different inhibitors of the ROCK protein kinase, advantageously at least five different inhibitors of the ROCK protein kinase, advantageously at least six different inhibitors of the ROCK protein kinase, or more.

In another particular embodiment, the pharmaceutical composition comprises at least one inhibitor of PDK1 protein kinases as described above or a pharmaceutically acceptable salt thereof, as active principle and at least one pharmaceutically acceptable excipient and/or a carrier and/or a diluent and/or a pharmaceutically acceptable vehicle. Advantageously, the pharmaceutical composition according to the invention can comprise a combination of at least two different inhibitors of the PDK1 protein kinase, advantageously at least three different inhibitors of the PDK1 protein kinase, advantageously at least four different inhibitors of the PDK1 protein kinase, advantageously at least five different inhibitors of the PDK1 protein kinase, advantageously at least six different inhibitors of the PDK1 protein kinase, or more.

In another particular embodiment, the pharmaceutical composition comprises at least one agent dissociating the ROCK-PDK1 protein kinase complex as described above or a pharmaceutically acceptable salt thereof, as active principle and at least one pharmaceutically acceptable excipient and/or a carrier and/or a diluent and/or a pharmaceutically acceptable vehicle. Advantageously, the pharmaceutical composition according to the invention can comprise a combination of at least two different agents dissociating the ROCK-PDK1 protein kinase complex, advantageously at least three different agents dissociating the ROCK-PDK1 protein kinase complex, advantageously at least four different agents dissociating the ROCK-PDK1 protein kinase complex, advantageously at least five different agents dissociating the ROCK-PDK1 protein kinase complex, advantageously at least six different agents dissociating the ROCK-PDK1 protein kinase complex, or more.

In another particular embodiment, the pharmaceutical composition comprises a combination comprising at least one inhibitor of ROCK protein kinases and at least one inhibitor of PDK1 protein kinases or one of the pharmaceutically acceptable salts thereof as described above, as active principle and at least one pharmaceutically acceptable excipient and/or a carrier and/or a diluent and/or a pharmaceutically acceptable vehicle.

In another particular embodiment, the pharmaceutical composition comprises a combination comprising at least one inhibitor of ROCK protein kinases and at least one agent dissociating the ROCK-PDK1 protein kinase complex or one of the pharmaceutically acceptable salts thereof as described above, as active principle and at least one pharmaceutically acceptable excipient and/or a carrier and/or a diluent and/or a pharmaceutically acceptable vehicle.

In another particular embodiment, the pharmaceutical composition comprises a combination comprising at least one inhibitor of PDK1 protein kinases and at least one agent dissociating the ROCK-PDK1 protein kinase complex or one of the pharmaceutically acceptable salts thereof as described above, as active principle and at least one pharmaceutically acceptable excipient and/or a carrier and/or a diluent and/or a pharmaceutically acceptable vehicle.

In another particular embodiment, the pharmaceutical composition comprises a combination comprising at least one inhibitor of ROCK protein kinases, at least one inhibitor of PDK1 protein kinases and at least one agent dissociating the ROCK-PDK1 protein kinase complex or one of the pharmaceutically acceptable salts thereof as described above, as active principle and at least one pharmaceutically acceptable excipient and/or a carrier and/or a diluent and/or a pharmaceutically acceptable vehicle.

In a particular embodiment, the invention relates to a pharmaceutical composition comprising a pharmaceutically active amount of at least one inhibitor of ROCK protein kinases or a pharmaceutically acceptable salt thereof as described above and at least one pharmaceutically acceptable excipient and/or a carrier and/or a diluent and/or a pharmaceutically acceptable vehicle. Advantageously, the pharmaceutical composition according to the invention can comprise a combination of at least two different inhibitors of the ROCK protein kinase, advantageously at least three different inhibitors of the ROCK protein kinase, advantageously at least four different inhibitors of the ROCK protein kinase, advantageously at least five different inhibitors of the ROCK protein kinase, advantageously at least six different inhibitors of the ROCK protein kinase, or more.

In another particular embodiment, the invention relates to a pharmaceutical composition comprising a pharmaceutically active amount of at least one inhibitor of PDK1 protein kinases or a pharmaceutically acceptable salt thereof as described above and at least one pharmaceutically acceptable excipient and/or a carrier and/or a diluent and/or a pharmaceutically acceptable vehicle.

Advantageously, the pharmaceutical composition according to the invention can comprise a combination of at least two different inhibitors of the PDK1 protein kinase, advantageously at least three different inhibitors of the PDK1 protein kinase, advantageously at least four different inhibitors of the PDK1 protein kinase, advantageously at least five different inhibitors of the PDK1 protein kinase, advantageously at least six different inhibitors of the PDK1 protein kinase, or more.

In another particular embodiment, the invention relates to a pharmaceutical composition comprising a pharmaceutically active amount of at least one agent dissociating the ROCK-PDK1 protein kinase complex or a pharmaceutically acceptable salt thereof as described above and at least one pharmaceutically acceptable excipient and/or a carrier and/or a diluent and/or a pharmaceutically acceptable vehicle. Advantageously, the pharmaceutical composition according to the invention can comprise a combination of at least two different agents dissociating the ROCK-PDK1 protein kinase complex, advantageously at least three different agents dissociating the ROCK-PDK1 protein kinase complex, advantageously at least four different agents dissociating the ROCK-PDK1 protein kinase complex, advantageously at least five different agents dissociating the ROCK-PDK1 protein kinase complex, advantageously at least six different agents dissociating the ROCK-PDK1 protein kinase complex, or more.

In another particular embodiment, the invention relates to a pharmaceutical composition comprising a pharmaceutically active amount of a combination comprising at least one inhibitor of ROCK protein kinases and at least one inhibitor of PDK1 protein kinases as described above or one of the pharmaceutically acceptable salts thereof and at least one pharmaceutically acceptable excipient and/or a carrier and/or a diluent and/or a pharmaceutically acceptable vehicle.

In another particular embodiment, the invention relates to a pharmaceutical composition comprising a pharmaceutically active amount of a combination comprising at least one inhibitor of ROCK protein kinases and at least one agent dissociating the ROCK-PDK1 protein kinase complex as described above or one of the pharmaceutically acceptable salts thereof and at least one pharmaceutically acceptable excipient and/or a carrier and/or a diluent and/or a pharmaceutically acceptable vehicle.

In another particular embodiment, the invention relates to a pharmaceutical composition comprising a pharmaceutically active amount of a combination comprising at least one inhibitor of PDK1 protein kinases and at least one agent dissociating the ROCK-PDK1 protein kinase complex as described above or one of the pharmaceutically acceptable salts thereof and at least one pharmaceutically acceptable excipient and/or a carrier and/or a diluent and/or a pharmaceutically acceptable vehicle.

In another particular embodiment, the invention relates to a pharmaceutical composition comprising a pharmaceutically active amount of a combination comprising at least one inhibitor of ROCK protein kinases, at least one inhibitor of PDK1 protein kinases and at least one agent dissociating the ROCK-PDK1 protein kinase complex as described above or one of the pharmaceutically acceptable salts thereof and at least one pharmaceutically acceptable excipient and/or a carrier and/or a diluent and/or a pharmaceutically acceptable vehicle.

In a particularly advantageous embodiment of the present invention, the pharmaceutical composition comprising the at least one inhibitor of the ROCK-PDK1 protein kinase complex as defined above is particularly efficacious for the prevention and/or the treatment of inflammatory diseases, viral infections and autoimmune diseases.

In a particularly advantageous embodiment of the present invention, the pharmaceutical composition comprising the at least one inhibitor of ROCK protein kinases or a pharmaceutically acceptable salt thereof as defined above is particularly efficacious for the prevention and/or the treatment of inflammatory diseases, viral infections and autoimmune diseases. Advantageously, the pharmaceutical composition according to the invention can comprise a combination of at least two different inhibitors of the ROCK protein kinase, advantageously at least three different inhibitors of the ROCK protein kinase, advantageously at least four different inhibitors of the ROCK protein kinase, advantageously at least five different inhibitors of the ROCK protein kinase, advantageously at least six different inhibitors of the ROCK protein kinase, or more.

In a particularly advantageous embodiment of the present invention, the pharmaceutical composition comprising the at least one inhibitor of PDK1 protein kinases or a pharmaceutically acceptable salt thereof as defined above is particularly efficacious for the prevention and/or the treatment of inflammatory diseases, viral infections and autoimmune diseases. Advantageously, the pharmaceutical composition according to the invention can comprise a combination of at least two different inhibitors of the PDK1 protein kinase, advantageously at least three different inhibitors of the PDK1 protein kinase, advantageously at least four different inhibitors of the PDK1 protein kinase, advantageously at least five different inhibitors of the PDK1 protein kinase, advantageously at least six different inhibitors of the PDK1 protein kinase, or more.

In a particularly advantageous embodiment of the present invention, the pharmaceutical composition comprising the at least one agent dissociating the ROCK-PDK1 protein kinase complex or a pharmaceutically acceptable salt thereof as defined above is particularly efficacious for the prevention and/or the treatment of inflammatory diseases, viral infections and autoimmune diseases.

Advantageously, the pharmaceutical composition according to the invention can comprise a combination of at least two different agents dissociating the ROCK-PDK1 protein kinase complex, advantageously at least three different agents dissociating the ROCK-PDK1 protein kinase complex, advantageously at least four different agents dissociating the ROCK-PDK1 protein kinase complex, advantageously at least five different agents dissociating the ROCK-PDK1 protein kinase complex, advantageously at least six different agents dissociating the ROCK-PDK1 protein kinase complex, or more.

In a particularly advantageous embodiment of the present invention, the pharmaceutical composition comprising the combination comprising at least one inhibitor of ROCK protein kinases and at least one inhibitor of PDK1 protein kinases or one of the pharmaceutically acceptable salts thereof, as described above, is particularly efficacious for the prevention and/or the treatment of inflammatory diseases, viral infections and autoimmune diseases.

In a particularly advantageous embodiment of the present invention, the pharmaceutical composition comprising the combination comprising at least one inhibitor of ROCK protein kinases and at least one agent dissociating the ROCK-PDK1 protein kinase complex or one of the pharmaceutically acceptable salts thereof, as described above, is particularly efficacious for the prevention and/or the treatment of inflammatory diseases, viral infections and autoimmune diseases.

In a particularly advantageous embodiment of the present invention, the pharmaceutical composition comprising the combination comprising at least one inhibitor of PDK1 protein kinases and at least one agent dissociating the ROCK-PDK1 protein kinase complex or one of the pharmaceutically acceptable salts thereof, as described above, is particularly efficacious for the prevention and/or the treatment of inflammatory diseases, viral infections and autoimmune diseases.

In a particularly advantageous embodiment of the present invention, the pharmaceutical composition comprising the combination comprising at least one inhibitor of ROCK protein kinases, at least one inhibitor of PDK1 protein kinases and at least one agent dissociating the ROCK-PDK1 protein kinase complex, or one of the pharmaceutically acceptable salts thereof, as described above, is particularly efficacious for the prevention and/or the treatment of inflammatory diseases, viral infections and autoimmune diseases.

In an embodiment according to the invention, the patient can be a human being. In another embodiment of the invention, the patient can be a non-human animal, in particular a dog, a cat, a horse, a cow, a pig, a sheep, a goat, a cervine animal or a primate.

According to embodiments that involve administration to a patient in need of a treatment, a therapeutically efficacious amount of at least one inhibitor of the ROCK-PDK1 protein kinase complex according to the invention, given herein as “therapeutically efficacious”, or “an efficacious amount for treatment”, or “pharmaceutically efficacious”, is the amount of inhibitor of the ROCK-PDK1 protein kinase complex or of a composition necessary to inhibit or reverse a disease (for example, for treating inflammation, viral infections and autoimmune diseases). Determining a therapeutically efficacious amount depends specifically on factors such as the toxicity and efficacy of the medicament. These factors will differ as a function of other factors such as the potency, the relative bioavailability, the bodyweight of the patient, the severity of the adverse effects and the preferred administration route.

The toxicity can be determined by using methods that are well known in the art. The efficacy can be determined by using the same directions. The efficacy can be measured by a reduction in inflammation or infection, for example in a juvenile idiopathic arthritis model. A pharmaceutically efficacious amount is therefore an amount that the clinician considers as toxicologically tolerable but efficacious.

The dosage can be suitably adjusted to obtain the desired medicament (for example, an inhibitor of the ROCK-PDK1 protein kinase complex of the invention) at localized or systemic levels as a function of the administration route. In the case where the patient response is insufficient at such doses, even higher doses (or higher efficacious doses via a different and more localized administration route) can be used inasmuch as patient tolerance permits. Multiple doses per day can also be used to reach suitable systemic levels of the inhibitor of the ROCK-PDK1 protein kinase complex according to the invention. Suitable systemic levels can be determined, for example, by measuring the maximum or prolonged plasma level of the patient. “Dose” and “dosage” are used interchangeably herein.

In an advantageous embodiment of the invention, the amount of inhibitor of the ROCK-PDK1 protein kinase complex according to the invention or of pharmaceutical composition comprising at least one inhibitor of the ROCK-PDK1 protein kinase complex as active principle administered to a patient is from 0.5 mg to 30 mg per kg of bodyweight, as a function of the severity of the inflammation.

Advantageously, the amount of inhibitor of the ROCK-PDK1 protein kinase complex according to the invention or of pharmaceutical composition comprising at least one inhibitor of the ROCK-PDK1 protein kinase complex as active principle administered to a patient is 1.0 mg, advantageously 1.5 mg, advantageously 2.0 mg, advantageously 2.5 mg, advantageously 3.0 mg, advantageously 3.5 mg, advantageously 4.0 mg, advantageously 4.5 mg, advantageously 5.0 mg, advantageously 5.5 mg, advantageously 6.0 mg, advantageously 6.5 mg, advantageously 7.0 mg, advantageously 7.5 mg, advantageously 8.0 mg, advantageously 8.5 mg, advantageously 9.0 mg, advantageously 9.5 mg, advantageously 10.0 mg, advantageously 10.5 mg, advantageously 11.0 mg, advantageously 11.5 mg, advantageously 12.0 mg, advantageously 12.5 mg, advantageously 13.0 mg, advantageously 13.5 mg, advantageously 14.0 mg, advantageously 14.5 mg, advantageously 15.0 mg, advantageously 15.5 mg, advantageously 16.0 mg, advantageously 16.5 mg, advantageously 17.0 mg, advantageously 17.5 mg, advantageously 18.0 mg, advantageously 18.5 mg, advantageously 19.0 mg, advantageously 19.5 mg, advantageously 20.0 mg, advantageously 20.5 mg, advantageously 21.0 mg, advantageously 21.5 mg, advantageously 22.0 mg, advantageously 22.5 mg, advantageously 23.0 mg, advantageously 23.5 mg, advantageously 24.0 mg, advantageously 24.5 mg, advantageously 25.0 mg, advantageously 25.5 mg, advantageously 26.0 mg, advantageously 26.5 mg, advantageously 27.0 mg, advantageously 27.5 mg, advantageously 28.0 mg, advantageously 28.5 mg, advantageously 29.0 mg, advantageously 29.5 mg, advantageously 30.0 mg per kg of bodyweight, as a function of the severity of the inflammation.

Advantageously, the amount of inhibitor of the ROCK-PDK1 protein kinase complex according to the invention or of pharmaceutical composition comprising at least one inhibitor of the ROCK-PDK1 protein kinase complex as active principle administered to a patient is comprised between 0.5 mg and 30.0 mg per kg of bodyweight, advantageously between 0.5 mg and 29.0 mg per kg of bodyweight, advantageously between 0.5 mg and 28.0 mg per kg of bodyweight, advantageously between 0.5 mg and 27.0 mg per kg of bodyweight, advantageously between 0.5 mg and 26.0 mg per kg of bodyweight, advantageously between 0.5 mg and 25.0 mg per kg of bodyweight, advantageously between 0.5 mg and 24.0 mg per kg of bodyweight, advantageously between 0.5 mg and 23.0 mg per kg of bodyweight, advantageously between 0.5 mg and 22.0 mg per kg of bodyweight, advantageously between 0.5 mg and 21 mg per kg of bodyweight, advantageously between 0.5 mg and 20.0 mg per kg of bodyweight, advantageously between 0.5 mg and 19.0 mg per kg of bodyweight, advantageously between 0.5 mg and 18.0 mg per kg of bodyweight, advantageously between 0.5 mg and 17.0 mg per kg of bodyweight, advantageously between 0.5 mg and 16.0 mg per kg of bodyweight advantageously between 0.5 mg and 15.0 mg per kg of bodyweight, advantageously between 0.5 mg and 14.0 mg per kg of bodyweight advantageously between 0.5 mg and 13.0 mg per kg of bodyweight, advantageously between 0.5 mg and 12.0 mg per kg of bodyweight advantageously between 0.5 mg and 11.0 mg per kg of bodyweight, advantageously between 0.5 mg and 10.0 mg per kg of bodyweight, advantageously between 0.5 mg and 9.0 mg per kg of bodyweight, advantageously between 0.5 mg and 8.0 mg per kg of bodyweight, advantageously between 0.5 mg and 7.0 mg per kg of bodyweight, advantageously between 0.5 mg and 6.0 mg per kg of bodyweight, advantageously between 1.0 mg and 6.0 mg per kg of bodyweight, advantageously between 1.5 mg and 6.0 mg per kg of bodyweight, advantageously between 1.5 mg and 5.5 mg per kg of bodyweight, advantageously between 2.0 mg and 5.0 mg per kg of bodyweight, as a function of the severity of the inflammation.

In an advantageous embodiment of the invention, the amount of inhibitor of the ROCK-PDK1 protein kinase complex according to the invention or of pharmaceutical composition comprising at least one inhibitor of the ROCK-PDK1 protein kinase complex as active principle administered to a patient is from 0.5 mg to 30 mg per kg of bodyweight, as a function of the severity of the infection. Advantageously, the amount of inhibitor of the ROCK-PDK1 protein kinase complex according to the invention or of pharmaceutical composition comprising at least one inhibitor of the ROCK-PDK1 protein kinase complex as active principle administered to a patient is 1.0 mg, advantageously 1.5 mg, advantageously 2.0 mg, advantageously 2.5 mg, advantageously 3.0 mg, advantageously 3.5 mg, advantageously 4.0 mg, advantageously 4.5 mg, advantageously 5.0 mg, advantageously 5.5 mg, advantageously 6.0 mg, advantageously 6.5 mg, advantageously 7.0 mg, advantageously 7.5 mg, advantageously 8.0 mg, advantageously 8.5 mg, advantageously 9.0 mg, advantageously 9.5 mg, advantageously 10.0 mg, advantageously 10.5 mg, advantageously 11.0 mg, advantageously 11.5 mg, advantageously 12.0 mg, advantageously 12.5 mg, advantageously 13.0 mg, advantageously 13.5 mg, advantageously 14.0 mg, advantageously 14.5 mg, advantageously 15.0 mg, advantageously 15.5 mg, advantageously 16.0 mg, advantageously 16.5 mg, advantageously 17.0 mg, advantageously 17.5 mg, advantageously 18.0 mg, advantageously 18.5 mg, advantageously 19.0 mg, advantageously 19.5 mg, advantageously 20.0 mg, advantageously 20.5 mg, advantageously 21.0 mg, advantageously 21.5 mg, advantageously 22.0 mg, advantageously 22.5 mg, advantageously 23.0 mg, advantageously 23.5 mg, advantageously 24.0 mg, advantageously 24.5 mg, advantageously 25.0 mg, advantageously 25.5 mg, advantageously 26.0 mg, advantageously 26.5 mg, advantageously 27.0 mg, advantageously 27.5 mg, advantageously 28.0 mg, advantageously 28.5 mg, advantageously 29.0 mg, advantageously 29.5 mg, advantageously 30.0 mg per kg of bodyweight, as a function of the severity of the infection.

Advantageously, the amount of inhibitor of the ROCK-PDK1 protein kinase complex according to the invention or of pharmaceutical composition comprising at least one inhibitor of the ROCK-PDK1 protein kinase complex as active principle administered to a patient is comprised between 0.5 mg and 30.0 mg per kg of bodyweight, advantageously between 0.5 mg and 29.0 mg per kg of bodyweight, advantageously between 0.5 mg and 28.0 mg per kg of bodyweight, advantageously between 0.5 mg and 27.0 mg per kg of bodyweight, advantageously between 0.5 mg and 26.0 mg per kg of bodyweight, advantageously between 0.5 mg and 25.0 mg per kg of bodyweight, advantageously between 0.5 mg and 24.0 mg per kg of bodyweight, advantageously between 0.5 mg and 23.0 mg per kg of bodyweight, advantageously between 0.5 mg and 22.0 mg per kg of bodyweight, advantageously between 0.5 mg and 21 mg per kg of bodyweight, advantageously between 0.5 mg and 20.0 mg per kg of bodyweight, advantageously between 0.5 mg and 19.0 mg per kg of bodyweight, advantageously between 0.5 mg and 18.0 mg per kg of bodyweight, advantageously between 0.5 mg and 17.0 mg per kg of bodyweight, advantageously between 0.5 mg and 16.0 mg per kg of bodyweight advantageously between 0.5 mg and 15.0 mg per kg of bodyweight, advantageously between 0.5 mg and 14.0 mg per kg of bodyweight advantageously between 0.5 mg and 13.0 mg per kg of bodyweight, advantageously between 0.5 mg and 12.0 mg per kg of bodyweight advantageously between 0.5 mg and 11.0 mg per kg of bodyweight, advantageously between 0.5 mg and 10.0 mg per kg of bodyweight, advantageously between 0.5 mg and 9.0 mg per kg of bodyweight, advantageously between 0.5 mg and 8.0 mg per kg of bodyweight, advantageously between 0.5 mg and 7.0 mg per kg of bodyweight, advantageously between 0.5 mg and 6.0 mg per kg of bodyweight, advantageously between 1.0 mg and 6.0 mg per kg of bodyweight, advantageously between 1.5 mg and 6.0 mg per kg of bodyweight, advantageously between 1.5 mg and 5.5 mg per kg of bodyweight, advantageously between 2.0 mg and 5.0 mg per kg of bodyweight, as a function of the severity of the infection.

Of course, this unit dose can be repeated if necessary.

In an advantageous embodiment, the inhibitor of the ROCK-PDK1 protein kinase complex as active principle is administered to the patient at the rate of 100 mg to 500 mg per day. Advantageously, the inhibitor of the ROCK-PDK1 protein kinase complex as active principle is administered to the patient at the rate of 100 mg to 450 mg per day, advantageously at the rate of 100 mg to 400 mg per day, advantageously at the rate of 100 mg to 350 mg per day, advantageously at the rate of 100 mg to 300 mg per day, advantageously at the rate of 100 mg to 250 mg per day, advantageously at the rate of 100 mg to 200 mg per day, advantageously at the rate of 100 mg to 150 mg per day, advantageously at the rate of 140 mg per day. Of course, this unit dose can be repeated if necessary.

In an advantageous embodiment, the pharmaceutical composition according to the invention is administered to the patient at the rate of one, two, three, four, five, six or seven times per week.

Advantageously, the pharmaceutical composition according to the invention is administered to the patient at the rate of seven times per week, i.e. one administration per day during seven consecutive days. Advantageously, the pharmaceutical composition according to the invention is administered to the patient at the rate of six times per week. Advantageously, the pharmaceutical composition according to the invention is administered to the patient at the rate of five times per week.

Advantageously, the pharmaceutical composition according to the invention is administered to the patient at the rate of four times per week. Advantageously, the pharmaceutical composition according to the invention is administered to the patient at the rate of three times per week. Advantageously, the pharmaceutical composition according to the invention is administered to the patient at the rate of twice per week. Advantageously, the pharmaceutical composition according to the invention is administered to the patient at the rate of once per week.

In an advantageous embodiment of the invention, the pharmaceutical compositions supplied are used for in vivo applications. As a function of the intended administration route in vivo, the pharmaceutical compositions used can be in solid, semi-solid or liquid dosage form, such as for example tablets, pills, powders, capsules, gels, ointments, liquids, suspensions. Preferably, the pharmaceutical compositions are administered in unit dosage forms suitable for a single administration of precise doses. The pharmaceutical compositions can also comprise, as a function of the desired formulation, at least one pharmaceutically acceptable carrier or diluent, defined as aqueous-based vehicles habitually used for the formulation of pharmaceutical compositions for animal or human administration. The diluent is selected so as not to affect the biological activity of the inhibitor of the ROCK-PDK1 protein kinase complex according to the invention. Examples of such diluents are distilled water, physiological serum, Ringer's solution, dextrose solution and Hank's solution. In addition, the pharmaceutical composition can also comprise other medicinal agents, pharmaceutical agents, carriers, adjuvants, stabilizers that are non-toxic, non-therapeutic, non-immunogenic, etc. Efficacious amounts of such a diluent or vehicle are amounts that are efficacious for obtaining a pharmaceutically acceptable solution in terms of formulation, solubility of the components, biological activity, etc. In certain embodiments, the pharmaceutical compositions given herein are sterile.

It can be envisaged to formulate at least one inhibitor of the ROCK-PDK1 protein kinase complex according to the present invention at the rate of 0.5 to 98% by weight, expressed by weight, or even more, with respect to the total weight of the pharmaceutical composition in question.

In a particularly advantageous embodiment of the present invention, the pharmaceutical composition according to the invention comprises only at least one inhibitor of the ROCK-PDK1 protein kinase complex, as single active principle.

In a particular embodiment of the invention, the inhibitor of the ROCK-PDK1 protein kinase complex of the pharmaceutical composition is selected from the compounds of formula (I) to (XIV):

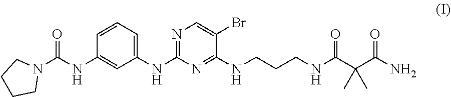 [SMILES: CC(C)(C(N)=O)C(=O)NCCCNc1nc(Nc2cccc(NC(=O)N3CCCC3)c2)ncc1Br]

or one of the pharmaceutically acceptable salts thereof

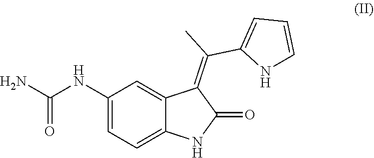 [SMILES: C/C(=C1/C(=O)Nc2ccc(NC(N)=O)cc21)c1ccc[nH]1]

or one of the pharmaceutically acceptable salts thereof.

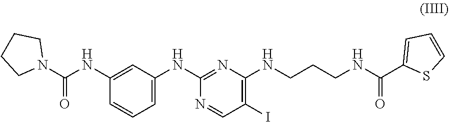 [SMILES: O=C(NCCCNc1nc(Nc2cccc(NC(=O)N3CCCC3)c2)ncc1I)c1cccs1]

or one of the pharmaceutically acceptable salts thereof

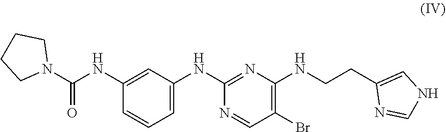 [SMILES: O=C(Nc1cccc(Nc2ncc(Br)c(NCCc3c[nH]cn3)n2)c1)N1CCCC1]

or one of the pharmaceutically acceptable salts thereof.

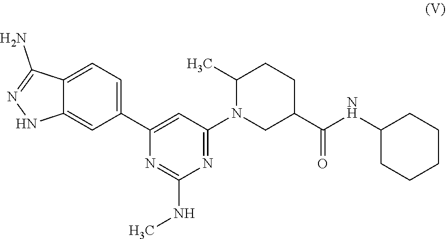 [SMILES: CNc1nc(-c2ccc3c(N)n[nH]c3c2)cc(N2CC(C(=O)NC3CCCCC3)CCC2C)n1]

or one of the pharmaceutically acceptable salts thereof

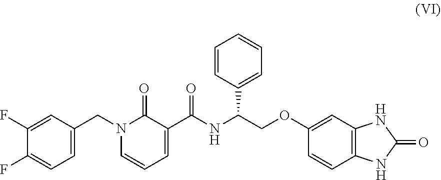 [SMILES: O=C(N[C@@H](COc1ccc2[nH]c(=O)[nH]c2c1)c1ccccc1)c1cccn(Cc2ccc(F)c(F)c2)c1=O]

or one of the pharmaceutically acceptable salts thereof.

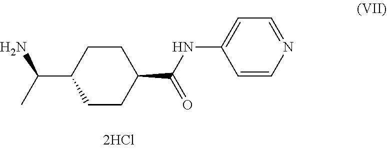 [SMILES: C[C@@H](N)[C@H]1CC[C@H](C(=O)Nc2ccncc2)CC1]

or one of the pharmaceutically acceptable salts thereof

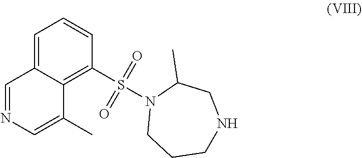 [SMILES: Cc1cncc2cccc(S(=O)(=O)N3CCCNCC3C)c12]

or one of the pharmaceutically acceptable salts thereof,

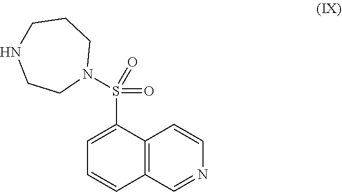 [SMILES: O=S(=O)(c1cccc2cnccc12)N1CCCNCC1]

or one of the pharmaceutically acceptable salts thereof,

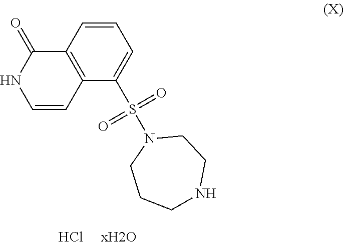 [SMILES: O=c1[nH]ccc2c(S(=O)(=O)N3CCCNCC3)cccc12 ; Cl]

or one of the pharmaceutically acceptable salts thereof,

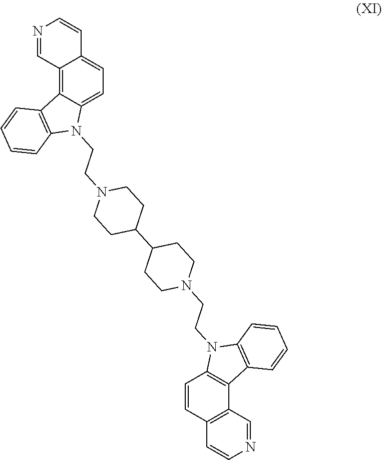 [SMILES: c1ccc2c(c1)c1c3cnccc3ccc1n2CCN1CCC(C2CCN(CCn3c4ccccc4c4c5cnccc5ccc43)CC2)CC1]

or one of the pharmaceutically acceptable salts thereof,

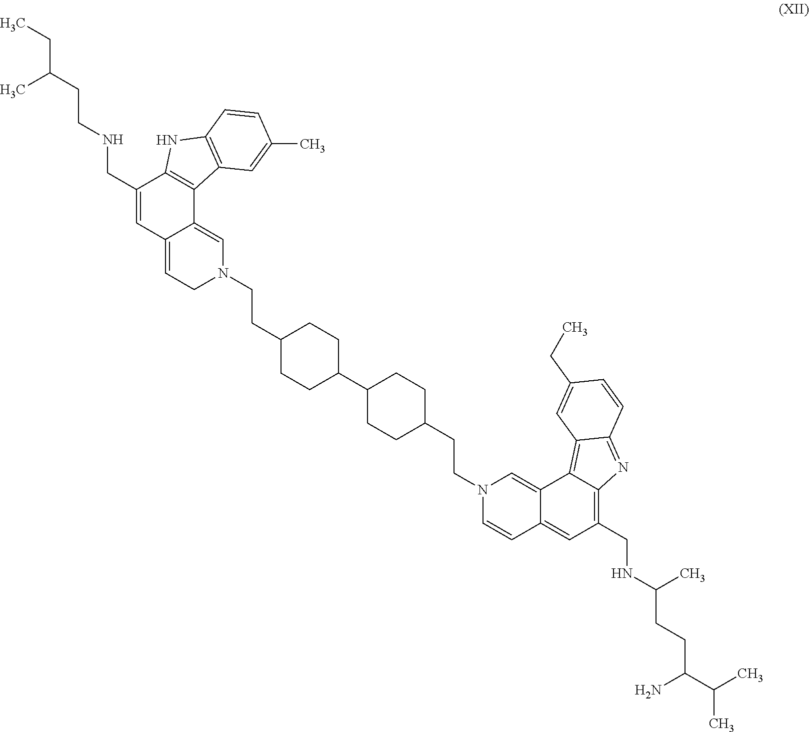 [SMILES: CCc1ccc2nc3c(CNC(C)CCC(N)C(C)C)cc4ccn(CCC5CCC(C6CCC(CCN7C=c8c(cc(CNCCC(C)CC)c9[nH]c%10ccc(C)cc%10c89)=CC7)CC6)CC5)cc4c3c2c1]

or one of the pharmaceutically acceptable salts thereof,

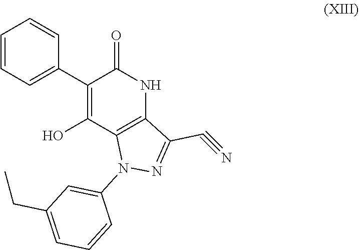 [SMILES: CCc1cccc(-n2nc(C#N)c3[nH]c(=O)c(-c4ccccc4)c(O)c32)c1]

or one of the pharmaceutically acceptable salts thereof.

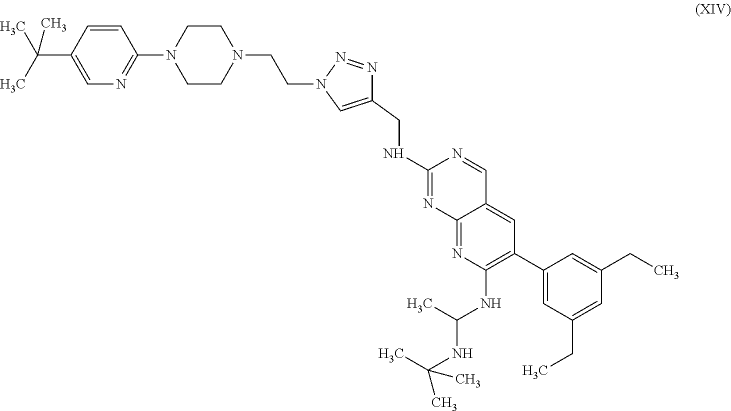 [SMILES: CCc1cc(CC)cc(-c2cc3cnc(NCc4cn(CCN5CCN(c6ccc(C(C)(C)C)cn6)CC5)nn4)nc3nc2NC(C)NC(C)(C)C)c1]

or one of the pharmaceutically acceptable salts thereof.

In a particularly advantageous embodiment of the invention, the pharmaceutical composition comprises the inhibitor of the ROCK-PDK1 protein kinase complex of formula (I) or a pharmaceutically acceptable salt thereof, as single active principle

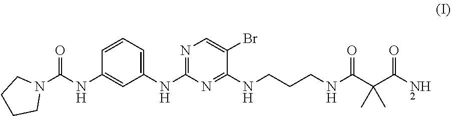 [SMILES: CC(C)(C(N)=O)C(=O)NCCCNc1nc(Nc2cccc(NC(=O)N3CCCC3)c2)ncc1Br]

or one of the pharmaceutically acceptable salts thereof and at least one pharmaceutically acceptable excipient and/or a carrier or a diluent or a pharmaceutically acceptable vehicle.

In a particularly advantageous embodiment of the invention, the pharmaceutical composition comprises the inhibitor of the ROCK-PDK1 protein kinase complex of formula (II) or a pharmaceutically acceptable salt thereof, as single active principle

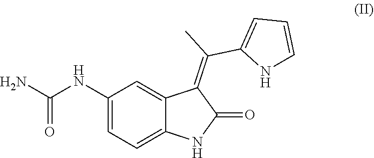 [SMILES: C/C(=C1/C(=O)Nc2ccc(NC(N)=O)cc21)c1ccc[nH]1]

or one of the pharmaceutically acceptable salts thereof and at least one pharmaceutically acceptable excipient and/or a carrier or a diluent or a pharmaceutically acceptable vehicle.

In a particularly advantageous embodiment of the invention, the pharmaceutical composition comprises the inhibitor of the ROCK-PDK1 protein kinase complex of formula (III) or a pharmaceutically acceptable salt thereof, as single active principle

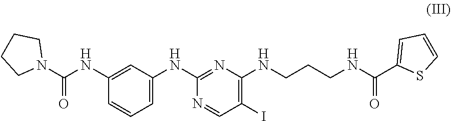 [SMILES: O=C(NCCCNc1nc(Nc2cccc(NC(=O)N3CCCC3)c2)ncc1I)c1cccs1]

or one of the pharmaceutically acceptable salts thereof and at least one pharmaceutically acceptable excipient and/or a carrier or a diluent or a pharmaceutically acceptable vehicle.

In a particularly advantageous embodiment of the invention, the pharmaceutical composition comprises the inhibitor of the ROCK-PDK1 protein kinase complex of formula (IV) or a pharmaceutically acceptable salt thereof, as single active principle

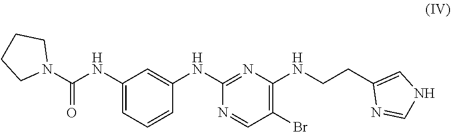 [SMILES: O=C(Nc1cccc(Nc2ncc(Br)c(NCCc3c[nH]cn3)n2)c1)N1CCCC1]

or one of the pharmaceutically acceptable salts thereof and at least one pharmaceutically acceptable excipient and/or a carrier or a diluent or a pharmaceutically acceptable vehicle.

In a particularly advantageous embodiment of the invention, the pharmaceutical composition comprises the inhibitor of the ROCK-PDK1 protein kinase complex of formula (V) or a pharmaceutically acceptable salt thereof, as single active principle

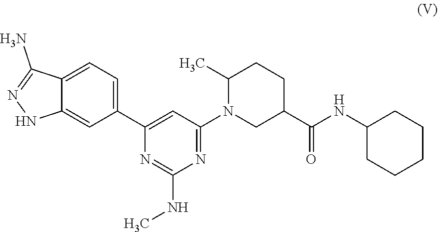 [SMILES: CNc1nc(-c2ccc3c(N)n[nH]c3c2)cc(N2CC(C(=O)NC3CCCCC3)CCC2C)n1]

or one of the pharmaceutically acceptable salts thereof and at least one pharmaceutically acceptable excipient and/or a carrier or a diluent or a pharmaceutically acceptable vehicle.

In a particularly advantageous embodiment of the invention, the pharmaceutical composition comprises the inhibitor of the ROCK-PDK1 protein kinase complex of formula (VI) or a pharmaceutically acceptable salt thereof, as single active principle

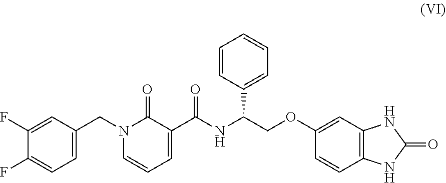 [SMILES: O=C(N[C@@H](COc1ccc2[nH]c(=O)[nH]c2c1)c1ccccc1)c1cccn(Cc2ccc(F)c(F)c2)c1=O]

or one of the pharmaceutically acceptable salts thereof and at least one pharmaceutically acceptable excipient and/or a carrier or a diluent or a pharmaceutically acceptable vehicle.

In a particularly advantageous embodiment of the invention, the pharmaceutical composition comprises the inhibitor of the ROCK-PDK1 protein kinase complex of formula (VII) or a pharmaceutically acceptable salt thereof, as single active principle

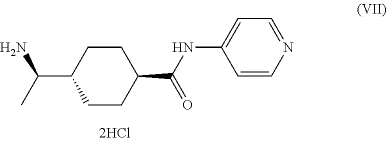 [SMILES: C[C@@H](N)[C@H]1CC[C@H](C(=O)Nc2ccncc2)CC1]

or one of the pharmaceutically acceptable salts thereof and at least one pharmaceutically acceptable excipient and/or a carrier or a diluent or a pharmaceutically acceptable vehicle.

In a particularly advantageous embodiment of the invention, the pharmaceutical composition comprises the inhibitor of the ROCK-PDK1 protein kinase complex of formula (VIII) or a pharmaceutically acceptable salt thereof, as single active principle

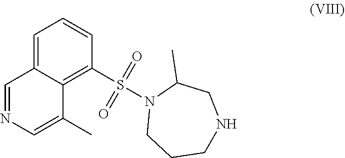 [SMILES: Cc1cncc2cccc(S(=O)(=O)N3CCCNCC3C)c12]

or one of the pharmaceutically acceptable salts thereof and at least one pharmaceutically acceptable excipient and/or a carrier or a diluent or a pharmaceutically acceptable vehicle.

In a particularly advantageous embodiment of the invention, the pharmaceutical composition comprises the inhibitor of the ROCK-PDK1 protein kinase complex of formula (IX) or a pharmaceutically acceptable salt thereof, as single active principle

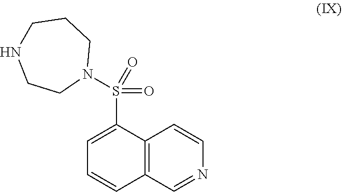 [SMILES: O=S(=O)(c1cccc2cnccc12)N1CCCNCC1]

or one of the pharmaceutically acceptable salts thereof and at least one pharmaceutically acceptable excipient and/or a carrier or a diluent or a pharmaceutically acceptable vehicle.

In a particularly advantageous embodiment of the invention, the pharmaceutical composition comprises the inhibitor of the ROCK-PDK1 protein kinase complex of formula (X) or a pharmaceutically acceptable salt thereof, as single active principle

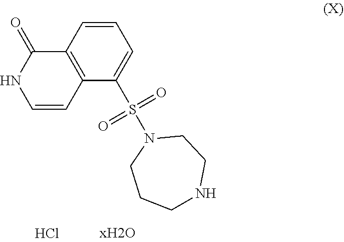 [SMILES: O=c1[nH]ccc2c(S(=O)(=O)N3CCCNCC3)cccc12 ; Cl]

or one of the pharmaceutically acceptable salts thereof and at least one pharmaceutically acceptable excipient and/or a carrier or a diluent or a pharmaceutically acceptable vehicle.

In a particularly advantageous embodiment of the invention, the pharmaceutical composition comprises the inhibitor of the ROCK-PDK1 protein kinase complex of formula (XI) or a pharmaceutically acceptable salt thereof, as single active principle

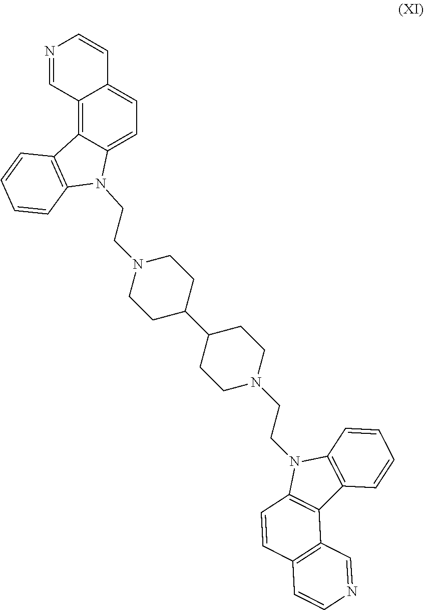 [SMILES: c1ccc2c(c1)c1c3cnccc3ccc1n2CCN1CCC(C2CCN(CCn3c4ccccc4c4c5cnccc5ccc43)CC2)CC1]

or one of the pharmaceutically acceptable salts thereof and at least one pharmaceutically acceptable excipient and/or a carrier or a diluent or a pharmaceutically acceptable vehicle.

In a particularly advantageous embodiment of the invention, the pharmaceutical composition comprises the inhibitor of the ROCK-PDK1 protein kinase complex of formula (XII) or a pharmaceutically acceptable salt thereof, as single active principle

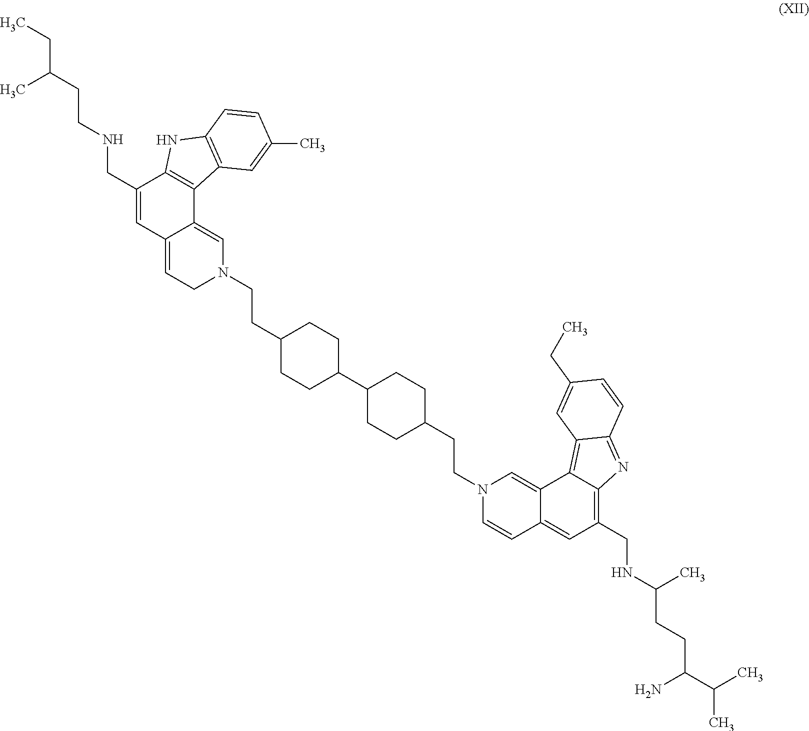 [SMILES: CCc1ccc2nc3c(CNC(C)CCC(N)C(C)C)cc4ccn(CCC5CCC(C6CCC(CCN7C=c8c(cc(CNCCC(C)CC)c9[nH]c%10ccc(C)cc%10c89)=CC7)CC6)CC5)cc4c3c2c1]

or one of the pharmaceutically acceptable salts thereof and at least one pharmaceutically acceptable excipient and/or a carrier or a diluent or a pharmaceutically acceptable vehicle.

In a particularly advantageous embodiment of the invention, the pharmaceutical composition comprises the inhibitor of the ROCK-PDK1 protein kinase complex of formula (XIII) or a pharmaceutically acceptable salt thereof, as single active principle

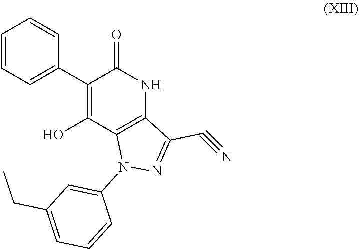 [SMILES: CCc1cccc(-n2nc(C#N)c3[nH]c(=O)c(-c4ccccc4)c(O)c32)c1]

or one of the pharmaceutically acceptable salts thereof and at least one pharmaceutically acceptable excipient and/or a carrier or a diluent or a pharmaceutically acceptable vehicle.

In a particularly advantageous embodiment of the invention, the pharmaceutical composition comprises the inhibitor of the ROCK-PDK1 protein kinase complex of formula (XIV) or a pharmaceutically acceptable salt thereof, as single active principle

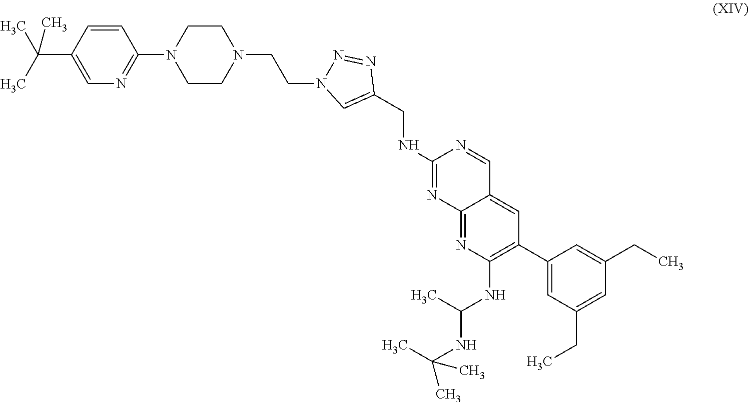 [SMILES: CCc1cc(CC)cc(-c2cc3cnc(NCc4cn(CCN5CCN(c6ccc(C(C)(C)C)cn6)CC5)nn4)nc3nc2NC(C)NC(C)(C)C)c1]

or one of the pharmaceutically acceptable salts thereof and at least one pharmaceutically acceptable excipient and/or a carrier or a diluent or a pharmaceutically acceptable vehicle.

In an advantageous embodiment, the pharmaceutical composition according to the invention also comprises at least one second active principle. In a particularly advantageous embodiment of the invention, the at least one second active principle can interact synergically with the inhibitor of the ROCK-PDK1 protein kinase complex according to the invention. Advantageously, the second active principle can be a corticosteroid, advantageously prednisone.

In an advantageous embodiment of the invention, when the pharmaceutical composition comprises a second active principle, the pharmaceutical composition is adapted for a simultaneous administration or sequential administration of the inhibitor of the ROCK-PDK1 protein kinase complex according to the invention and of the second active principle.

Within the meaning of the present invention, by “simultaneous administration” is meant an administration of the inhibitor of the ROCK-PDK1 protein kinase complex according to the invention and of the second active principle at the same time, at the same moment, to a patient in therapeutically efficacious amounts in order to allow the synergic effect of the pharmaceutical composition. Nevertheless this does not mean that the inhibitor of the ROCK-PDK1 protein kinase complex according to the invention and the second active principle are necessarily administered in the form of a mixture; they can indeed be administered simultaneously but separately, in the form of separate compositions.

By “present in two separate compositions” is meant that the inhibitor of the ROCK-PDK1 protein kinase complex according to the invention and the second active principle are physically separate. They are then utilized, administered separately in therapeutically efficacious amounts in order to allow the synergic effect of the pharmaceutical composition, without mixing beforehand, in several (at least two) dosage forms (for example two separate compositions).

In another particular embodiment of the invention, the inhibitor of the ROCK-PDK1 protein kinase complex according to the invention and the second active principle can be present in one and the same composition in therapeutically efficacious amounts in order to allow the synergic effect of the pharmaceutical composition. Within the meaning of the present invention, by “present in one and the same composition” is meant the physical combination of the inhibitor of the ROCK-PDK1 protein kinase complex according to the invention and the second active principle. The inhibitor of the ROCK-PDK1 protein kinase complex according to the invention and the second active principle are then necessarily administered simultaneously since they are administered together, in the form of a mixture, in one and the same dosage form (for example in one and the same composition containing genetically modified human stem cells and the antineoplastic agent) and in therapeutically efficacious amounts in order to allow the synergic effect of the pharmaceutical composition.

Within the meaning of the present invention, by “sequential administration” is meant that the inhibitor of the ROCK-PDK1 protein kinase complex according to the invention and the second active principle are administered in therapeutically efficacious amounts in order to allow the synergic effect of the pharmaceutical composition, not simultaneously but separately over time, one after the other. The terms “precede” or “preceding” and “follow” or “following” are then applied. The term “precede” or “preceding” is used when a compound of the pharmaceutical composition according to the invention is administered a few minutes or several hours, or even several days before the administration of the other compound(s) of the pharmaceutical composition. Conversely, the term “follow” or “following” is used when a compound of the pharmaceutical composition is administered a few minutes or several hours, or even several days after the administration of the other compound(s) of the pharmaceutical composition. In a particularly advantageous embodiment of the invention, the inhibitor of the ROCK-PDK1 protein kinase complex according to the invention is administered a few days before the second active principle.

In an advantageous embodiment of the invention, when the pharmaceutical composition comprises a second active principle, the pharmaceutical composition is adapted for a sequential administration of the inhibitor of the ROCK-PDK1 protein kinase complex according to the invention and of the second active principle. Advantageously, the second active principle can be a corticosteroid, advantageously prednisone.

In a particular embodiment of the invention, the inhibitor of the ROCK-PDK1 protein kinase complex according to the invention is administered to the patient, then two to three days after the administration of the inhibitor of the ROCK-PDK1 protein kinase complex according to the invention, the second active principle is administered to this same patient, for at least a week, advantageously at least two weeks, advantageously at least three weeks, advantageously at least four weeks, advantageously at least five weeks, advantageously at least six weeks, advantageously at least seven weeks.

The administration during the treatment in vivo can be carried out by any route whatsoever, including oral, parenteral, transdermal, intramuscular, intranasal, sublingual, intratracheal, by inhalation, ocular, vaginal and rectal. Intracapsular, intravenous and intraperitoneal administration routes can also be used. A person skilled in the art recognises that the administration route varies as a function of the disorder to be treated. For example, the pharmaceutical compositions according to the invention or the inhibitor of the ROCK-PDK1 protein kinase complex of the present invention can be administered to a patient by oral, parenteral or topical administration. In an embodiment, the pharmaceutical compositions according to the invention or the inhibitor of the ROCK-PDK1 protein kinase complex of the present invention are in a form adapted for administration thereof by the oral, parenteral or topical route. In an embodiment, the pharmaceutical compositions according to the invention or the inhibitor of the ROCK-PDK1 protein kinase complex of the present invention, optionally in the form of nanoparticles, are administered by intravenous perfusion.

When it is desirable to administer the pharmaceutical compositions by the systemic route, they can be formulated for a parenteral administration by injection, for example by bolus injection or continuous perfusion. The formulations for injection can be presented in a unit dosage form, for example in ampoules, or in multiple-dose recipients, with an added preservative. The pharmaceutical compositions can adopt forms such as suspensions, solutions or emulsions in oily or aqueous vehicles, and can contain formulation agents such as suspension agents, stabilizers and/or dispersing agents.

The pharmaceutical formulations for parenteral administration comprise the aqueous solutions of the active pharmaceutical compositions in water-soluble form. In addition, suspensions of the active pharmaceutical compositions can be prepared in the form of suitable suspensions for oily injection.

The suitable lipophilic vehicles or solvents comprise fatty oils such as sesame oil, or synthetic fatty acid esters, such as ethyl oleate or triglycerides, or liposomes. The aqueous suspensions for injection can contain substances that increase the viscosity of the suspension, such as sodium carboxymethylcellulose, sorbitol or dextran.

Optionally, the suspension can also contain stabilizers or suitable agents that increase the solubility of the pharmaceutical compositions in order to allow the preparation of highly concentrated solutions.

In a variant, the active compositions can be in powder form in order to constitute a suitable vehicle, for example sterile non-pyrogenic water, before use.

For oral administration, the pharmaceutical compositions can adopt the form, for example, of tablets or capsules prepared by conventional means with pharmaceutically acceptable excipients such as binding agents (for example pregelatinized maize starch, polyvinylpyrrolidone, methyl cellulose or hydroxypropyl methyl cellulose); fillers (for example lactose, microcrystalline cellulose or calcium hydrogenophosphate); lubricants (for example magnesium stearate, talc or silica); disintegrating agents (for example potato starch or sodium starch glycolate); or wetting agents (for example sodium laurylsulfate). The tablets can be coated by methods that are well known in the art. The liquid preparations for oral administration can adopt the form, for example, of solutions, syrups or suspensions, or can be presented in the form of dry product for reconstitution with water or another suitable vehicle before use. These liquid preparations can be prepared by conventional means with pharmaceutically acceptable additives such as suspension agents (for example sorbitol syrup, cellulose derivatives or edible hydrogenated fats); emulsifying agents (for example lecithin or gum arabic); non-aqueous vehicles (for example almond oil, oily esters, ethyl alcohol or fractionated vegetable oils); and preservatives (for example methyl or propyl-p-hydroxybenzoates or sorbic acid). The preparations can also contain buffer salts, flavourings, colorants and sweeteners, as appropriate. It is also desirable to increase the overall stability of the compounds of formula (I) as defined and increase the circulation time in the body. Examples of such molecules comprise: polyethylene glycol, ethylene glycol and propylene glycol copolymers, carboxymethyl cellulose, dextran, polyvinyl alcohol, polyvinyl pyrrolidone and polyproline. Other polymers capable of being used are poly-1,3-dioxolane and poly-1,3,6-tioxocane. As indicated above, the polyethylene glycol molecules are preferred for pharmaceutical use. For the oral compositions, the place of release can be the stomach, the small intestine (duodenum, jejunum, or ileum) or the large intestine. A person skilled in the art will have available formulations that will not dissolve in the stomach, but which will release the substance in the duodenum or elsewhere in the intestine. Preferably, the release will avoid the deleterious effects of the stomach environment by release of the biologically active substance beyond the stomach environment, such as in the intestine.

For oral administration, the compositions can adopt the form of tablets or lozenges formulated in conventional manner.

For an administration by inhalation, the compositions to be used according to the present invention can be delivered in a suitable manner in the form of an aerosol spray based on pressurized packaging or a nebulizer, by using a suitable propellant, for example dichlorodifluoromethane, trichlorofluoromethane, dichlorotetrafluoroethane, carbon dioxide or other suitable gas. In the case of a pressurized aerosol, the dosage unit can be determined by supplying a valve in order to deliver a measured amount. The compositions according to the invention can be reduced to powder form with a view to an administration by inhalation. In this case, the compositions according to the invention in powder form can be mixed with a second powder, called base powder, such as a lactose or starch powder, the mixture of the two powders being packed in the form of cartridges or capsules intended to be used in an inhaler or insufflator.

The present invention also relates to pulmonary administration. The pharmaceutical compositions can be delivered to the lungs of a mammal while breathing in, and passing through the epithelial mucosa of the lung to the bloodstream. A wide range of mechanical devices designed for pulmonary delivery of therapeutic products, comprising, but not limited thereto, nebulizers, metered-dose inhalers and powder inhalers, all known to specialists in the art, are envisaged for use in the present invention.

Nasal delivery of a pharmaceutical composition described herein is also envisaged. Nasal delivery allows the passage of a pharmaceutical composition of the present invention to the bloodstream directly after the administration of the therapeutic product to the nose, without the need to deposit the product in the lungs. The formulations for nasal administration comprise those with dextran or cyclodextrin, as well as bio-adhesive excipients such as, for example, chitosan.

The pharmaceutical compositions can also be formulated in rectal or vaginal compositions such as suppositories or retention douches, for example containing conventional suppository bases such as cocoa butter or other glycerides.

The pharmaceutical compositions can also comprise suitable carriers or excipients that are solid or in gel phase. Examples of such carriers or excipients comprise, without being limited thereto, calcium carbonate, calcium phosphate, various sugars, starches, cellulose derivatives, gelatine and polymers such as polyethylene glycols.

For example, appropriate liquid or solid pharmaceutical preparation forms are aqueous or saline solutions for inhalation, are microencapsulated, are notched, are applied to microscopic gold particles, are contained in liposomes, are nebulized, are aerosols, are lozenges for implantation in the skin. The pharmaceutical compositions also comprise granules, powders, tablets, coated tablets, (micro)capsules, suppositories, syrups, emulsions, suspensions, creams, transdermal or cutaneous patches, drops or delayed-release preparations of active and/or auxiliary compositions such as disintegrating agents, binders, coating agents, expanding agents, lubricants, flavourings, sweeteners or solubilizers that are habitually used as described above. The pharmaceutical compositions are suitable for use in various systems for administering medicaments.

Another subject of the invention relates to a method for preventive treatment of inflammatory diseases, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

Within the meaning of the present invention, the term “prevention” or “prophylaxis” or “preventive treatment” or “prophylactic treatment” comprises a treatment leading to the prevention of a disease as well as a treatment reducing and/or retarding the incidence of a disease or the risk of occurrence of the disease.

According to the present invention, the inhibitor of the ROCK-PDK1 protein kinase complex as defined above is particularly efficacious for preventing the appearance of inflammatory diseases, by inhibiting the production of inflammatory cytokines, thus inducing a protective effect against inflammatory diseases. Advantageously, the inventors have shown that the compounds of formulae (IV) and (VII) are capable of inhibiting the production of inflammatory cytokines by the immune cells (monocytes) of healthy donors in response to the activation of the inflammasome by uric acid crystals (mimicking “gout”).

In a particular embodiment of the invention, the present invention relates to a method for preventive treatment of inflammatory diseases, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above and a second active principle as defined above.

Advantageously, the inflammatory disease can be selected from: the amyloid neurodegenerative diseases, such as Alzheimer's disease, the prions, Parkinson's disease, amyotrophic lateral sclerosis; inflammatory diseases of the intestine, such as chronic inflammatory bowel disease (CIBD), irritable bowel syndrome (IBS), Crohn's disease, ulcerative colitis, hemorrhagic rectal ulcer, lesions associated with Behget's disease, pouchitis, ulcerative colitis, ileitis and enteritis; the acute inflammatory diseases such as gout and septic shock, chronic inflammatory lumbar pain, the inflammatory diseases of the skin or dermatitis, such as psoriasis, atopic dermatitis, rosacea, acne erythematosa, acne, common warts, bullous skin diseases, contact eczema, skin cancers, rubor, erythemas, telangiectasias, the inflammations of the skin linked to exposure to UV, such as photo-irritation, photo-sensitization, photo-ageing, photo-carcinogenesis, lymphatic venous insufficiencies or heavy leg syndrome; arthritis, such as osteoarthritis, rheumatoid arthritis, juvenile idiopathic arthritis and psoriatic arthritis; chronic obstructive pulmonary disease (COPD); coeliac disease; chronic pancreatitis; Hashimoto's thyroiditis; primary biliary cirrhosis; sclerosing cholangitis; autoimmune hepatitis; vasculitides, such as systemic vasculitides associated with ANCA (anti-neutrophil cytoplasmic antibodies); spondyloarthropathies; chronic atrophying polychondritis; diabetes; scleroderma and cancer, the list being non-limitative.

In a particularly advantageous embodiment, the present invention relates to a method for preventive treatment of amyloid neurodegenerative diseases, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

Advantageously, the amyloid neurodegenerative diseases can be selected from: Alzheimer's disease, the prions, Parkinson's disease, amyotrophic lateral sclerosis, the list being non-limitative.

In a particularly advantageous embodiment, the present invention relates to a method for preventive treatment of acute inflammatory diseases, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

Advantageously, the acute inflammatory diseases can be selected from: gout and septic shock, the list being non-limitative.

In a particularly advantageous embodiment, the present invention relates to the use of a pharmaceutical composition comprising a therapeutically efficacious amount of an inhibitor of the ROCK-PDK1 protein kinase complex as defined above as active principle in the production of a medicament intended for the prevention of inflammatory diseases.

In a particularly advantageous embodiment, the present invention relates to the use of a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above in the production of a medicament intended for the prevention of inflammatory diseases.

In a particularly advantageous embodiment, the present invention relates to a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above for use thereof in the prevention of inflammatory diseases.

In a particularly advantageous embodiment, the present invention relates to a pharmaceutical composition comprising a therapeutically efficacious amount of an inhibitor of the ROCK protein kinase as defined above as active principle and at least one pharmaceutically acceptable excipient and/or carrier and/or a diluent and/or a pharmaceutically acceptable vehicle as defined above for use thereof in the prevention of inflammatory diseases.

In a particularly advantageous embodiment, the present invention relates to a pharmaceutical composition comprising a therapeutically efficacious amount of an inhibitor of the PDK1 protein kinase as defined above as active principle and at least one pharmaceutically acceptable excipient and/or carrier and/or a diluent and/or a pharmaceutically acceptable vehicle as defined above for use thereof in the prevention of inflammatory diseases.

In a particularly advantageous embodiment, the present invention relates to a pharmaceutical composition comprising a therapeutically efficacious amount of a combination comprising an inhibitor of the ROCK protein kinase and an inhibitor of the PDK1 protein kinase as defined above as active principle and at least one pharmaceutically acceptable excipient and/or carrier and/or a diluent and/or a pharmaceutically acceptable vehicle as defined above for use thereof in the prevention of inflammatory diseases.

In a particularly advantageous embodiment, the present invention relates to a pharmaceutical composition comprising a therapeutically efficacious amount of an agent dissociating the ROCK-PDK1 protein kinase complex as defined above as active principle and at least one pharmaceutically acceptable excipient and/or carrier and/or a diluent and/or a pharmaceutically acceptable vehicle as defined above for use thereof in the prevention of inflammatory diseases.

Another subject of the invention relates to a method for preventive treatment of autoimmune diseases, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

According to the present invention, the inhibitor of the ROCK-PDK1 protein kinase complex as defined above is particularly efficacious for preventing the appearance of autoimmune diseases, by inhibiting the production of inflammatory cytokines, thus inducing a protective effect against autoimmune diseases.

In a particular embodiment of the invention, the present invention relates to a method for preventive treatment of autoimmune diseases, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above and a second active principle as defined above.

Advantageously, the autoimmune disease can be selected from: disseminated lupus erythematosus, systemic lupus erythematosus, multiple sclerosis, rheumatoid polyarthritis, insulin-dependent diabetes, thrombotic thrombocytopenic purpura (TTP), graft or organ rejection, graft versus host disease, the different types of sclerosis, primary Sjogren's syndrome (or Gougerot-Sjogren syndrome), the autoimmune polyneuropathies such as multiple sclerosis, type I diabetes, autoimmune hepatitises, ankylosing spondylitis, Reiter's syndrome, gout arthritis, chronic Hashimoto's thyroiditis (hypothyroidism), Addison's disease, autoimmune hepatitises, Basedow's disease (hyperthyroidism), the autoimmune cytopenias and other hematological complications of adult and child, such as acute or chronic autoimmune thrombocytopenias, autoimmune hemolytic anaemias, hemolytic disease of the newborn (HDN), cold agglutinin disease, thrombotic thrombocytopenic purpura and autoimmune acquired hemophilia; Goodpasture syndrome, the extra-membranous nephropathies, the autoimmune bullous skin disorders, refractory myasthenia gravis, mixed cryoglobulinemias, inflammatory myositis, dermatomyositis and childhood systemic autoimmune disorders including antiphospholipid syndrome, conjunctive tissue disease, the different types of sclerosis, autoimmune pulmonary inflammation, Guillain-Barre syndrome, chronic inflammatory demyelinating polyradiculoneuropathy (CIDP), autoimmune thyroiditis, diabetes mellitus, myasthenia gravis, autoimmune inflammatory disease of the eye, neuromyelitis optica (Devic's disease), the sclerodermas, pemphigus, insulin-resistant diabetes, polymyositis, Biermer's anaemia, glomerulonephritis, Wegener's disease, Horton's disease, periarthritis nodosa and Churg Strauss syndrome, Still's disease, atrophic polychondritis, Behget's disease, monoclonal gammopathy, Wegener's granulomatosis, lupus, psoriatic rheumatism, sarcoidosis, collagenous colitis, dermatitis herpetiformis, familial Mediterranean fever, IgA deposition glomerulonephritis, Lambert-Eaton myasthenic syndrome, ophthalmia sympathica, Fiessinger-Leroy-Reiter syndrome and uveomeningoencephalitic syndrome, the list being non-limitative.

In a particularly advantageous embodiment, the present invention relates to a method for preventive treatment of rheumatoid polyarthritis, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

In a particularly advantageous embodiment, the present invention relates to a method for preventive treatment of disseminated lupus erythematosus, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

In a particularly advantageous embodiment, the present invention relates to the use of a pharmaceutical composition comprising a therapeutically efficacious amount of an inhibitor of the ROCK-PDK1 protein kinase complex as defined above as active principle in the production of a medicament intended for the prevention of autoimmune diseases.

In a particularly advantageous embodiment, the present invention relates to the use of a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above in the production of a medicament intended for the prevention of autoimmune diseases.

In a particularly advantageous embodiment, the present invention relates to a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above for use thereof in the prevention of autoimmune diseases.

In a particularly advantageous embodiment, the present invention relates to a pharmaceutical composition comprising a therapeutically efficacious amount of an inhibitor of the ROCK protein kinase as defined above as active principle and at least one pharmaceutically acceptable excipient and/or carrier and/or a diluent and/or a pharmaceutically acceptable vehicle as defined above for use thereof in the prevention of autoimmune diseases.

In a particularly advantageous embodiment, the present invention relates to a pharmaceutical composition comprising a therapeutically efficacious amount of an inhibitor of the PDK1 protein kinase as defined above as active principle and at least one pharmaceutically acceptable excipient and/or carrier and/or a diluent and/or a pharmaceutically acceptable vehicle as defined above for use thereof in the prevention of autoimmune diseases.

In a particularly advantageous embodiment, the present invention relates to a pharmaceutical composition comprising a therapeutically efficacious amount of a combination comprising an inhibitor of the ROCK protein kinase and an inhibitor of the PDK1 protein kinase as defined above as active principle and at least one pharmaceutically acceptable excipient and/or carrier and/or a diluent and/or a pharmaceutically acceptable vehicle as defined above for use thereof in the prevention of autoimmune diseases.

In a particularly advantageous embodiment, the present invention relates to a pharmaceutical composition comprising a therapeutically efficacious amount of an agent dissociating the ROCK-PDK1 protein kinase complex as defined above as active principle and at least one pharmaceutically acceptable excipient and/or carrier and/or a diluent and/or a pharmaceutically acceptable vehicle as defined above for use thereof in the prevention of autoimmune diseases.

Another subject of the invention relates to a method for preventive treatment of viral and/or bacterial infections, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

According to the present invention, the inhibitor of the ROCK-PDK1 protein kinase complex is particularly efficacious for preventing the appearance, the development, the aggravation, the persistence of viral and/or bacterial infections, by inhibiting the production of inflammatory cytokines, thus inducing a protective effect against viral and/or bacterial infections. Advantageously, the inventors have shown that the compounds of formulae (IV) and (VII) are capable of inhibiting the production of inflammatory cytokines by the immune cells (PBMCs and purified monocytes) of healthy donors in response to the stimulation of the Toll-like receptors 2/4 by bacterial agents (respectively by lipoteichoic acid/LPS). Advantageously, the inventors have shown that the compounds of formulae (IV) and (VII) are capable of inhibiting the production of inflammatory cytokines by the immune cells (PBMCs and purified monocytes) of healthy donors in response to the stimulation of the Toll-like receptor ⅞ receptors (R848). It has also been demonstrated that the compound of formula (IV) is capable of inhibiting the production of inflammatory cytokines and of the interferons in response to the human immunodeficiency virus (HIV) on purified plasmacytoid dendritic cells and on peripheral blood mononuclear cells (PBMCs).

In a particular embodiment of the invention, the present invention relates to a method for preventive treatment of viral and/or bacterial infections, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above and a second active principle as defined above.

Advantageously, the viral infections can be selected from the infections due to the influenza virus, the human immunodeficiency virus (HIV), the herpes virus or herpes simplex virus (HSV), the coronavirus, COVID-19, the encephalomyocarditis virus, the arboviruses, such as chikungunya, o'nyong'nyong, Ross River, Sindbis, Mayaro viruses, the yellow fever virus, dengue fever virus, Japanese encephalitis virus, the West Nile virus, the temperate Eurasian tick-borne encephalitis viruses, the Kyasanur Forest disease viruses, the Omsk hemorrhagic fever virus, the Bunyavirales viruses, the Bunyamwera virus, the Rift Valley fever virus, the Crimean-Congo hemorrhagic fever virus, the hepatitis A, B and C virus and the influenza virus, the list being non-limitative.

Advantageously, the bacterial infections can be selected from: the infections due to gram-positive bacteria, such as the bacteria of the genus Staphylococcus in particular Staphylococcus aureus, and the bacteria of the genus Enterococcus, in particular Enterococcus faecalis; the infections due to gram-negative bacteria, such as the bacteria of the genus Escherichia, in particular Escherichia coli, the bacteria of the genus Pseudomonas, in particular Pseudomonas aeruginosa, and the bacteria of the genus Acinetobacter, in particular Acinetobacterbaumannii, the list being non-limitative.

In a particularly advantageous embodiment, the present invention relates to a method for preventive treatment of the coronavirus infections, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

In a particularly advantageous embodiment, the present invention relates to a method for preventive treatment of infections due to the influenza virus, comprising the administration to the patient of an inhibitor of the ROCK-PDK1 protein kinase complex or a pharmaceutical composition comprising a therapeutically efficacious amount of an inhibitor of the ROCK-PDK1 protein kinase complex as active principle and at least one pharmaceutically acceptable excipient and/or carrier and/or a diluent and/or a pharmaceutically acceptable vehicle as defined above.

In a particularly advantageous embodiment, the present invention relates to a method for preventive treatment of the human immunodeficiency virus (HIV) infections, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

In a particularly advantageous embodiment, the present invention relates to the use of a pharmaceutical composition comprising a therapeutically efficacious amount of an inhibitor of the ROCK-PDK1 protein kinase complex as defined above as active principle in the production of a medicament intended for the prevention of viral and/or bacterial infections.

In a particularly advantageous embodiment, the present invention relates to the use of a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above in the production of a medicament intended for the prevention of viral and/or bacterial infections.

In a particularly advantageous embodiment, the present invention relates to a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above for use thereof in the prevention of viral and/or bacterial infections.

In a particularly advantageous embodiment, the present invention relates to a pharmaceutical composition comprising a therapeutically efficacious amount of an inhibitor of the ROCK protein kinase as defined above as active principle and at least one pharmaceutically acceptable excipient and/or carrier and/or a diluent and/or a pharmaceutically acceptable vehicle as defined above for use thereof in the prevention of viral and/or bacterial infections.

In a particularly advantageous embodiment, the present invention relates to a pharmaceutical composition comprising a therapeutically efficacious amount of an inhibitor of the PDK1 protein kinase as defined above as active principle and at least one pharmaceutically acceptable excipient and/or carrier and/or a diluent and/or a pharmaceutically acceptable vehicle as defined above for use thereof in the prevention of viral and/or bacterial infections.

In a particularly advantageous embodiment, the present invention relates to a pharmaceutical composition comprising a therapeutically efficacious amount of a combination comprising an inhibitor of the ROCK protein kinase and an inhibitor of the PDK1 protein kinase as defined above as active principle and at least one pharmaceutically acceptable excipient and/or carrier and/or a diluent and/or a pharmaceutically acceptable vehicle as defined above for use thereof in the prevention of viral and/or bacterial infections.

In a particularly advantageous embodiment, the present invention relates to a pharmaceutical composition comprising a therapeutically efficacious amount of an agent dissociating the ROCK-PDK1 protein kinase complex as defined above as active principle and at least one pharmaceutically acceptable excipient and/or carrier and/or a diluent and/or a pharmaceutically acceptable vehicle as defined above for use thereof in the prevention of viral and/or bacterial infections.

Another subject of the invention relates to a method for curative treatment of inflammatory diseases in patients needing an administration, comprising the administration to said patients of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

Within the meaning of the present invention, the term “treatment” or “curative treatment” is defined as a treatment leading to recovery or a treatment that improves, ameliorates and/or eliminates, reduces and/or stabilizes the symptoms of a disease or the suffering that it causes.

Advantageously, the inventors have shown that inhibition of the PDK1 protein kinase by the inhibitor of formula (IV) makes it possible to drastically reduce the spontaneous production of inflammatory cytokines by the monocytes originating from patients suffering from juvenile idiopathic arthritis.

Advantageously, the inventors have shown that inhibition of the PDK1 protein kinase by the inhibitor of formula (IV) administered by the oral route at a dose of 10 mg/kg of bodyweight in mouse models for rheumatoid polyarthritis blocked the progression of this pathology by attenuating the production of inflammatory cytokines.

Another subject of the invention relates to a method for curative treatment of inflammatory diseases in patients needing an administration, comprising the administration to the patients of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above and a second active principle as defined above.

Advantageously, the inflammatory disease can be selected from: the amyloid neurodegenerative diseases, such as Alzheimer's disease, the prions, Parkinson's disease, amyotrophic lateral sclerosis; inflammatory diseases of the intestine, such as chronic inflammatory bowel disease (CIBD), irritable bowel syndrome (IBS), Crohn's disease, ulcerative colitis, hemorrhagic rectal ulcer, lesions associated with Behget's disease, pouchitis, ulcerative colitis, ileitis and enteritis; the acute inflammatory diseases such as gout and septic shock, chronic inflammatory lumbar pain, the inflammatory diseases of the skin or dermatitis, such as psoriasis, atopic dermatitis, rosacea, acne erythematosa, acne, common warts, bullous skin diseases, contact eczema, skin cancers, rubor, erythemas, telangiectasias, the inflammations of the skin linked to exposure to UV, such as photo-irritation, photo-sensitization, photo-ageing, photo-carcinogenesis, lymphatic venous insufficiencies or heavy leg syndrome; arthritis, such as osteoarthritis, rheumatoid arthritis, juvenile idiopathic arthritis and psoriatic arthritis; chronic obstructive pulmonary disease (COPD); coeliac disease; chronic pancreatitis; Hashimoto's thyroiditis; primary biliary cirrhosis; sclerosing cholangitis; autoimmune hepatitis; vasculitides, such as systemic vasculitides associated with ANCA (anti-neutrophil cytoplasmic antibodies); spondyloarthropathies; chronic atrophying polychondritis; diabetes; scleroderma and cancer, the list being non-limitative.

In a particularly advantageous embodiment, the present invention relates to a method for curative treatment of the amyloid neurodegenerative diseases, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

Advantageously, the amyloid neurodegenerative diseases can be selected from: Alzheimer's disease, the prions, Parkinson's disease, amyotrophic lateral sclerosis, the list being non-limitative.

In a particularly advantageous embodiment, the present invention relates to a method for curative treatment of acute inflammatory diseases, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

Advantageously, the inflammatory diseases can be selected from: gout and septic shock, the list being non-limitative.

In a particularly advantageous embodiment, the present invention relates to the use of a pharmaceutical composition comprising a therapeutically efficacious amount of an inhibitor of the ROCK-PDK1 protein kinase complex as defined above as active principle in the production of a medicament intended for the treatment of inflammatory diseases.

In a particularly advantageous embodiment, the present invention relates to the use of a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above in the production of a medicament intended for the treatment of inflammatory diseases.

In a particularly advantageous embodiment, the present invention relates to a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above for use thereof in the treatment of inflammatory diseases.

In a particularly advantageous embodiment, the present invention relates to a pharmaceutical composition comprising a therapeutically efficacious amount of an inhibitor of the ROCK protein kinase as defined above as active principle and at least one pharmaceutically acceptable excipient and/or carrier and/or a diluent and/or a pharmaceutically acceptable vehicle as defined above for use thereof in the treatment of inflammatory diseases.

In a particularly advantageous embodiment, the present invention relates to a pharmaceutical composition comprising a therapeutically efficacious amount of an inhibitor of the PDK1 protein kinase as defined above as active principle and at least one pharmaceutically acceptable excipient and/or carrier and/or a diluent and/or a pharmaceutically acceptable vehicle as defined above for use thereof in the treatment of inflammatory diseases.

In a particularly advantageous embodiment, the present invention relates to a pharmaceutical composition comprising a therapeutically efficacious amount of a combination comprising an inhibitor of the ROCK protein kinase and an inhibitor of the PDK1 protein kinase as defined above as active principle and at least one pharmaceutically acceptable excipient and/or carrier and/or a diluent and/or a pharmaceutically acceptable vehicle as defined above for use thereof in the treatment of inflammatory diseases.

In a particularly advantageous embodiment, the present invention relates to a pharmaceutical composition comprising a therapeutically efficacious amount of an agent dissociating the ROCK-PDK1 protein kinase complex as defined above as active principle and at least one pharmaceutically acceptable excipient and/or carrier and/or a diluent and/or a pharmaceutically acceptable vehicle as defined above for use thereof in the treatment of inflammatory diseases.

Another subject of the invention relates to a method for curative treatment of autoimmune diseases, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

According to the present invention, the inhibitor of the ROCK-PDK1 protein kinase complex is particularly efficacious for treating autoimmune diseases, by inhibiting the production of inflammatory cytokines, thus inducing a curative effect against autoimmune diseases.

In a particular embodiment of the invention, the present invention relates to a method for curative treatment of autoimmune diseases, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above and a second active principle as defined above.

Advantageously, the autoimmune disease can be selected from: disseminated lupus erythematosus, systemic lupus erythematosus, multiple sclerosis, rheumatoid polyarthritis, insulin-dependent diabetes, thrombotic thrombocytopenic purpura (TTP), graft or organ rejection, graft versus host disease, the different types of sclerosis, primary Sjogren's syndrome (or Gougerot-Sjogren syndrome), the autoimmune polyneuropathies such as multiple sclerosis, type I diabetes, autoimmune hepatitises, ankylosing spondylitis, Reiter's syndrome, gout arthritis, chronic Hashimoto's thyroiditis (hypothyroidism), Addison's disease, autoimmune hepatitises, Basedow's disease (hyperthyroidism), the autoimmune cytopenias and other hematological complications of adult and child, such as acute or chronic autoimmune thrombocytopenias, autoimmune hemolytic anaemias, hemolytic disease of the newborn (HDN), cold agglutinin disease, thrombotic thrombocytopenic purpura and autoimmune acquired hemophilia; Goodpasture syndrome, the extra-membranous nephropathies, the autoimmune bullous skin disorders, refractory myasthenia gravis, mixed cryoglobulinemias, inflammatory myositis, dermatomyositis and childhood systemic autoimmune disorders including antiphospholipid syndrome, conjunctive tissue disease, the different types of sclerosis, autoimmune pulmonary inflammation, Guillain-Barre syndrome, chronic inflammatory demyelinating polyradiculoneuropathy (CIDP), autoimmune thyroiditis, diabetes mellitus, myasthenia gravis, autoimmune inflammatory disease of the eye, neuromyelitis optica (Devic's disease), the sclerodermas, pemphigus, insulin-resistant diabetes, polymyositis, Biermer's anaemia, glomerulonephritis, Wegener's disease, Horton's disease, periarthritis nodosa and Churg Strauss syndrome, Still's disease, atrophic polychondritis, Behget's disease, monoclonal gammopathy, Wegener's granulomatosis, lupus, psoriatic rheumatism, sarcoidosis, collagenous colitis, dermatitis herpetiformis, familial Mediterranean fever, IgA deposition glomerulonephritis, Lambert-Eaton myasthenic syndrome, ophthalmia sympathica, Fiessinger-Leroy-Reiter syndrome and uveomeningoencephalitic syndrome, the list being non-limitative.

In a particularly advantageous embodiment, the present invention relates to a method for curative treatment of rheumatoid polyarthritis, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

In a particularly advantageous embodiment, the present invention relates to a method for curative treatment of disseminated lupus erythematosus, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

In a particularly advantageous embodiment, the present invention relates to the use of a pharmaceutical composition comprising a therapeutically efficacious amount of an inhibitor of the ROCK-PDK1 protein kinase complex as defined above as active principle in the production of a medicament intended for the treatment of autoimmune diseases.

In a particularly advantageous embodiment, the present invention relates to the use of a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above in the production of a medicament intended for the treatment of autoimmune diseases.

In a particularly advantageous embodiment, the present invention relates to a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above for use thereof in the treatment of autoimmune diseases.

In a particularly advantageous embodiment, the present invention relates to a pharmaceutical composition comprising a therapeutically efficacious amount of an inhibitor of the ROCK protein kinase as defined above as active principle and at least one pharmaceutically acceptable excipient and/or carrier and/or a diluent and/or a pharmaceutically acceptable vehicle as defined above for use thereof in the treatment of autoimmune diseases.

In a particularly advantageous embodiment, the present invention relates to a pharmaceutical composition comprising a therapeutically efficacious amount of an inhibitor of the PDK1 protein kinase as defined above as active principle and at least one pharmaceutically acceptable excipient and/or carrier and/or a diluent and/or a pharmaceutically acceptable vehicle as defined above for use thereof in the treatment of autoimmune diseases.

In a particularly advantageous embodiment, the present invention relates to a pharmaceutical composition comprising a therapeutically efficacious amount of a combination comprising an inhibitor of the ROCK protein kinase and an inhibitor of the PDK1 protein kinase as defined above as active principle and at least one pharmaceutically acceptable excipient and/or carrier and/or a diluent and/or a pharmaceutically acceptable vehicle as defined above for use thereof in the treatment of autoimmune diseases.

In a particularly advantageous embodiment, the present invention relates to a pharmaceutical composition comprising a therapeutically efficacious amount of an agent dissociating the ROCK-PDK1 protein kinase complex as defined above as active principle and at least one pharmaceutically acceptable excipient and/or carrier and/or a diluent and/or a pharmaceutically acceptable vehicle as defined above for use thereof in the treatment of autoimmune diseases.

Another subject of the invention relates to a method for curative treatment of viral and/or bacterial infections, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

According to the present invention, the inhibitor of the ROCK-PDK1 protein kinase complex as defined above is particularly efficacious for treating autoimmune diseases, by inhibiting the production of inflammatory cytokines, thus inducing a curative effect against viral and/or bacterial infections.

In a particular embodiment of the invention, the present invention relates to a method for curative treatment of viral and/or bacterial infections, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above and a second active principle as defined above.

Advantageously, the viral infections can be selected from the infections due to the influenza virus, the human immunodeficiency virus (HIV), the herpes virus or herpes simplex virus (HSV), the coronavirus, COVID-19, the encephalomyocarditis virus, the arboviruses, such as chikungunya, o'nyong'nyong, Ross River, Sindbis, Mayaro viruses, the yellow fever virus, dengue fever virus, Japanese encephalitis virus, the West Nile virus, the temperate Eurasian tick-borne encephalitis viruses, the Kyasanur Forest disease viruses, the Omsk hemorrhagic fever virus, the Bunyavirales viruses, the Bunyamwera virus, the Rift Valley fever virus, the Crimean-Congo hemorrhagic fever virus, the hepatitis A, B and C virus and the influenza virus, the list being non-limitative.

Advantageously, the bacterial infections can be selected from: the infections due to gram-positive bacteria, such as the bacteria of the genus Staphylococcus in particular Staphylococcus aureus, and the bacteria of the genus Enterococcus, in particular Enterococcus faecalis; the infections due to gram-negative bacteria, such as the bacteria of the genus Escherichia, in particular Escherichia coli, the bacteria of the genus Pseudomonas, in particular Pseudomonas aeruginosa, and the bacteria of the genus Acinetobacter, in particular Acinetobacterbaumannii, the list being non-limitative.

In a particularly advantageous embodiment, the present invention relates to a method for curative treatment of the coronavirus infections, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

In a particularly advantageous embodiment, the present invention relates to a method for curative treatment of the infections due to the influenza virus, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

In a particularly advantageous embodiment, the present invention relates to a method for curative treatment of the human immunodeficiency virus (HIV) infections, comprising the administration to the patient of an inhibitor of the ROCK-PDK1 protein kinase complex as defined above or a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above.

In a particularly advantageous embodiment, the present invention relates to the use of a pharmaceutical composition comprising a therapeutically efficacious amount of an inhibitor of the ROCK-PDK1 protein kinase complex as defined above as active principle in the production of a medicament intended for the treatment of viral and/or bacterial infections.

In a particularly advantageous embodiment, the present invention relates to the use of a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above in the production of a medicament intended for the treatment of viral and/or bacterial infections.

In a particularly advantageous embodiment, the present invention relates to a pharmaceutical composition comprising a therapeutically efficacious amount of an inhibitor of the ROCK-PDK1 protein kinase complex as defined above as active principle and at least one pharmaceutically acceptable excipient and/or carrier and/or a diluent and/or a pharmaceutically acceptable vehicle as defined above for use thereof in the treatment of viral and/or bacterial infections.

In a particularly advantageous embodiment, the present invention relates to a pharmaceutical composition comprising a therapeutically efficacious amount of an inhibitor of the ROCK protein kinase as defined above as active principle and at least one pharmaceutically acceptable excipient and/or carrier and/or a diluent and/or a pharmaceutically acceptable vehicle as defined above for use thereof in the treatment of viral and/or bacterial infections.

In a particularly advantageous embodiment, the present invention relates to a pharmaceutical composition comprising a therapeutically efficacious amount of an inhibitor of the PDK1 protein kinase as defined above as active principle and at least one pharmaceutically acceptable excipient and/or carrier and/or a diluent and/or a pharmaceutically acceptable vehicle as defined above for use thereof in the treatment of viral and/or bacterial infections.

In a particularly advantageous embodiment, the present invention relates to a pharmaceutical composition comprising a therapeutically efficacious amount of a combination comprising an inhibitor of the ROCK protein kinase and an inhibitor of the PDK1 protein kinase as defined above as active principle and at least one pharmaceutically acceptable excipient and/or carrier and/or a diluent and/or a pharmaceutically acceptable vehicle as defined above for use thereof in the treatment of viral and/or bacterial infections.

In a particularly advantageous embodiment, the present invention relates to a pharmaceutical composition comprising a therapeutically efficacious amount of an agent dissociating the ROCK-PDK1 protein kinase complex as defined above as active principle and at least one pharmaceutically acceptable excipient and/or carrier and/or a diluent and/or a pharmaceutically acceptable vehicle as defined above for use thereof in the treatment of viral and/or bacterial infections.

### FIGURES

FIG. 1: FIG. 1 shows the blocking by an inhibitor of the ROCK protein kinase of formula (VII) (also called Y-27632) of the intracellular production of TNFα on monocytes of healthy donors in response to a stimulation of the Toll-like receptor TLR7/8 receptors (Resiquimod-R848), results obtained by flow cytometry.

FIG. 2: FIG. 2 shows the blocking by an inhibitor of the PDK1 protein kinase of formula (IV) (also called BX912) of the intracellular production of TNFα on monocytes of healthy donors in response to a stimulation of the Toll-like receptor TLR7/8 receptors (Resiquimod-R848), results obtained by flow cytometry.

FIG. 3: FIG. 3 shows the blocking by an inhibitor of the PDK1 protein kinase of formula (IV) (also called BX912) of the secretion of inflammatory cytokines and interferons on peripheral blood mononuclear cells (PBMCs) of healthy donors in response to a stimulation of the Toll-like receptor TLR7/8 receptors. Results obtained by LegendPlex.

FIG. 4: FIG. 4 shows the absence of toxicity of the inhibitor of the PDK1 protein kinase of formula (IV) (also called BX912) on peripheral blood mononuclear cells (PBMCs) of healthy donors.

FIG. 5: FIG. 5 shows the blocking of the intracellular production of TNFα on monocytes of healthy donors by an inhibitor of the PDK1 protein kinase of formula (IV) (also called BX912) in response to a stimulation of the Toll-like receptor 4 receptors (lipopolysaccharides-LPS), results obtained by flow cytometry.

FIG. 6: FIG. 6 shows the blocking of the intracellular production of TNFα on monocytes of healthy donors by an inhibitor of the PDK1 protein kinase of formula (IV) (also called BX912) in response to a stimulation of the Toll-like receptor 2 receptors (lipoteichoic acid-LTA), results obtained by flow cytometry.

FIG. 7: FIG. 7 shows the blocking by an inhibitor of the PDK1 protein kinase of formula (IV) (also called BX912) of the secretion of inflammatory cytokines and interferons on peripheral blood mononuclear cells (PBMCs) of healthy donors in response to a stimulation of the Toll-like receptor TLR4 receptors. Results obtained by LegendPlex.

FIG. 8: FIG. 8 shows the absence of toxicity of the inhibitor of the PDK1 protein kinase of formula (IV) (also called BX912) on peripheral blood mononuclear cells (PBMCs) of healthy donors.

FIG. 9: FIG. 9 shows the blocking of the intracellular production of IL13 on monocytes of healthy donors by an inhibitor of the PDK1 protein kinase of formula (IV) (also called BX912) in response to an activation of the inflammasome by uric acid crystals (MSU) (“gout” model), results obtained by flow cytometry.

FIG. 10: FIG. 10 shows the blocking of the intracellular production of TNFα on monocytes of healthy donors by an inhibitor of the PDK1 protein kinase of formula (IV) (also called BX912) in response to an activation of the inflammasome by uric acid crystals (MSU) (“gout” model), results obtained by flow cytometry.

FIG. 11: FIG. 11 shows the blocking of the intracellular production of IFNα on pDCs of healthy donors by an inhibitor of the PDK1 protein kinase of formula (IV) (also called BX912) in response to an infection with HIV, results obtained by flow cytometry.

FIG. 12: FIG. 12 shows the blocking of the intracellular production of TNFα on pDCs of healthy donors by an inhibitor of the PDK1 protein kinase of formula (IV) (also called BX912) in response to an infection with HIV, results obtained by flow cytometry.

FIG. 13: FIG. 13 shows the blocking by an inhibitor of the PDK1 protein kinase of formula (IV) (also called BX912) of the secretion of inflammatory cytokines and interferons on peripheral blood mononuclear cells (PBMCs) of healthy donors in response to an infection with HIV. Results obtained by LegendPlex.

FIG. 14: FIG. 14 shows the absence of toxicity of the inhibitor of the PDK1 protein kinase of formula (IV) (also called BX912) on peripheral blood mononuclear cells (PBMCs) of healthy donors.

FIG. 15A: FIG. 15A shows the blocking by the inhibitor of formula (IV) (also called BX912) of the secretion of interferons on peripheral blood mononuclear cells (PBMCs) of healthy donors, in a preventive manner, 1 h before the stimulation by Toll-like TLR ⅞ receptors. Results obtained with the STING-37 reporter cell line.

FIG. 15B: FIG. 15B shows the blocking by the inhibitor of formula (IV) (also called BX912) of the secretion of interferons on peripheral blood mononuclear cells (PBMCs) of healthy donors, during the stimulation by Toll-like TLR ⅞ receptors. Results obtained with the STING-37 reporter cell line.

FIG. 15C: FIG. 15C shows the blocking by the inhibitor of formula (IV) (also called BX912) of the secretion of interferons on peripheral blood mononuclear cells (PBMCs) of healthy donors, in a curative manner, 1 h after the stimulation by Toll-like TLR ⅞ receptors. Results obtained with the STING-37 reporter cell line.

FIG. 16: FIG. 16 shows the blocking by the inhibitor of formula (IV) (also called BX912) of the spontaneous production of TNFα on monocytes of patients suffering from juvenile idiopathic arthritis.

Results obtained by SIMOA assay (digital ELISA).

FIG. 17: FIG. 17 represents the effect of the administration of vehicle, of prednisolone and of BX912 on the weight of mice suffering from human rheumatoid polyarthritis.

FIG. 18: FIG. 18 represents the effect of the administration of vehicle, of prednisolone and of BX912 on the progression of the rheumatoid polyarthritis.

FIG. 19: FIG. 19 represents the effect of the administration of vehicle, of prednisolone and of BX912 on the terminal plasma TNFα content.

FIG. 20: FIG. 20 represents the effect of the administration of vehicle, of prednisolone and of BX912 on the terminal plasma IFNγ content.

FIG. 21: FIG. 21 represents the effect of the administration of vehicle, of prednisolone and of BX912 on the terminal plasma IL-6 content.

FIG. 22: FIG. 22 represents the effect of the administration of vehicle, of prednisolone and of BX912 on the terminal plasma IL-1β content.

FIG. 23: FIG. 23 represents the effect of the administration of vehicle, of prednisolone and of BX912 on the histological parameters of rheumatoid polyarthritis (inflammation, pannus, damage to the cartilage, bone resorption/damage).

FIG. 24: FIG. 24 shows the inhibition of the activation of T lymphocytes by the inhibitor of the PDK1 protein kinase of formula (IV) (also called BX912). Purified T lymphocytes from healthy donors were treated with the inhibitor of the PDK1 protein kinase of formula (IV) for 1 h, at 1 and 5 μM, then activated with CD3/CD28 beads for 3 days. Graphical representation of the percentage of T lymphocytes expressing CD69 (A) and graphical representation of the MFI (B) (results obtained by flow cytometry).

FIG. 25: FIG. 25 shows the inhibition of the activation of B lymphocytes by the inhibitor of the PDK1 protein kinase of formula (IV) (also called BX912). Purified B lymphocytes from healthy donors were treated with the inhibitor of the PDK1 protein kinase of formula (IV) for 1 h, at 1 μM, then activated with CpG-A (1 μg/ml) for 24 h. Graphical representation of the percentage of T lymphocytes expressing CD69 (A) and graphical representation of the MFI (B) (results obtained by flow cytometry).

FIG. 26: FIG. 26 shows the immunosuppressive effect of the inhibitor of the PDK1 protein kinase of formula (IV) (also called BX912) in immune and non-immune cells. (A) Human PBMCs of healthy donors were treated with the PDK1 inhibitor (compound BX912) for 1 h, following the doses indicated, then activated with cGAMP for 24 h. The interferons in the culture supernatants of the PBMCs were assayed with the STING-37 reporter cell line. (B) THP1-Dual and (C) HEK293 were treated with the inhibitor of the PDK1 protein kinase of formula (IV) for 1 h, following the doses indicated, then activated with cGAMP for 24 h. The production of IFN was measured via luciferase.

FIG. 27: FIG. 27 shows the immunosuppressive effect of the inhibitor of the PDK1 protein kinase of formula (IV) (also called BX912) in immune and non-immune cells. (A) THP1-Dual, (B) 1C11 neuroectodermal stem cells and (C) 1C11^{5-HT} serotonergic neurons were treated with the inhibitor of the PDK1 protein kinase of formula (IV) for 1 h, following the doses indicated, then activated with LPS for 1 h. mRNA encoding TNFα, IL6 and MCP1 was quantified by RT-qPCR.

### EXAMPLES

### Example 1: Isolation and Culturing of Blood Leukocytes

The in vitro experiments were carried out using human peripheral blood mononuclear cells (PBMCs) isolated by density gradient centrifugation by means of a medium for separating leukocytes from peripheral blood (STEMCELL Technologies). The blood of healthy donors was obtained from the “Etablissement Frangais du Sang” [French Blood Establishment] (convention #07/CABANEL/106; Paris, France). The studies on material from patients were approved by the research ethics committee (ID-RCB/EUDRACT: 2014-A01017-40 and 2018-A01358-47). The human monocytes were purified by positive selection with human CD14 microbeads (Miltenyi). The plasmacytoid dendritic cells (pDC) were purified by negative selection with the “EasySep Human Plasmacytoid DC” Enrichment Kit (STEMCELL Technologies). The peripheral blood mononuclear cells (PBMCs), monocytes and plasmacytoid dendritic cells (pDC) were cultured in RPMI 1640 (Sigma) containing 10% inactivated foetal bovine serum and 1 mM glutamine (Hyclone, Logan, UT).

Example 2: Preventive Effect of the Inhibition of the PDK1 Protein Kinase by the Inhibitor of Formula (IV) (Also Called BX912) or of the Inhibition of the ROCK Protein Kinase by the Inhibitor of Formula (VII) (Also Called Y-27632) in Response to the Stimulation of the Toll ⅞ Receptors

### Cell Stimulation

The PBMCs were cultured at 2.10⁶ cells/ml. The monocytes were cultured at 1.10⁶ cells/ml. The cells were then incubated with the inhibitor of the PDK1 protein kinase of formula (IV) (also called BX912) or the inhibitor of the ROCK protein kinase of formula (VII) (also called Y27632) at the concentrations indicated for 1 h before stimulation. They were stimulated for 16 h (flow cytometry) or 24 h (LegendPlex) with the TLR7/8 agonist, Resiquimod—R848, at 5 μg/ml.

The cells were recovered for the flow cytometry and the supernatants were collected for the detection of the cytokines. For the intracellular labelling of TNFα and IL-1β, Brefeldin A (BFA) was added to the cells 30 minutes after the stimulation.

### Flow Cytometry

The cells were rinsed in PBS then incubated with a viability dye (Zombie Aqua, Biolegend) for 30 min at ambient temperature. After washing, the cells were resuspended in PBS containing 2% SVF and 2 mM EDTA then labelled with the APC Vio770 anti-CD14 (clone REA599) antibody from Miltenyi Biotec, used at 1/100°. For the intracellular labelling of TNFα and IL-1β, the “Inside Stain” kit (Miltenyi Biotec) was used according to the manufacturer's protocol. The cells were fixed for 20 min at ambient temperature with 250 μL Inside Fix solution then labelled in 100 μL Inside Perm solution containing the PE anti-TNFα (clone cA2, Miltenyi Biotec) and/or APC anti-IL-1β (clone REA1172, Miltenyi Biotec) antibody at 1/50° for 30 min at ambient temperature. Data acquisition was carried out on the Canto II flow cytometer using the Diva software (BD Biosciences, San Jose, CA). The Kaluza software was used to analyze the data.

### Detection of the Cytokines

An assay of the cytokines in the supernatants of the cell cultures was carried out using the “LegendPlex Antivirus Human Panel” or “LegendPlex Human Inflammation Panel” kit (Biolegend, San Diego, USA) according to the manufacturer's instructions.

Test for toxicity of the compounds on the peripheral blood mononuclear cells (PBMCs).

After 24 h of cell culture, the cell viability was determined using the CellTiter-Glo reagent (Promega), which evaluates the amount of ATP by luminescence using the EnSpire.

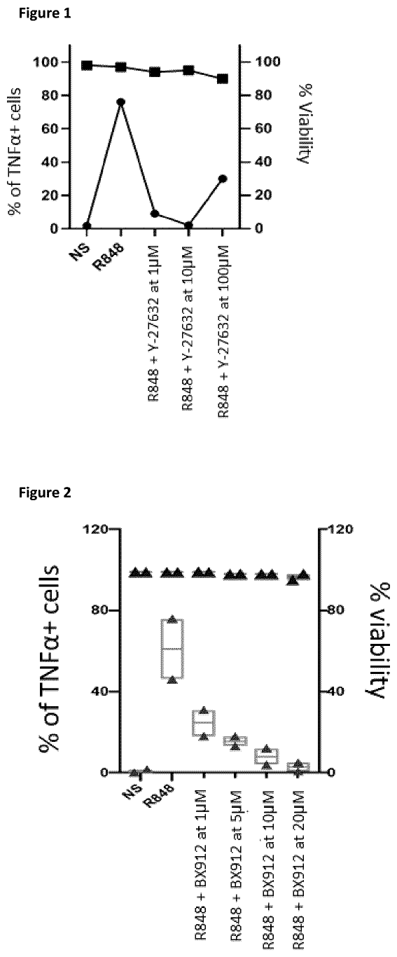

The results are presented in FIGS. 1 to 4.

### Results:

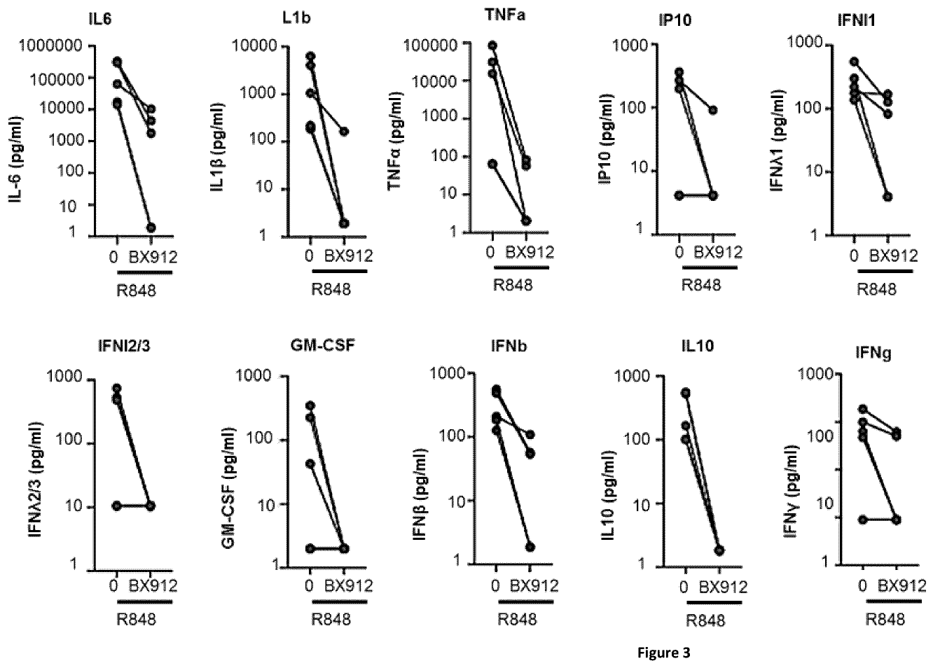

It can be seen that:
inhibition of the ROCK protein kinase by the compound of formula (VII) (also called Y27632) inhibits, according to a dosage effect, the intracellular production of inflammatory cytokines (TNFα) by the monocytes of healthy donors in response to the stimulation of the Toll-like receptor TLR ⅞ receptors (see FIG. 1).
inhibition of the PDK1 protein kinase by the compound of formula (IV) (also called BX912) inhibits, according to a dosage effect, the intracellular production of inflammatory cytokines (TNFα) by monocytes of healthy donors in response to the stimulation of the Toll-like receptor TLR ⅞ receptors (see FIG. 2).
inhibition of the PDK1 protein kinase by the compound of formula (IV) (also called BX912) inhibits the secretion of the inflammatory cytokines (IL-6, IL-1β, TNFα, IP10, GM-CSF, IL-10) and the interferons (IFN11, IFN12/3, IFNβ, IFNg) by the peripheral blood mononuclear cells (PBMCs) of healthy donors in response to the stimulation of the Toll-like receptor TLR ⅞ receptors (see FIG. 3).
the compound of formula (IV) (also called BX912) has no toxicity on the immune cells (PBMCs) of healthy donors in response to the stimulation of the TLR ⅞ receptors (see FIG. 4).

Thus, the compounds of formula (VII) (also called Y27632) and of formula (IV) (also called BX912) have a preventive effect on the production of inflammatory cytokines (TNFα) by the monocytes of healthy donors in response to the stimulation of the Toll-like receptor TLR ⅞ receptors.

In addition, the compound of formula (IV) (also called BX912) is non-toxic. It has a preventive effect on the secretion of the inflammatory cytokines (IL-6, IL-1β, TNFα, IP10, GM-CSF, IL-10) and the interferons (IFN11, IFN12/3, IFNβ, IFNg) by the peripheral blood mononuclear cells (PBMCs) of healthy donors in response to the stimulation of the Toll-like receptor TLR ⅞ receptors.

### Example 3: Preventive Effect of the Inhibition of the PDK1 Protein Kinase by the Inhibitor of Formula (IV) (Also Called BX912) in Response to the Stimulation of the TLR 2/4 Receptors

### Cell Stimulation

The PBMCs were cultured at 2.10⁶ cells/ml. The monocytes were cultured at 1.10⁶ cells/ml. The cells were then incubated with the inhibitor of the PDK1 protein kinase of formula (IV) (also called BX912) or the inhibitor of the ROCK protein kinase of formula (VII) (also called Y27632) at the concentrations indicated for 1 h before stimulation. They were stimulated for 16 h (flow cytometry) or 24 h (LegendPlex), respectively, with the TLR4 agonist, lipopolysaccharide—LPS, at 100 ng/ml and the TLR2 agonist, lipoteichoic acid—LTA, at 1 μg/ml.

The cells were recovered for the flow cytometry and the supernatants were collected for the detection of the cytokines. For the intracellular labelling of TNFα and IL-1β, Brefeldin A (BFA) was added to the cells 30 minutes after the stimulation.

### Flow Cytometry

The cells were rinsed in PBS then incubated with a viability dye (Zombie Aqua, Biolegend) for 30 min at ambient temperature. After washing, the cells were resuspended in PBS containing 2% SVF and 2 mM EDTA then labelled with the APC Vio770 anti-CD14 (clone REA599) antibody from Miltenyi Biotec, used at 1/100°. For the intracellular labelling of TNFα and IL-1β, the “Inside Stain” kit (Miltenyi Biotec) was used according to the manufacturer's protocol. The cells were fixed for 20 min at ambient temperature with 250 μL Inside Fix solution then labelled in 100 μL Inside Perm solution containing the PE anti-TNFα (clone cA2, Miltenyi Biotec) and/or APC anti-IL-1β (clone REA1172, Miltenyi Biotec) antibody at 1/50° for 30 min at ambient temperature. Data acquisition was carried out on the Canto II flow cytometer using the Diva software (BD Biosciences, San Jose, CA). The Kaluza software was used to analyze the data.

### Detection of the Cytokines

An assay of the cytokines in the supernatants of the cell cultures was carried out using the “LegendPlex Antivirus Human Panel” or “LegendPlex Human Inflammation Panel” kit (Biolegend, San Diego, USA) according to the manufacturer's instructions.

### Test for Toxicity of the Compounds on PBMCs

After 24 h of cell culture, the cell viability was determined using the CellTiter-Glo reagent (Promega), which evaluates the amount of ATP by luminescence using the EnSpire.

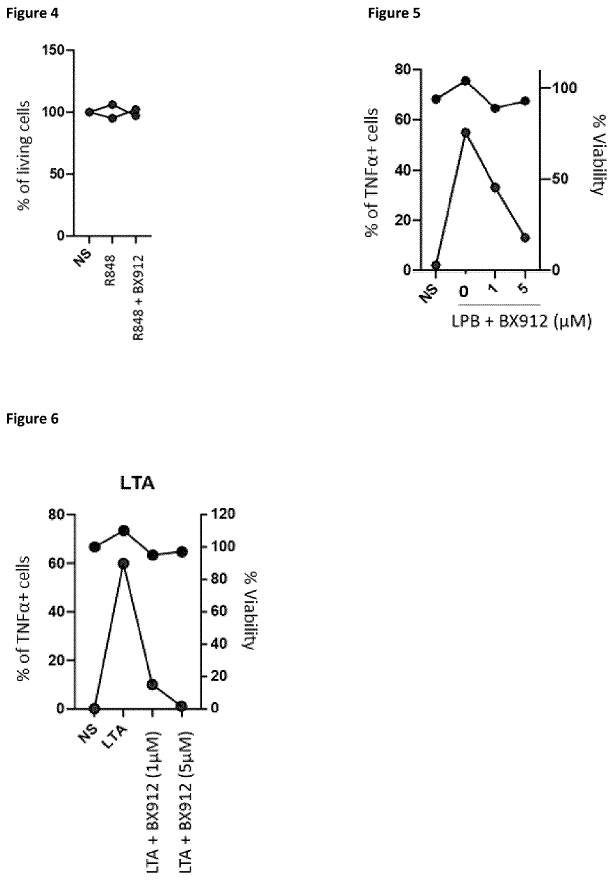

The results are presented in FIGS. 5 to 8.

### Results:

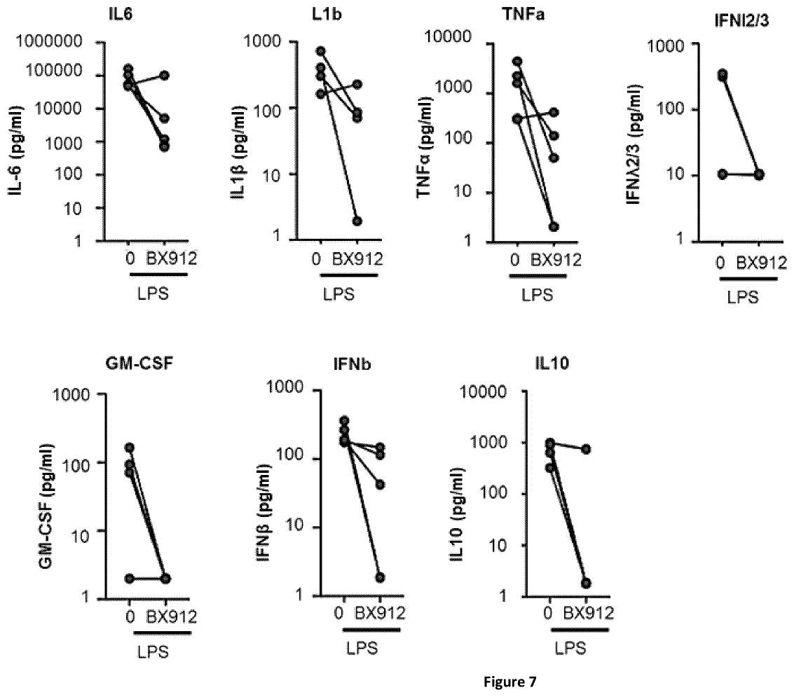

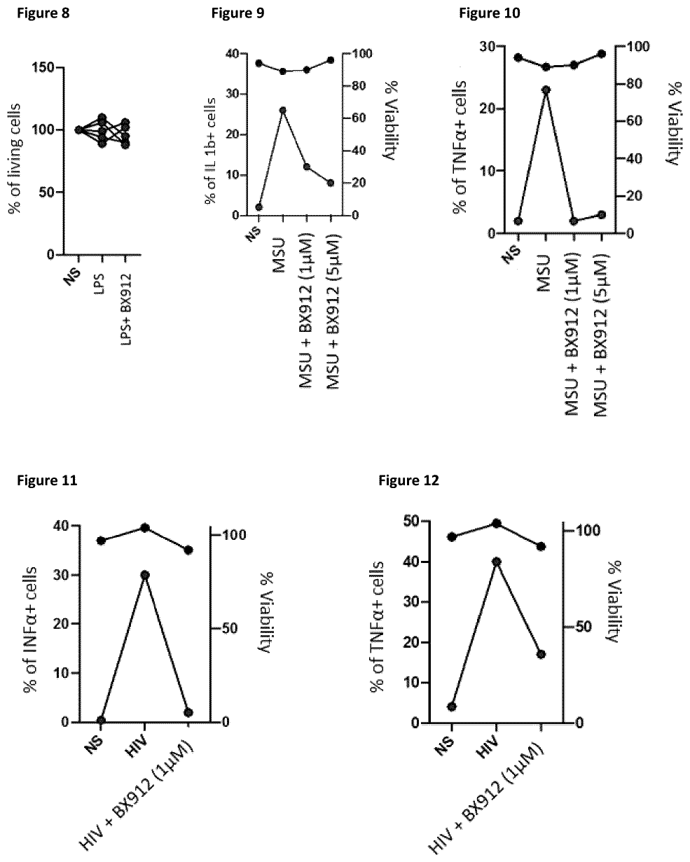

It can be seen that:
inhibition of the PDK1 protein kinase by the compound of formula (IV) (also called BX912) inhibits, according to a dosage effect, the production of inflammatory cytokines (TNFα) by the monocytes of healthy donors in response to the stimulation of the TLR4 receptors (see FIG. 5).
inhibition of the PDK1 protein kinase by the compound of formula (IV) (also called BX912) inhibits, according to a dosage effect, the production of inflammatory cytokines (TNFα) by the monocytes of healthy donors in response to the stimulation of the TLR2 receptors (see FIG. 6).
inhibition of the PDK1 protein kinase by the compound of formula (IV) (also called BX912) inhibits the secretion of the inflammatory cytokines (IL-6, IL-1β, TNFα, GM-CSF, IL-10) and the interferons (IFNI⅔, IFNβ, IFNg) by the peripheral blood mononuclear cells (PBMCs) of healthy donors in response to the stimulation of the TLR4 receptors (see FIG. 7).
the compound of formula (IV) (also called BX912) has no toxicity on the peripheral blood mononuclear cells (PBMCs) of healthy donors in response to the stimulation of the TLR4 receptors (see FIG. 8).

Thus, the compound of formula (IV) (also called BX912) has a preventive effect on the production of inflammatory cytokines (TNFα) by the monocytes and the immune cells (PBMCs) of healthy donors in response to the stimulation of the TLR 4 and TLR 2 receptors.

In addition, the compound of formula (IV) (also called BX912) is non-toxic. It has a preventive effect on the secretion of the inflammatory cytokines (IL-6, IL-1β, TNFα, GM-CSF, IL-10) and the interferons (IFN12/3, IFNβ, IFNg) by the peripheral blood mononuclear cells (PBMCs) of healthy donors in response to the stimulation of the TLR 4 receptors.

Example 4: Preventive Effect of the Inhibition of the PDK1 Protein Kinase by the Inhibitor of Formula (IV) (Also Called BX912) in Response to the Activation of the Inflammasome by Uric Acid Crystals (Mimicking “Gout”)

### Cell Stimulation

The monocytes were cultured at 1.10⁶ cells/ml. The cells were then incubated with the inhibitor of the PDK1 protein kinase of formula (IV) (also called BX912) or the inhibitor of the ROCK protein kinase of formula (VII) (also called Y27632) at the concentrations indicated for 1 h before stimulation. They were stimulated for 16 h (flow cytometry) with uric acid crystals mimicking gout (MSU) at 50 μg/ml.

The cells were recovered for the flow cytometry. For the intracellular labelling of TNFα and IL-1β, Brefeldin A (BFA) was added to the cells 30 minutes after the stimulation.

### Flow Cytometry

The cells were rinsed in PBS then incubated with a viability dye (Zombie Aqua, Biolegend) for 30 min at ambient temperature. After washing, the cells were resuspended in PBS containing 2% SVF and 2 mM EDTA then labelled with the APC Vio770 anti-CD14 (clone REA599) antibody from Miltenyi Biotec, used at 1/100°. For the intracellular labelling of TNFα and IL-1β, the “Inside Stain” kit (Miltenyi Biotec) was used according to the manufacturer's protocol. The cells were fixed for 20 min at ambient temperature with 250 μL Inside Fix solution then labelled in 100 μL Inside Perm solution containing the PE anti-TNFα (clone cA2, Miltenyi Biotec) and/or APC anti-IL-1β (clone REA1172, Miltenyi Biotec) antibody at 1/50° for 30 min at ambient temperature. Data acquisition was carried out on the Canto 11 flow cytometer using the Diva software (BD Biosciences, San Jose, CA). The Kaluza software was used to analyze the data.

The results are presented in FIGS. 9 to 10.

### Results:

It can be seen that:
inhibition of the PDK1 protein kinase by the compound of formula (IV) (also called BX912) inhibits the intracellular production of IL13 by the monocytes of healthy donors in response to an activation of the inflammasome by uric acid crystals (“gout” model) (see FIG. 9).
inhibition of the PDK1 protein kinase by the compound of formula (IV) (also called BX912) inhibits the production of TNFα by the monocytes of healthy donors in response to an activation of the inflammasome by uric acid crystals (“gout” model) (see FIG. 10).

Thus, the compound of formula (IV) (also called BX912) has a preventive effect on the intracellular production of IL-1β and TNFα on monocytes of healthy donors in response to an activation of the inflammasome by uric acid crystals (“gout” model).

### Example 5: Preventive Effect of the Inhibition of the PDK1 Protein Kinase by the Inhibitor of Formula (IV) (Also Called BX912) in Response to an Infection with the Human Immunodeficiency Virus (HIV)

### Cell Stimulation

The PBMCs were cultured at 2.10⁶ cells/ml. The plasmacytoid dendritic cells were cultured at 1.10⁶ cells/ml. The cells were then incubated with the inhibitor of the PDK1 protein kinase of formula (IV) (also called BX912) or the inhibitor of the ROCK protein kinase of formula (VII) (also called Y27632) at the concentrations indicated for 1 h before stimulation. They were stimulated for 16 h (flow cytometry) or 24 h (LegendPlex) with aldrithiol-2 (AT-2)-inactivated HIV-1MN, specific to the CXCR4 coreceptor.

The cells were recovered for the flow cytometry and the supernatants were collected for the detection of the cytokines. For the intracellular labelling of TNFα and IFNα, Brefeldin A (BFA) was added to the cells 30 minutes after the stimulation.

### Flow Cytometry

The cells were rinsed in PBS then incubated with a viability dye (Zombie Aqua, Biolegend) for 30 min at ambient temperature. After washing, the cells were resuspended in PBS containing 2% SVF and 2 mM EDTA then labelled with the APC Vio770 anti-CD14 (clone REA599) antibody from Miltenyi Biotec, used at 1/100°. For the intracellular labelling of TNFα and IFNα, the “Inside Stain” kit (Miltenyi Biotec) was used according to the manufacturer's protocol. The cells were fixed for 20 min at ambient temperature with 250 μL Inside Fix solution then labelled in 100 μL Inside Perm solution containing the APC anti-TNFα (Miltenyi Biotec) and/or PE anti-IFNα (Miltenyi Biotec) antibody at 1/50° for 30 min at ambient temperature. Data acquisition was carried out on the Canto II flow cytometer using the Diva software (BD Biosciences, San Jose, CA). The Kaluza software was used to analyze the data.

### Detection of the Cytokines

An assay of the cytokines in the supernatants of the cell cultures was carried out using the “LegendPlex Antivirus Human Panel” or “Legendplex Human Inflammation Panel” kit (Biolegend, San Diego, USA) according to the manufacturer's instructions.

### Test for Toxicity of the Compounds on PBMCs

After 24 h of cell culture, the cell viability was determined using the CellTiter-Glo reagent (Promega), which evaluates the amount of ATP by luminescence using the EnSpire.

The results are presented in FIGS. 11 to 14.

### Results:

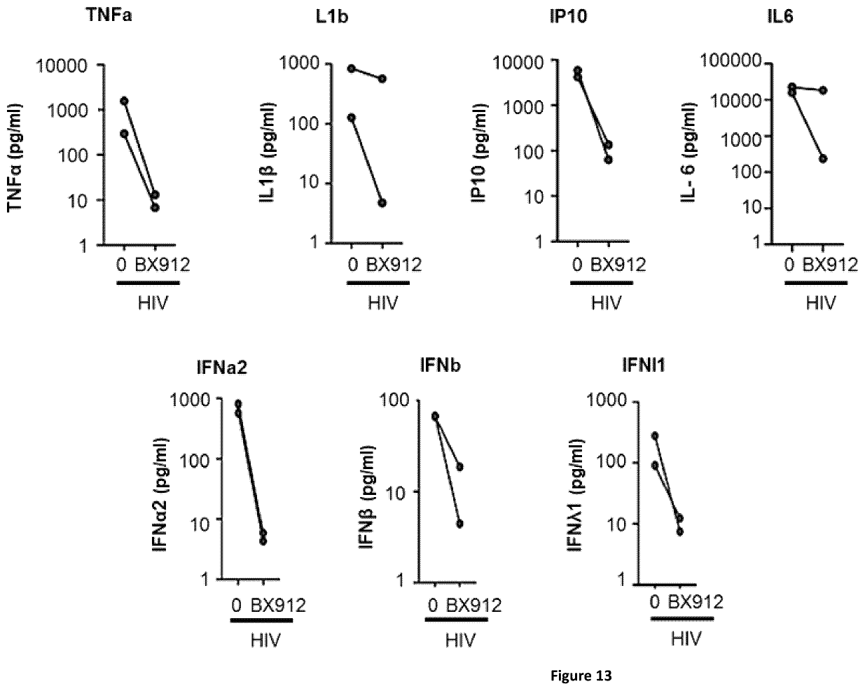

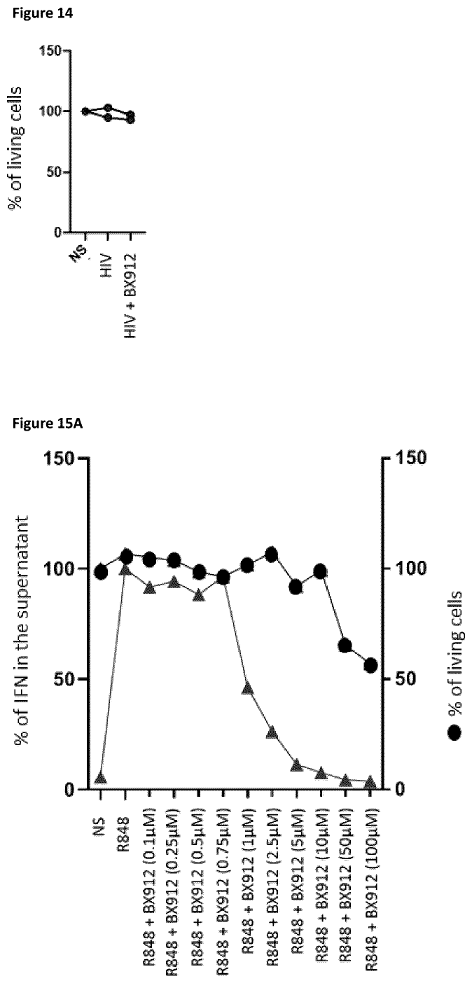

It can be seen that:
inhibition of the PDK1 protein kinase by the compound of formula (IV) (also called BX912) inhibits the intracellular production of interferon IFNα on plasmacytoid dendritic cells (pDC) of healthy donors in response to an HIV infection (see FIG. 11).
inhibition of the PDK1 protein kinase by the compound of formula (IV) (also called BX912) inhibits the production of TNFα on plasmacytoid dendritic cells (pDC) of healthy donors in response to an HIV infection (see FIG. 12).
inhibition of the PDK1 protein kinase by the compound of formula (IV) (also called BX912) inhibits the secretion of the inflammatory cytokines (IL-6, IL-1β, TNFα, P10) and the interferons (IFNα2, IFNβ, IFN11) by the peripheral blood mononuclear cells (PBMCs) of healthy donors in response to an HIV infection (see FIG. 13).
the compound of formula (IV) (also called BX912) has no toxicity on the peripheral blood mononuclear cells (PBMCs) of healthy donors infected with HIV (see FIG. 14).

Thus, the compound of formula (IV) (also called BX912) has a preventive effect on the intracellular production of interferon IFNα and TNFα on plasmacytoid dendritic cells (pDC) of healthy donors in response to an HIV infection.

In addition, the compound of formula (IV) (also called BX912) is non-toxic. It has a preventive effect on the secretion of the inflammatory cytokines (IL-6, IL-1β, TNFα, IP10) and the interferons (IFNα2, IFNβ, IFN11) by the peripheral blood mononuclear cells (PBMCs) of healthy donors in response to an HIV infection.

### Example 6: Curative Effect of the Inhibition of the PDK1 Protein Kinase by the Inhibitor of Formula (IV) (Also Called BX912) in Response to the Stimulation of the TLR7/8 Receptors

### Cell Stimulation

The PBMCs were cultured at 2.10⁶ cells/ml. The cells were then incubated with the inhibitor of the PDK1 protein kinase of formula (IV) (also called BX912) at the concentrations indicated for 1 h before stimulation, at the same time as the stimulation or 1 h after the stimulation. The PBMCs were stimulated for 24 h with the TLR7/8 agonist, Resiquimod—R848, at 5 μg/ml.

The supernatants were collected for the detection of the cytokines.

### Detection of the Cytokines

The assay of the interferons secreted by the PBMCs was carried out by means of the STING-37 reporter cell line. These are human HEK293 cells transfected with a reporter gene expressing luciferase under the control of the ISRE response element. The luciferase produced was revealed by the “Bright-Glo™ Luciferase Assay System” kit from Promega.

Test for toxicity of the compounds on the peripheral blood mononuclear cells (PBMCs).

After 24 h of cell culture, the cell viability was determined using the CellTiter-Glo reagent (Promega), which evaluates the amount of ATP by luminescence using the EnSpire.

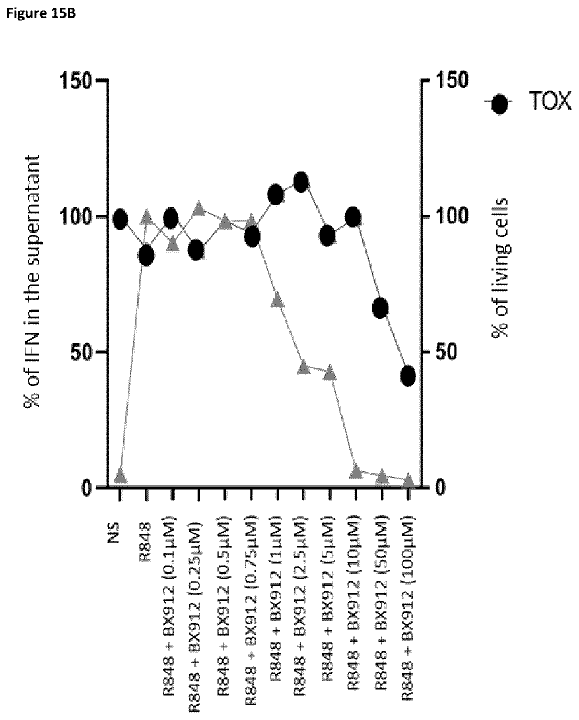

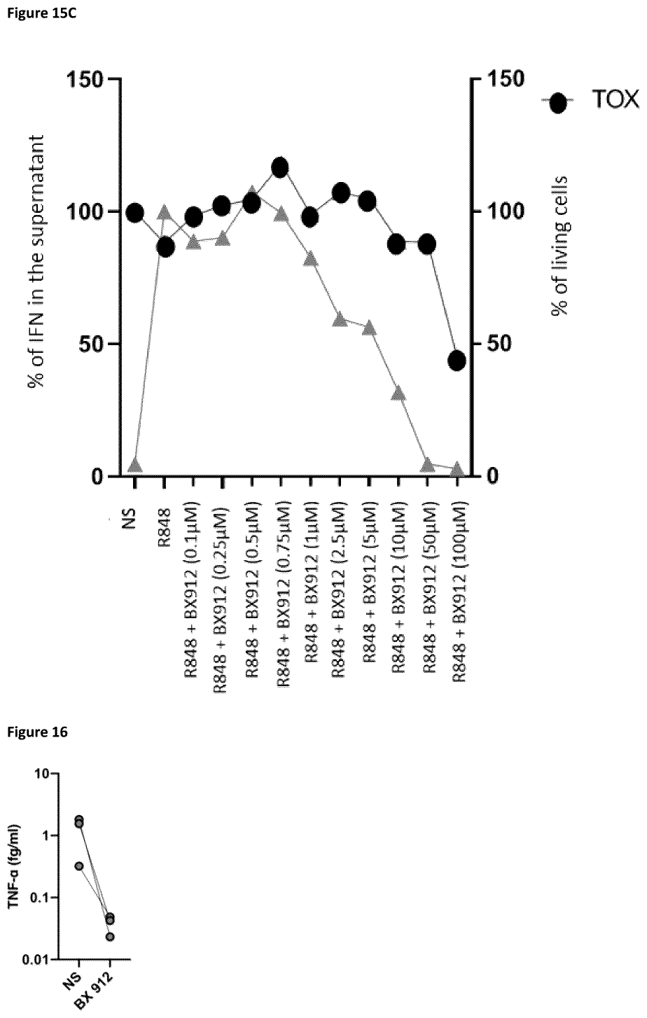

The results are presented in FIG. 15.

### Example 7: Curative Effect of the Inhibition of the PDK1 Protein Kinase by the Inhibitor of Formula (IV) (Also Called BX912) in Patients Suffering from Juvenile Idiopathic Arthritis

### Cell Culture

The PBMCs were cultured at 2.10⁶ cells/ml. The monocytes were cultured at 1.10⁶ cells/ml. The cells were then incubated with the inhibitor of the PDK1 protein kinase of formula (IV) (also called BX912) for 24 h. The supernatants were collected for the detection of the cytokines.

### Detection of the Cytokines

The assay of the TNFα secreted by the monocytes of a patient suffering from juvenile idiopathic arthritis was carried out by SIMOA (digital ELISA).

The results are presented in FIG. 16.

### Results:

It can be seen that:
inhibition of the PDK1 protein kinase by the compound of formula (IV) (also called BX912) inhibits the spontaneous production of TNFα on monocytes of patients suffering from juvenile idiopathic arthritis (FIG. 16).

Inhibition of PDK1 by BX912 makes it possible to drastically reduce the spontaneous production of inflammatory cytokines by the monocytes originating from patients suffering from juvenile idiopathic arthritis. This indicates that the compound of formula (IV) (also called BX912) can be used as a curative in patients suffering from a chronic auto-inflammation (auto-inflammatory disease, autoimmune diseases, interferonopathies).

### Example 8: Evaluation of the Efficacy of the Inhibitor of Formula (IV) (Also Called BX912) in a Murine Model of Human Rheumatoid Polyarthritis

### Experimental Protocol:

### Animals:

Seventy DBA/1J mice (Jackson Laboratories, R #4379, PO 797038, male, 7-8 weeks) were received, individually examined and housed in nine cages each with ten mice. No clinical signs of disease or distress were observed. The mice were placed in quarantine with daily inspections and tagged on the ear (SOP 810) with unique numbers for the purpose of identification.

### Preparation of a Collagen/CFA Emulsion

20 mg type II bovine collagen (Chondrex, Cat. 20021, batch 200078) was dissolved in 5 ml 0.01 M acetic acid by stirring overnight at 4-8° C. 3 ml collagen was emulsified with 3 ml complete Freund's adjuvant (CFA, Chondrex, Inc., Cat. 7001, batch 180692, 4 mg/ml Mycobacterium tuberculosis) in an ice bath in three batches. The stability of the emulsion was tested by floating a drop on water; the emulsion did not disperse.

### Experimentation:

On day 0, the mice were weighed, and injected sub-cutaneously (SC, SOP 1610) at the base of the tail with 50 μl collagen/CFA emulsion. The mice were weighed every week and scored for signs of arthritis every day. Each paw was scored and the sum of the four scores was recorded as arthritis in index (AI) The maximum possible AI was 16.

| Score | Effects |
|---|---|
| 0 | no visible effect of arthritis |
| 1 | oedema and/or erythema in one digit |
| 2 | oedema and/or erythema of 2 joints |
| 3 | oedema and/or erythema of more than 2 joints |
| 4 | severe arthritis of the whole paw and the fingers with deformation of the limbs and |
| | ankylosis of the joint. |

The mice were divided into five treatment groups, according to Table 1 below.

TABLE 1
Treatment groups

| | | Dose | | Number of |
|---|---|---|---|---|
| | | administered | | mice |
| Group | Treatment | (mg/kg) | | treated |
| 1 | Vehicle: | 10.0 | mg/kg | 10 |
| | 3 ml 1N HCl (MP Biomedicals, Cat. 194055, batch | | | |
| | 3956H) + 27 ml demineralized water. | | | |
| 2 | Prednisolone: | 10.0 | mg/kg | 10 |
| | 44.5 mg prednisolone 21-hemisuccinate sodium salt | | | |
| | (SF = 1.339, Sigma, Cat. P4153, batch BCBB6186V) was | | | |
| | dissolved in 33.2334 ml demineralized water to | | | |
| | prepare a solution of 1 mg/ml. | | | |
| 3 | BX912: | 0.5 | mg/kg | 10 |
| | 1.5 mg BX912 (Axon Medchem, Cat. 1130, batch 2) was | | | |
| | dissolved in 3 ml 1N HCl and 27 ml demineralized water | | | |
| | to prepare a solution at 0.05 mg/ml. | | | |
| 4 | BX912: | 2.0 | mg/kg | 10 |
| | 6 mg BX912 dissolved in 3 ml 1N HCl and 27 ml | | | |
| | demineralized water to prepare a solution at 0.2 | | | |
| | mg/ml. | | | |
| 5 | BX912: | 10.0 | mg/kg | 10 |

30.74 mg BX912 dissolved in 3 ml 1N HCl and 27.74 ml
demineralized water to prepare a solution at 1 mg/ml.

The mice were weighed and assessed for signs of disease. On day 42, the mice from groups 1 to 3 were anaesthetized (SOP 1810) and exsanguinated (SOP 1686) into pre-cooled EDTA tubes (Becton Dickinson, Cat. 365974, batch 9266867, exp. 28/02/21). The blood was converted into plasma (SOP 6200) which was stored in four aliquots of 55 μl in each case in labelled Eppendorf tubes at −80° C. (SOP 7020). The limbs were sampled individually in 15 ml 10% neutral buffered formalin (Richard-Allan Scientific, Cat. 5701, batch 537633, exp. 05/2024). The carcasses were disposed of in an appropriate manner (SOP 1790).

On day 45, the mice from group 4 were anaesthetized and exsanguinated into pre-cooled EDTA tubes. The blood was converted into plasma which was stored in four aliquots of 55 μd in each case in labelled Eppendorf tubes at −80° C. The limbs were sampled individually in 15 ml 10% neutral buffered formalin. The carcasses were disposed of in an appropriate manner.

On day 46, the mice from group 5 were anaesthetized and exsanguinated into pre-cooled EDTA tubes. The blood was converted into plasma which was stored in four aliquots of 55 μd in each case in labelled Eppendorf tubes at −80° C. The limbs were sampled individually in 15 ml 10% neutral buffered formalin.

The carcasses were disposed of in an appropriate manner.

Three aliquots of plasma were defrosted at ambient temperature and tested, by ELISA, for TNFα (R&D Systems, Cat. MTA001B, batch P253770), IFNγ (R&D Systems, Cat. MIF00, batch P247106) and IL-6 (R&D Systems, Cat. M600013, batch P256377, exp. 20SEP21). One aliquot of plasma was defrosted at ambient temperature and assayed, by ELISA, for IL-1su (R&D Systems, Cat. MLBC, batch P260121).

The representative limbs of each mouse, preserved in 10% neutral buffered formol, were submitted for histopathology and treated routinely. Two sections per block were collected at different depths so as to capture the appropriate sections of the largest number of possible digital and tarsal joints; the depths between the sections varied depending on the conformity and thickness of the paw. The two sections were applied to a single glass slide and stained with toluidine blue (TB). The histopathological sections were assessed by a board-certified veterinary pathologist, using optical microscopy. The histological lesions of all the joints captured were classified depending on their severity, according to the following criteria below. Each section captured different joints in the paw, so that the two sections were used to evaluate the entire sample with precision.

Evaluation of the inflammation

| Score | Effects |
|---|---|
| 0 | normal |
| 1 | minimal infiltration of inflammatory cells into the synovial membrane and/or the |
| | periarticular tissue |
| 2 | slight infiltration of inflammatory cells towards the affected joints; generally 1 to 3 |
| | affected joints |
| 3 | moderate infiltration with moderate oedema (3-4 digital joints + carpus or tarsus) |
| 4 | marked infiltration affecting most of the areas with marked oedema; 1 or 2 unaffected |
| | joints may be present |
| 5 | severe diffuse infiltration with severe oedema; all of the joints and all of the |
| | periarticular tissues affected |

Evaluation of the pannus

| Score | Effects |
|---|---|
| 0 | normal |
| 1 | minimal infiltration of the pannus along the marginal areas in the cartilage and |
| | subchondral bone |
| 2 | slight infiltration with destruction of the marginal area of the hard tissues in the affected |
| | joints |
| 3 | moderate infiltration with moderate destruction of the hard tissues in the affected |
| | joints |
| 4 | marked infiltration with marked destruction of the articular architecture, affecting most |
| | of the joints |
| 5 | severe infiltration associated with a total or almost total destruction of the articular |
| | architecture, affects all of the joints |

Evaluation of the damage to the cartilage

| Score | Effects |
|---|---|
| 0 | normal |
| 1 | minimal; minimal to slight loss of proteoglycan but no obvious loss of chondrocytes or |
| | disturbance of the collagen in the affected joints |
| 2 | mild; slight loss of proteoglycan with focal areas of loss of chondrocytes and/or of |
| | disturbance of the collagen in certain affected joints |
| 3 | moderate; moderate loss of proteoglycan with moderate multifocal loss of |
| | chondrocytes (depth to the median area) and/or disturbance of the collagen in the |
| | affected joints, a part of the matrix remains on every affected surface with areas of |
| | severe loss of matrix |
| 4 | marked; marked loss of proteoglycan with marked multifocal loss of chondrocytes |
| | (depth to deep area) and/or disturbance of the collagen in most of the joints |
| 5 | severe; severe loss of proteoglycan with severe multifocal loss of chondrocytes (depth |
| | as far as the tidemark) and/or disturbance of the collagen in all of the joints |

Evaluation of the bone resorption/damage

| Score | Effects |
|---|---|
| 0 | normal |
| 1 | minimal; small areas of resorption that are hardly visible at low magnification, rare |
| | osteoclasts in the affected joints, limited to the marginal areas |
| 2 | mild; more areas of resorption that are hardly visible at low magnification, more |
| | osteoclasts in the affected joints, limited to the marginal areas |
| 3 | moderate; obvious resorption of the medullary and cortical trabecular bone without |
| | full-thickness defects in the cortex, loss of some medullary trabeculae, lesion that is |
| | visible at low magnification, more osteoclasts in the affected joints |
| 4 | marked; full-thickness defects in the cortical bone, often with distortion of the profile |
| | of the remaining cortical surface, marked loss of the medullary bone, many osteoclasts, |
| | affects most of the joints |
| 5 | severe; full-thickness defects of the cortical bone and destruction of the articular |
| | architecture of all of the joints |

The inflammation, pannus, cartilaginous lesion and bone resorption scores for each paw were added up to obtain a total arthritis score (range from 0 to 20).

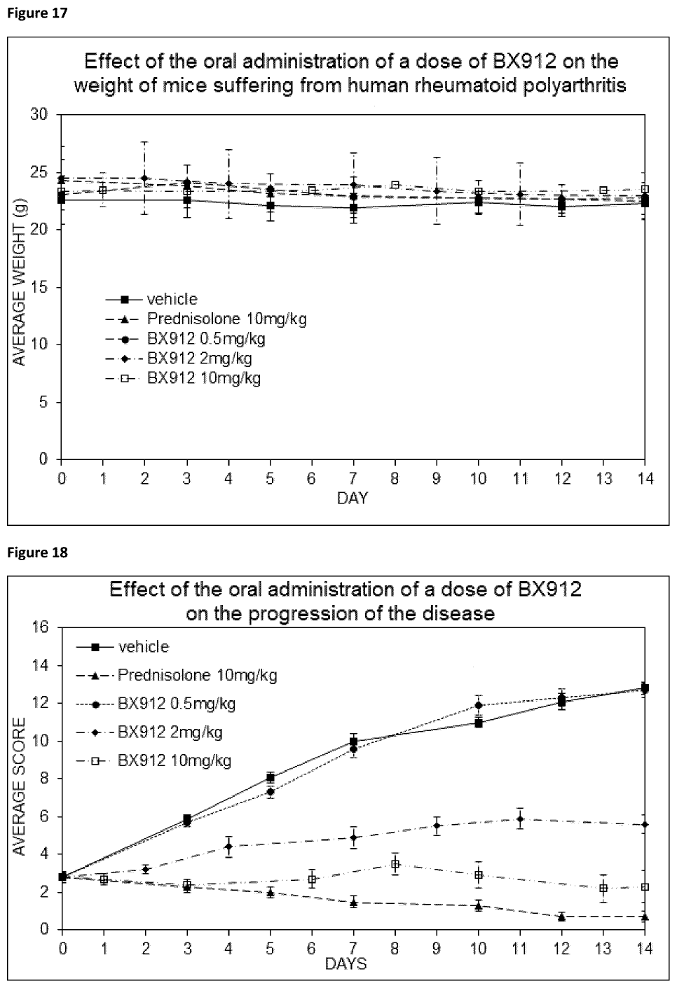

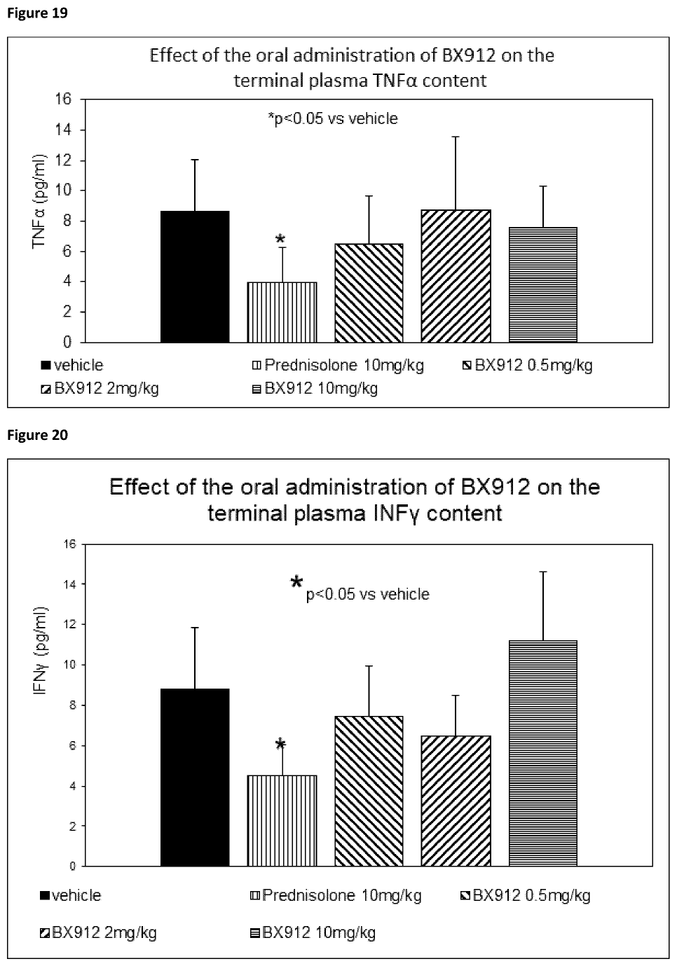

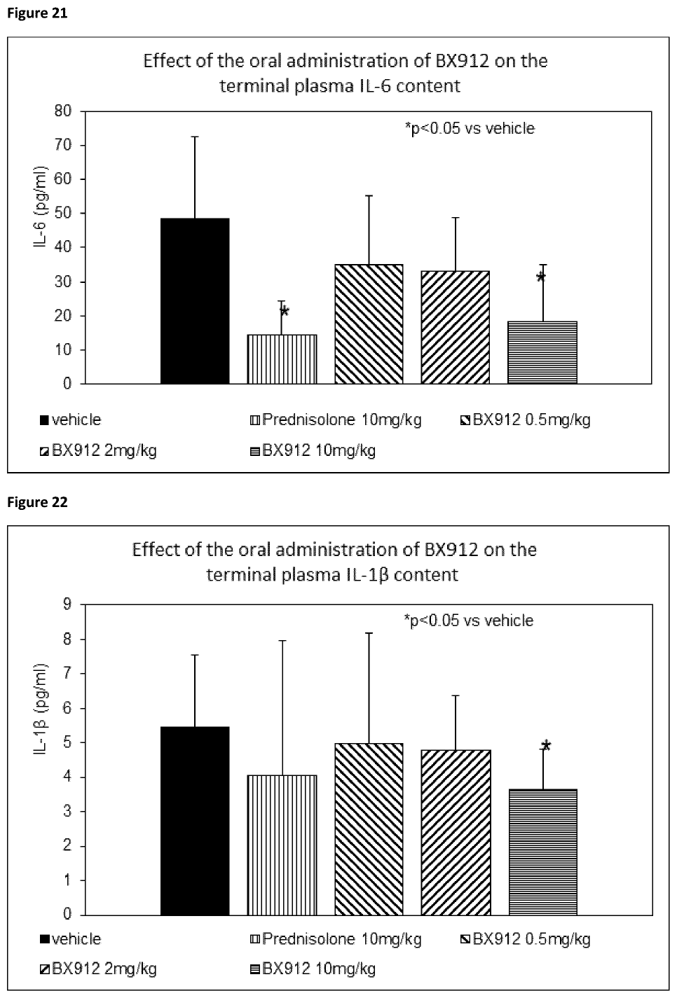

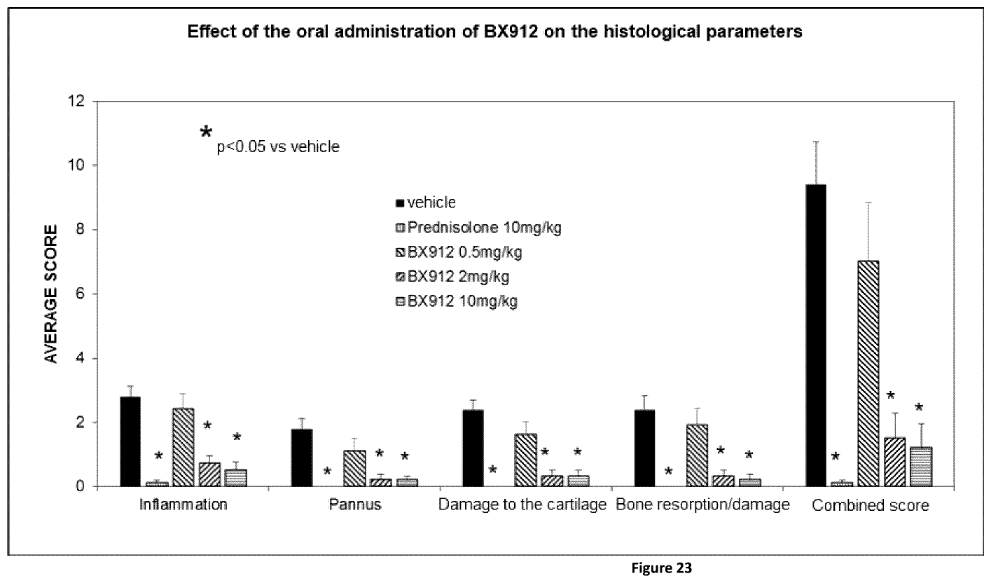

The results are presented in FIGS. 17 to 23.

### Conclusions:

### Development of the Disease:

The disease was induced by subcutaneous injection of type II bovine collagen emulsified in CFA on DAY 0 followed 21 days later by subcutaneous injection of type II bovine collagen emulsified in ICFA. The first signs of disease were recorded on DAY 27. The mice were recruited into the treatment regimen with an average group disease score (AI) of 2.8 (range—2-4). An oral administration once per day with the vehicle (0.1 N HCl, group 1) was initiated on day 28. At the end of the study on day 42, a maximum disease score of 12.8 was recorded. Terminal plasma TNFα=8.7 μg/ml, IL-1(3=5.4 μg/ml, IL-6=−49 μg/mi and IFNγ=˜9 μg/ml. Histological lesions associated with collagen-induced arthritis were present in the interphalangeal (finger) and tarsal (ankle) joints. The lesions consisted of an intra-articular and peri-articular subacute inflammation of the soft tissues, a pannus, cartilaginous lesions and a bone resorption/lesion.

Effect of the Therapeutic Treatment with Prednisolone (Group 2):

Daily oral administration of 10 mg/kg from DAY 28 had no significant effect on the weight of the mouse. This treatment pattern caused an immediate reduction in the severity of the signs of the disease, so that at the end of the study a significant reduction of 95% in the severity of the disease was observed. This therapeutic pattern caused a significant decrease in plasma TNFα (54%), IL-6 (70%) and IFNγ (49%). The total average arthritis scores were lowest in the animals treated with prednisolone.

Effect of the Therapeutic Treatment with BX912 (Groups 3-5):

A daily oral administration of BX912 caused a dose-dependent reduction in the rate of progression of the disease and in the severity of the disease. At the lowest dose (0.5 mg/kg, group 3), no ameliorative effect was observed. At the median dose (2 mg/kg, group 4), a significant reduction in the rate of progression of the disease led to a significant reduction of 56% in the overall severity of the disease at the end of the study. At the highest dose (10 mg/kg, group 5), the progression of the disease was stopped, causing a significant reduction of 82% in the overall severity of the disease at the end of the study, which was associated with a significant reduction in the terminal plasma levels of IL-1i (37%) and IL-6 (62%). The treatment with the medium (2 mg/kg; group 4) and high (10 mg/kg; group 5) doses of BX912 caused similar marked reductions in the total average arthritis scores with respect to the control vehicle (group 1); the treatment with the low dose (0.5 mg/kg; group 3) caused slight reductions with respect to the control vehicle. The reductions observed with the BX912 treatments at a medium and high dose (groups 4 and 5, respectively) did not quite reach the degree of efficacy observed with the treatment with prednisolone (group 2), but the reductions with these therapeutic patterns were still substantial. Similar overall trends were observed with the individual histopathological components of the total score.

### Example 9: Effect of the Inhibition of the PDK1 Protein Kinase by the Inhibitor of Formula (IV) (Also Called BX912) on the Immune Cells (T Lymphocytes and B Lymphocytes)

### Isolation and Culturing of Human Leukocytes.

The in vitro experiments were carried out using human peripheral blood mononuclear cells (PBMCs) isolated by density gradient centrifugation by means of a medium for separating leukocytes from peripheral blood (STEMCELL Technologies). The blood of healthy donors was obtained from the “Etablissement Frangais du Sang” [French Blood Establishment] (convention #07/CABANEL/10⁶; Paris, France). The studies on material from patients were approved by the research ethics committee (ID-RCB/EUDRACT: 2014-A01017-40 and 2018-A01358-47). The T lymphocytes were purified by negative selection, using the “Pan T Cell Isolation Kit” from Miltenyi. The B lymphocytes were purified by negative selection, using the “Pan B Cell Isolation Kit” from Miltenyi. The T lymphocytes and B lymphocytes were cultured in RPMI 1640 (Sigma) containing 10% inactivated foetal bovine serum and 1 mM glutamine (Hyclone, Logan, UT).

### Cell Stimulation

The T lymphocytes and B lymphocytes were cultured at 1.10⁶ cells/ml. The cells were then incubated with the inhibitor of the PDK1 protein kinase of formula (IV) (also called BX912) at the concentrations indicated for 1 h before stimulation. The T lymphocytes were stimulated for 3 days with beads from the “Dynabeads Human T-Activator CD3/CD28” kit from Thermo Fisher Scientific, following their instructions. The cells were recovered for the flow cytometry. The B lymphocytes were stimulated for 24 h with CpG-A at 1 μg/ml. The cells were recovered for the flow cytometry.

### Flow Cytometry

The cells were rinsed in PBS then incubated with a viability dye (Zombie Aqua, Biolegend) for 30 min at ambient temperature. After washing, the cells were resuspended in PBS containing 2% SVF and 2 mM EDTA. For the T lymphocytes, the cells were suspended in PBS containing 2% SVF and 2 mM EDTA then labelled with the PE anti-CD69 and APC anti-CD3 antibody from Miltenyi Biotec, used at 1/100°. After incubation for 30 min, the cells were rinsed in PBS 2% SVF and 2 mM EDTA. Data acquisition was carried out on the Attune N×T flow cytometer from Thermo Fisher. The FlowJo software was used to analyze the data. For the B lymphocytes, the cells were resuspended in PBS containing 2% SVF and 2 mM EDTA then labelled with the PE anti-CD69 and PE.Vio770 anti-CD19 antibody from Miltenyi Biotec, used at 1/100°. After incubation for 30 min, the cells were rinsed in PBS containing 2% SVF and 2 mM EDTA. Data acquisition was carried out on the Attune N×T flow cytometer from Thermo Fisher. The FlowJo software was used to analyze the data.

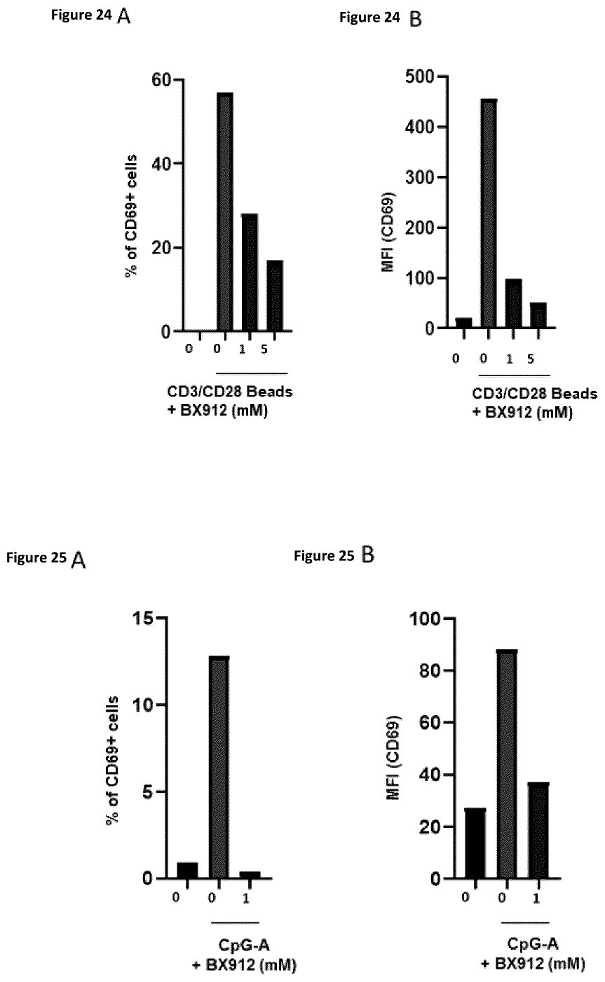

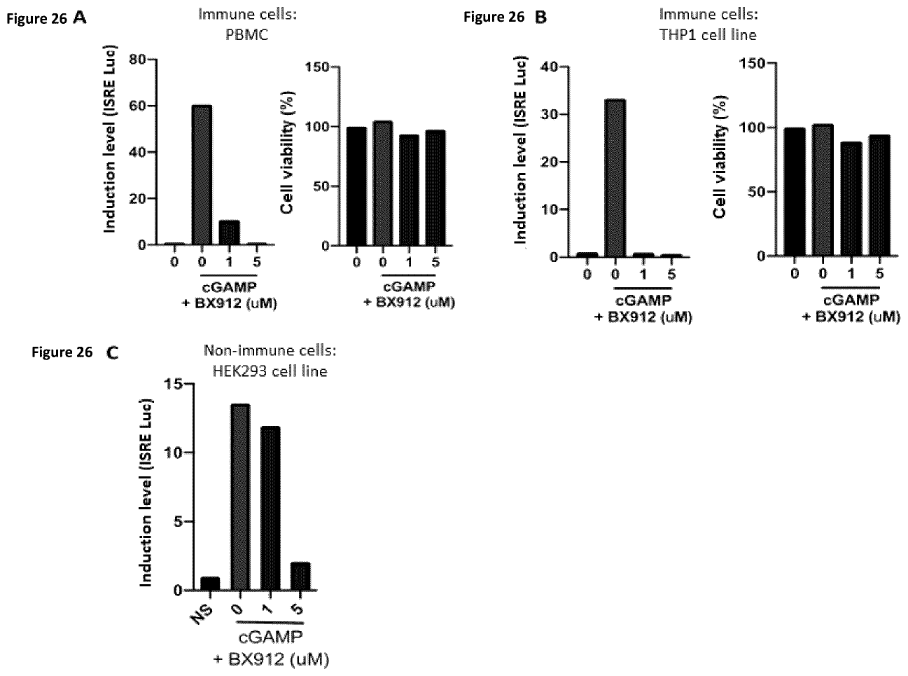

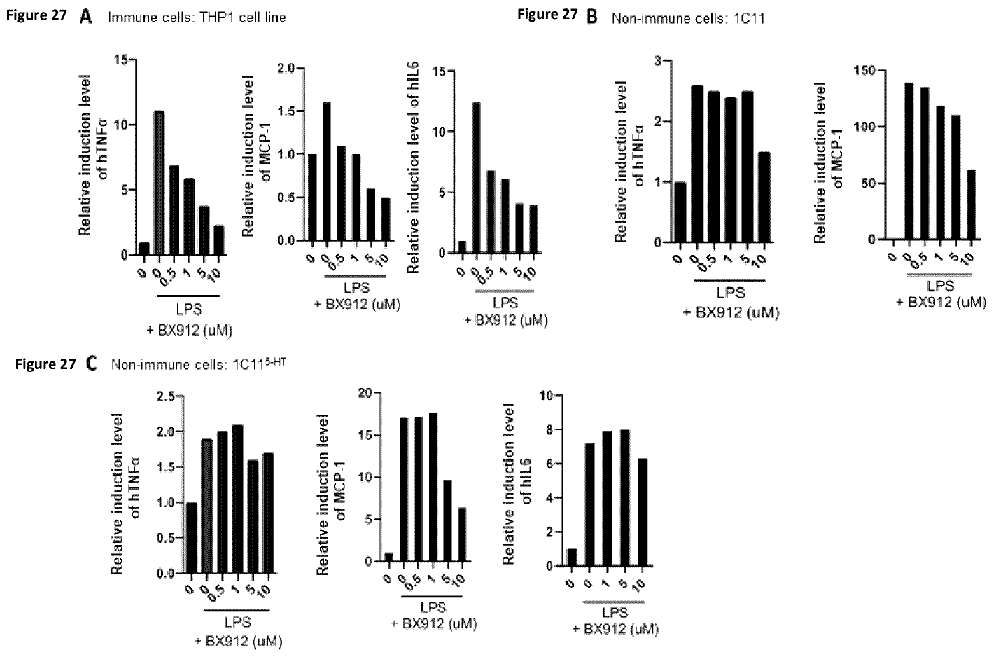

The results are presented in FIGS. 24 and 25.

### Results:

It can be seen that:
inhibition of the PDK1 protein kinase by the compound of formula (IV) (also called BX912) inhibits, according to a dosage effect, the expression of the CD69 marker induced by the CD3/CD28 beads, according to a dosage effect, at the surface of the T lymphocytes, without affecting the viability thereof (FIG. 24).
inhibition of the PDK1 protein kinase by the compound of formula (IV) (also called BX912) inhibits, according to a dosage effect, the expression of the CD69 marker induced by CpG-A, ligand of TLR9, according to a dosage effect, at the surface of the B lymphocytes, without affecting the viability thereof (FIG. 25).

Thus, the compound of formula (IV) (also called BX912) has a preventive effect on the activation of the T and B lymphocytes. The compound of formula (IV) (also called BX912) has an anti-inflammatory and anti-interferon effect on the T and B lymphocytes. The compound of formula (IV) (also called BX912) has an immunosuppressive effect on the T and B lymphocytes.

### Example 10: Specific Anti-Inflammatory Effect of the Inhibitor of the PDK1 Protein Kinase of Formula (IV) (Also Called BX912) on the Immune Cells in Comparison with the Non-Immune Cells

Isolation and culturing of human leukocytes.

The in vitro experiments were carried out using human peripheral blood mononuclear cells (PBMCs) isolated by density gradient centrifugation by means of a medium for separating leukocytes from peripheral blood (STEMCELL Technologies). The blood of healthy donors was obtained from the “Etablissement Frangais du Sang” [French Blood Establishment] (convention #07/CABANEL/10⁶; Paris, France). The peripheral blood mononuclear cells (PBMCs) and the THP1-Dual monocytes were cultured in RPMI 1640 (Sigma) containing 10% inactivated foetal bovine serum and 1 mM glutamine (Hyclone, Logan, UT).

Culturing the 1C11 Neuronal Stem Cells and Differentiation into 1C11^{5-HT} Serotonergic Neurons.

The 1C11 neuroectodermal stem cells were cultured in DMEM-Glutamax (Life Sciences) containing 10% foetal bovine serum (Biochrom). The 1C11 cells were made to differentiate into 1C11^{5-HT} serotonergic neurons by addition of dibutyryl-cyclic AMP (dbcAMP, 1 mM, Sigma). The complete serotonergic neuronal phenotype is obtained after 4 days of differentiation.

### Culturing the Human Embryonic Kidney Cells (HEK293 Cell Line)

The HEK293 were cultured in DMEM (Sigma) containing 10% inactivated foetal bovine serum and 1% penicillin and streptomycin.

### Cell Stimulation

The PBMCs were cultured at 2.10⁶ cells/ml. The THP1-Dual, HEK293, 1C11 and 1C11^{5-HT} cell lines were cultured at 1.10⁶ cells/ml. The cells were then incubated with the inhibitor of the PDK1 protein kinase of formula (IV) (also called BX912) at the concentrations indicated for 1 h before stimulation. The PBMCs and HEK293 were stimulated for 24 h with the STING agonist, cGAMP, at 3 μg/ml. The culture supernatants of the PBMCs were collected for the detection of the cytokines. The THP1-Dual (InvivoGen) were stimulated for 24 h with the STING agonist at 3 μg/ml or the TLR4 agonist at 100 ng/ml. The interferon response of the THP1-Dual possessed by the ISRE-ISG-Luciferase construction was revealed by means of the Quanti-Luc detection agent, following the supplier's instructions. The 1C11 stem cells and the 1C115-HT serotonergic neurons were activated for 1 h with the TLR4 agonist: LPS at 100 ng/ml⁻¹. The cells were washed in PBS and lysed to prepare the mRNA with a view to an analysis by RT-qPCR.

### Detection of the Cytokines

The assay of the interferons secreted by the PBMCs was carried out by means of the STING-37 reporter cell line. These are human HEK293 cells transfected with a reporter gene expressing luciferase under the control of the ISRE response element. The culture supernatants of the PBMCs were added to STING-37 cultures at 0.4×10⁶ cells/ml for 24 h. The luciferase produced was revealed by the “Bright-Glo™ Luciferase Assay System” kit from Promega.

The experiments on the HEK293 non-immune cells were carried out on STING-37 (HEK293 containing the ISRE-Luciferase construction). The production of interferons was revealed with the “Bright-Glo™ Luciferase Assay System” kit from Promega.

### Toxicity Test

The cell toxicity of the compounds was measured by quantifying ATP with CellTiter Glo (Promega).

Detection of the mRNA Encoding TNFα, IL6 and MCP1 by RT-qPCR

The total RNA is extracted from the cells with the E.Z.N.A. Total RNA Kit 1 kit (Omega Bio-tek) according to the supplier's instructions. The RNA concentration is measured using a spectrophotometer (Nanodrop ND-1000). The total RNA is reverse-transcribed to cDNA using the Prime Script RT Master Mix kit (Takara RR036A) according to the supplier's instructions.

The real-time quantitative PCR is performed according to the following conditions: cDNA diluted to 1/10^{th} is added to the following mixture: 3.8 μL H₂O, 0.1 μl sense and antisense primers (10 μM) (Table 1), 5 μl of the mixture Absolute qPCR SYBR Green containing Thermostart DNA polymerase, MgCl₂, SYBR Green and dNTPs (Eurogentec). The assays are carried out in triplicate in a 384-well plate (Thermo Scientific). Amplification of the DNA by quantitative PCR is carried out in the CFX384 appliance (Bio-Rad) with the following programme: 3 min at 95° C./5 sec at 95° C. and 30 sec at 60° C. repeated 40 cycles/2 min at 72° C. and 30 sec at 95° C. followed by a dissociation step which makes it possible to verify the specificity of the amplification product. The DNA amplification data are analyzed with the Bio-Rad CFX Manager software.

TABLE 2
Sequences of the primers used

| Name | Sense primer | Antisense primer |
|---|---|---|
| Mus_S16 | AGGAGCGATTTGCTGGTGTGG | CTACCAGGGCCTTTGAGATG |
| | (SEQ ID No. 1) | (SEQ ID No. 2) |
| Homo_RPL13 | AACAGCTCATGAGGCTACGG | TGGGTCTTGAGGACCTCTGT |
| | (SEQ ID No. 3) | (SEQ ID No. 4) |
| Mus_TNF | CTACTCCCAGGTTCTCTTCAA | GCAGAGAGGAGGTTGACTTTC |
| | (SEQ ID No. 5) | (SEQ ID No. 6) |
| Homo_TNF | CCTGCTGCACTTTGGAGTGA | GAGGGTTTGCTACAACATGGG |
| | (SEQ ID No. 7) | (SEQ ID No. 8) |
| Mus_MCP1 | TAGGCTGGAGAGCTACAAGAGG | AGTGCTTGAGGTGGTTGTGG |
| | (SEQ ID No. 9) | (SEQ ID No. 10) |
| Homo_MCP1 | AGAATCACCAGCAGCAAGTGTCC | ATGGAATCCTGAACCCACTTCTGC |
| | (SEQ ID No. 11) | (SEQ ID No. 12) |
| Mus_IL6 | GATGGATGCTACCAAACTGGA | GGAAATTGGGGTAGGAAGGA |
| | (SEQ ID No. 13) | (SEQ ID No. 14) |
| Homo_IL6 | GACAGCCACTCACCTCTTCA | CCTCTTTGCTGCTTTCACAC |
| | (SEQ ID No. 15) | (SEQ ID No. 16) |

### Results:

The anti-inflammatory activity of the inhibitor of the PDK1 protein kinase of formula (IV) (compound BX912) was tested on different cell types: peripheral blood mononuclear cells (PBMCs) isolated from the blood of healthy individuals (FIG. 24A), the THP1 human monocytic cell line (FIG. 241B) and the HEK293 non-immune cell line, a human embryonic kidney cell line (FIG. 24C). These cells were stimulated by cyclic guanosine monophosphate-adenosine monophosphate (cGAMP), an agent mimicking a viral infection by binding on the STING antiviral sensor. Activation of this pathway induces the production of type I interferon. The inhibitor of the PDK1 protein kinase of formula (IV) (compound BX912) blocks the production of interferons (IFN) induced by the cGAMP according to a dosage effect starting from 1 μM without affecting the viability of the PBMCs (FIG. 24A) and the THP1-Dual (FIG. 241B). On the HEK293 non-immune cells, the inhibitor of PDK1 does not block the production of interferon, at 1 μM, induced by the STING ligand (FIG. 24C). These results demonstrate that the inhibitor of the PDK1 protein kinase of formula (IV) (compound BX912) exerts an immunosuppressive effect which, in a dose-dependent manner, is specific to the immune cells.

The anti-inflammatory activity of the inhibitor of the PDK1 protein kinase of formula (IV) (compound BX912) was tested on different cell types which were activated with LPS, ligand of the Toll-like type 4 receptors (TLR4), an agent which mimics a bacterial infection. The THP1-Dual human monocytic cell line (FIG. 25A) as well as a non-immune cell line, the 1C11 neuroectodermal cell line. The 1C11 neuroectodermal stem cells (FIG. 25B) and the 1C11^{5-HT} serotonergic neurons resulting from the differentiation of the 1C11 stem cells (FIG. 25C) were activated with the ligand of TLR4. Activation of this pathway induces the production of pro-inflammatory cytokines including TNFα, IL6 and MCP1 as a result of the transcriptional stimulation of the gene encoding TNFα, IL6 and MCP1. The inhibitor of the PDK1 protein kinase of formula (IV) (compound BX912) reduces the transcription of the gene encoding TNFα, IL6 and MCP1 induced by LPS, according to a dosage effect, starting from 0.5 μM in the THP1-Dual monocytes (FIG. 25A). However, the inhibitor of the PDK1 protein kinase of formula (IV) (compound BX912) does not block the transcription of the genes encoding TNFα, IL6 and MCP1 induced by LPS in the non-immune cells, i.e. the 1C11 stem cells (FIG. 25B) and the 1C11^{5-HT}serotonergic neurons (FIG. 25C). These results confirm that the inhibitor of the PDK1 protein kinase of formula (IV) (compound BX912) exerts an immunosuppressive effect which, in a dose-dependent manner, is specific to the immune cells.

1. An inhibitor of the ROCK-PDK1 protein kinase complex for use as an anti-inflammatory and anti-interferon agent, characterized in that said inhibitor of the ROCK-PDK1 protein kinase complex is an inhibitor of the PDK1 protein kinase, and in that the inhibitor of the PDK1 protein kinase inhibits the secretion of inflammatory cytokines and of the interferons by an immune cell, when it is placed in contact with said immune cell.

2. The inhibitor of the ROCK-PDK1 protein kinase complex according to claim 1, in which the inhibitor of the ROCK-PDK1 protein kinase complex is selected from the compounds of formula (I) to (VI): 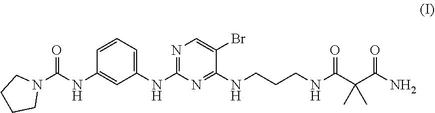 [SMILES: CC(C)(C(N)=O)C(=O)NCCCNc1nc(Nc2cccc(NC(=O)N3CCCC3)c2)ncc1Br]
or one of the pharmaceutically acceptable salts thereof, 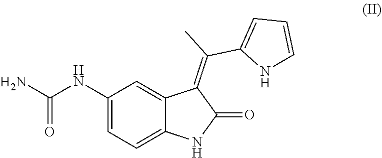 [SMILES: C/C(=C1/C(=O)Nc2ccc(NC(N)=O)cc21)c1ccc[nH]1]
or one of the pharmaceutically acceptable salts thereof, 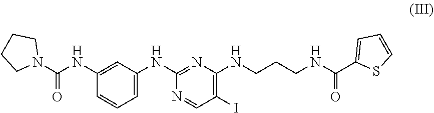 [SMILES: O=C(NCCCNc1nc(Nc2cccc(NC(=O)N3CCCC3)c2)ncc1I)c1cccs1]
or one of the pharmaceutically acceptable salts thereof, 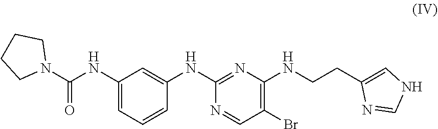 [SMILES: O=C(Nc1cccc(Nc2ncc(Br)c(NCCc3c[nH]cn3)n2)c1)N1CCCC1]
or one of the pharmaceutically acceptable salts thereof, 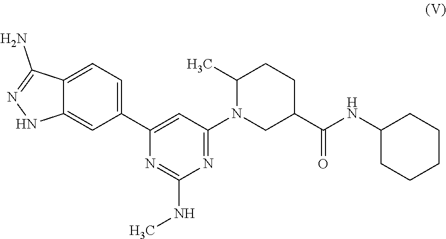 [SMILES: CNc1nc(-c2ccc3c(N)n[nH]c3c2)cc(N2CC(C(=O)NC3CCCCC3)CCC2C)n1]
or one of the pharmaceutically acceptable salts thereof, 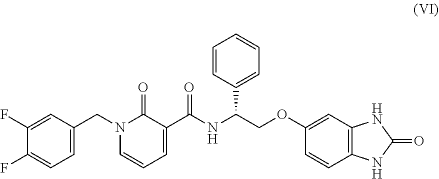 [SMILES: O=C(N[C@@H](COc1ccc2[nH]c(=O)[nH]c2c1)c1ccccc1)c1cccn(Cc2ccc(F)c(F)c2)c1=O]
or one of the pharmaceutically acceptable salts thereof.

3. The inhibitor of the ROCK-PDK1 protein kinase complex according to claim 1, in which the inhibitor of the ROCK-PDK1 protein kinase complex is the compound: 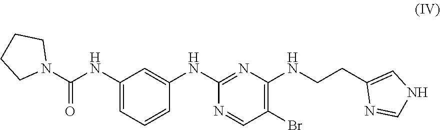 [SMILES: O=C(Nc1cccc(Nc2ncc(Br)c(NCCc3c[nH]cn3)n2)c1)N1CCCC1]
or a pharmaceutically acceptable salt thereof.

4. A pharmaceutical composition comprising at least one inhibitor of the ROCK-PDK1 protein kinase complex according to claim 1 as active principle and at least one pharmaceutically acceptable excipient and/or carrier and/or a diluent and/or a pharmaceutically acceptable vehicle.

5. The pharmaceutical composition according to claim 4, in which the inhibitor of the ROCK-PDK1 protein kinase complex is selected from the compounds of formula (I) to (VI): 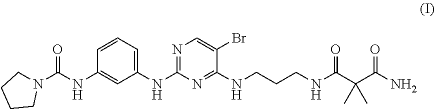 [SMILES: CC(C)(C(N)=O)C(=O)NCCCNc1nc(Nc2cccc(NC(=O)N3CCCC3)c2)ncc1Br]
or one of the pharmaceutically acceptable salts thereof, 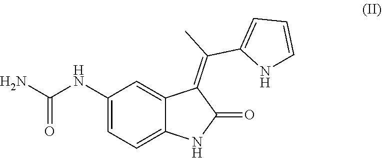 [SMILES: C/C(=C1/C(=O)Nc2ccc(NC(N)=O)cc21)c1ccc[nH]1]
or one of the pharmaceutically acceptable salts thereof, 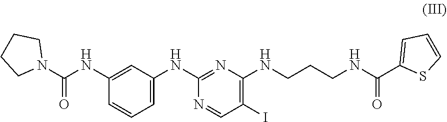 [SMILES: O=C(NCCCNc1nc(Nc2cccc(NC(=O)N3CCCC3)c2)ncc1I)c1cccs1]
or one of the pharmaceutically acceptable salts thereof, 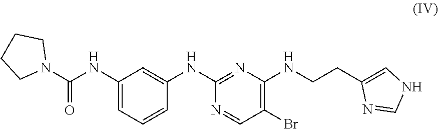 [SMILES: O=C(Nc1cccc(Nc2ncc(Br)c(NCCc3c[nH]cn3)n2)c1)N1CCCC1]
or one of the pharmaceutically acceptable salts thereof, 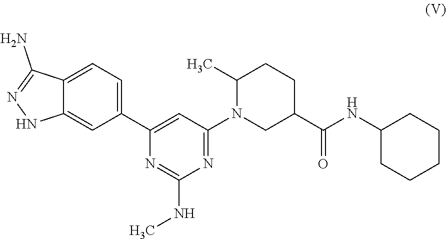 [SMILES: CNc1nc(-c2ccc3c(N)n[nH]c3c2)cc(N2CC(C(=O)NC3CCCCC3)CCC2C)n1]
or one of the pharmaceutically acceptable salts thereof 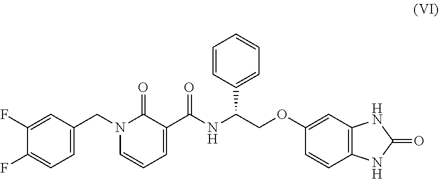 [SMILES: O=C(N[C@@H](COc1ccc2[nH]c(=O)[nH]c2c1)c1ccccc1)c1cccn(Cc2ccc(F)c(F)c2)c1=O]
or one of the pharmaceutically acceptable salts thereof.

6. A method comprising administering to a patient the pharmaceutical composition according to claim 4, in which the inhibitor of the ROCK-PDK1 protein kinase complex as active principle is administered to the patient at a dose of 0.5 mg to 30 mg per kg of bodyweight.

7. A method comprising administering to a patient the pharmaceutical composition according to claim 4, in which the inhibitor of the ROCK-PDK1 protein kinase complex as active principle is administered to the patient at a rate of 100 mg to 500 mg per day.

8. A method comprising administering to a patient the pharmaceutical composition according to claim 4, characterized in that the pharmaceutical composition is administered at the rate of one, two, three, four, five, six or seven times per week.

9. The pharmaceutical composition according to, characterized in that the pharmaceutical composition also comprises at least one second active principle.

10. The pharmaceutical composition according to claim 9, characterized in that the pharmaceutical composition is adapted for a simultaneous administration or a sequential administration of the inhibitor of the ROCK-PDK1 protein kinase complex and of the second active principle.

11. The pharmaceutical composition according to claim 4, in which the pharmaceutical composition is in a form adapted for administration thereof by the oral, parenteral or topical route.

12. A method comprising administering the pharmaceutical composition of claim 4 to a patient with an inflammatory disease.

13. The method of claim 12, in which inflammatory disease is selected from: the amyloid neurodegenerative diseases, Alzheimer's disease, prions, Parkinson's disease, amyotrophic lateral sclerosis; inflammatory diseases of intestine, chronic inflammatory bowel disease (CIBD), irritable bowel syndrome (IBS), Crohn's disease, ulcerative colitis, hemorrhagic rectal ulcer, lesions associated with Behget's disease, pouchitis, ulcerative colitis, ileitis and enteritis; acute inflammatory diseases, gout, septic shock, chronic inflammatory lumbar pain, inflammatory diseases of skin or dermatitis, psoriasis, atopic dermatitis, rosacea, acne erythematosa, acne, common warts, bullous skin diseases, contact eczema, skin cancers, rubor, erythemas, telangiectasias, inflammations of skin linked to exposure to UV, photo-irritation, photo-sensitization, photo-ageing, photo-carcinogenesis, lymphatic venous insufficiencies or heavy leg syndrome; arthritis, osteoarthritis, rheumatoid arthritis, juvenile idiopathic arthritis and psoriatic arthritis; chronic obstructive pulmonary disease (COPD); coeliac disease; chronic pancreatitis; Hashimoto's thyroiditis; primary biliary cirrhosis; sclerosing cholangitis; autoimmune hepatitis; vasculitides, systemic vasculitides associated with ANCA (anti-neutrophil cytoplasmic antibodies); spondyloarthropathies; chronic atrophying polychondritis; diabetes; scleroderma and cancer.

14. A method comprising administering the pharmaceutical composition of claim 4 to a patient with a viral and/or bacterial infection.

15. The method of claim 14, in which the viral infection is selected from: infections due to influenza virus, human immunodeficiency virus (HIV), the herpes virus or herpes simplex virus (HSV), coronavirus, COVID-19, encephalomyocarditis virus, arboviruses, chikungunya, o'nyong'nyong, Ross River, Sindbis, Mayaro viruses, yellow fever virus, dengue fever virus, Japanese encephalitis virus, West Nile virus, temperate Eurasian tick-borne encephalitis viruses, Kyasanur Forest disease viruses, Omsk hemorrhagic fever virus, Bunyavirales viruses, Bunyamwera virus, Rift Valley fever virus, Crimean-Congo hemorrhagic fever virus, hepatitis A, B and C virus and influenza virus.

16. The method of claim 14, in which the bacterial infection is selected from: infections due to gram-positive bacteria, bacteria of genus Staphylococcus, Staphylococcus aureus, and bacteria of genus Enterococcus, Enterococcus faecalis; infections due to gram-negative bacteria, bacteria of genus Escherichia, Escherichia coli, bacteria of genus Pseudomonas, Pseudomonas aeruginosa, and bacteria of genus Acinetobacter, Acinetobacter baumannii.

17. A method comprising administering the pharmaceutical composition of claim 4 to a patient with an autoimmune disease.

18. The method of claim 17, in which the autoimmune disease is selected from: disseminated lupus erythematosus, systemic lupus erythematosus, multiple sclerosis, rheumatoid polyarthritis, insulin-dependent diabetes, thrombotic thrombocytopenic purpura (TTP), graft or organ rejection, graft versus host disease, different types of sclerosis, primary Sjögren's syndrome (or Gougerot-Sjögren syndrome), autoimmune polyneuropathies, multiple sclerosis, type I diabetes, autoimmune hepatitises, ankylosing spondylitis, Reiter's syndrome, gout arthritis, chronic Hashimoto's thyroiditis (hypothyroidism), Addison's disease, autoimmune hepatitises, Basedow's disease (hyperthyroidism), autoimmune cytopenias and other hematological complications of adult and child, acute or chronic autoimmune thrombocytopenias, autoimmune hemolytic anaemias, hemolytic disease of newborn (HDN), cold agglutinin disease, thrombotic thrombocytopenic purpura and autoimmune acquired hemophilia; Goodpasture syndrome, extra-membranous nephropathies, autoimmune bullous skin disorders, refractory myasthenia gravis, mixed cryoglobulinemias, inflammatory myositis, dermatomyositis and childhood systemic autoimmune disorders including antiphospholipid syndrome, conjunctive tissue disease, different types of sclerosis, autoimmune pulmonary inflammation, Guillain-Barre syndrome, chronic inflammatory demyelinating polyradiculoneuropathy (CIDP), autoimmune thyroiditis, diabetes mellitus, myasthenia gravis, autoimmune inflammatory disease of the eye, neuromyelitis optica (Devic's disease), sclerodermas, pemphigus, insulin-resistant diabetes, polymyositis, Biermer's anaemia, glomerulonephritis, Wegener's disease, Horton's disease, periarthritis nodosa and Churg Strauss syndrome, Still's disease, atrophic polychondritis, Behget's disease, monoclonal gammopathy, Wegener's granulomatosis, lupus, psoriatic rheumatism, sarcoidosis, collagenous colitis, dermatitis herpetiformis, familial Mediterranean fever, IgA deposition glomerulonephritis, Lambert-Eaton myasthenic syndrome, ophthalmia sympathica, Fiessinger-Leroy-Reiter syndrome and uveomeningoencephalitic syndrome.
